(19) United States
(12) Patent Application Publication
(10) Pub. No.: US 2019/0374526 A1
(43) Pub. Date: Dec. 12, 2019

(54) Substituted Heterocyclic Compounds as Inhibitors of PRDM9

(71) Applicant: Merck Sharp & Dohme Corp., Rahway, NJ (US)

(72) Inventors: Christian Fischer, Natick, MA (US); Solomon D. Kattar, Wakefield, MA (US); John M. Sanders, Hatfield, PA (US)

(73) Assignee: Merck Sharp & Dohme Corp., Rahway, NJ (US)

(21) Appl. No.: 16/433,859

(22) Filed: Jun. 6, 2019

Related U.S. Application Data
(60) Provisional application No. 62/681,321, filed on Jun. 6, 2018.

(51) Int. Cl. A61K 31/4439; A61P 35/00; C07D 413/14; C07D 471/10; A61K 31/499; A61K 31/438; A61K 31/4427; C07D 401/04; A61K 31/4545; C07D 471/08; A61K 31/439; C07D 471/04; A61K 31/437; C07D 413/12

(52) U.S. Cl. CPC A61K 31/4439; A61P 35/00; C07D 413/14; C07D 471/10; A61K 31/499; A61K 31/438; C07D 413/12; C07D 401/04; A61K 31/4545; C07D 471/08; A61K 31/439; C07D 471/04; A61K 31/437; A61K 31/4427

(57) ABSTRACT

The present invention relates to Substituted Heterocyclic Compounds of Formula (I):

R¹—R²—R³—R⁴ (I)

and pharmaceutically acceptable salts or prodrug thereof, wherein R¹, R², R³, and R⁴ are as defined herein. The present invention also relates to compositions comprising at least one Substituted Heterocyclic Compound, and methods of using the Substituted Heterocyclic Compounds for inhibiting PRDM9 activity in a subject or for treating or preventing a disease mediated by PRDM9 activity.

### FIELD OF THE INVENTION

The present invention relates to Substituted Heterocyclic Compounds, compositions comprising at least one Substituted Heterocyclic Compound, and methods of using the Substituted Heterocyclic Compounds for inhibiting PRDM9 activity and for treating or preventing a disease mediated by PRDM9 activity.

### BACKGROUND OF THE INVENTION

PR domain-containing protein 9 (PRDM9) is a PR domain-containing protein with multiple zinc fingers that catalyze trimethylation of H3K4. This methyl mark is also associated with the initiation of the short highly recombinogenic segments of DNA called recombination hot spots in yeast and mice where DNA double strand breaks occur prior to crossover events. The H3K4me3 mark is often associated with transcriptional activation and DNA repair. However, H3K4me3 marks associated with hot spots are distinct from those at transcription start sites. It was reported that 87% of double strand break hot spots in mice overlap with testis-specific H3K4me3 marks.

PRDM9 is a meiosis-specific protein that has been reported to control targeting of recombination hot spots by the SPO11/TopoVIB DNA isomerase like (TOPOVIBL) complex which generates meiosis specific DNA double strand breaks. Specifically, PRDM9 binds in vitro to a 13-mer DNA sequence enriched in human hot spots where it initiates recombination by its H3K4 trimethylation activity. Disruption of PRDM9 expression in mice caused sterility, and addition of PRDM9 Via baculovirus to sterile mouse hybrids rescued the sterile phenotype. Confirmation of the essential role of PRDM9 methyl transferase activity is provided by the observation that transgenic expression of catalytically active, but not inactive PRDM9 in Prdm9 null mice is sufficient to rescue the defects observed in PRDM9 null mice. In human, a handful of PRDM9 SNPs have been shown to be associated with azoospermia and sterility as well. The expression of PRDM9 in male germ cells and its role in the progression of meiosis make PRDM9 a candidate target for pharmacologic compounds that may work as non-hormonal, reversible male contraceptives.

PRDM9 has also been identified as a meiosis-specific cancer/testis gene. These genes encode cancer/testis antigens that are a group of cancer-specific biomarkers expressed in the testes of healthy adults that can also be activated in cancers. PRDM9 protein has been detected in the human testicular embryonic carcinoma cell line NTERA-2 and could potentially be used as an antigenic target in clinical applications. Studies linking PRDM9 with cancer have reported that an excess of rare PRDM9 alleles were found in children affected by B-cell precursor acute lymphoblastic leukemia (B-ALL), and their parents as well as the upregulation of PRDM9 in human ovarian cancer.

PRDM9 has a role in genetic diversity and proper progression of meiosis, and a mediation of its activity may increase the risk of certain diseases. This raises the question of whether or not variability of PRDM9 and change in its level of activity may contribute to an increased risk for other rearrangement-associated diseases.

### SUMMARY OF THE INVENTION

In one aspect, the present invention provides Compounds of Formula (I):

R¹—R²—R³—R⁴ (I)

or a pharmaceutically acceptable salt thereof, wherein:

R¹ is selected from C₁-C₆ alkyl, C₁-C₆ hydroxyalkyl, C₆-C₁₀ aryl and C₃-C₆ cycloalkyl, wherein said C₁-C₆ alkyl group and C₆-C₁₀ aryl group is unsubstituted or substituted with up to three R^{A} groups;

R² is selected from 5 or 6-membered monocyclic heteroaryl, 5 or 6-membered cycloalkenyl, heterocycloalkyl, and —CH₂SO₂—, wherein said 5 or 6-membered monocyclic heteroaryl group, said 5 or 6-membered cycloalkenyl group, and said heterocycloalkyl group is unsubstituted or substituted with up to three R^{B} groups;

R³ is selected from —NR⁵—(C₁-C₆ alkylene)-NR⁵—, —(NR⁵)ᵣ—(C₁-C₃ alkylene)ₛ-(C₃-C₇ cycloalkyl)-(5 to 16-membered heterocycloalkyl)-(C₁-C₃ alkylene)ₛ-(NR⁵)—, and —(NR⁵)ᵣ—(C₁-C₃ alkylene)ₛ-(5 to 16-membered heterocycloalkyl)-(C₁-C₃ alkylene)ₛ-(NR⁵)ᵣ—, wherein said 5 to 16-membered heterocycloalkyl group must have at least one ring nitrogen atom and wherein said 5 to 16-membered heterocycloalkyl group, said C₁-C₆ alkylene group, and said C₃-C₇ cycloalkyl group is unsubstituted or substituted with up to three R^{C} groups;

R⁴ is selected from C₆-C₁₀ aryl, 5 or 6-membered monocyclic heteroaryl, 5 or 6-membered monocyclic heterocycloalkyl, and 9 or 10-membered bicyclic heteroaryl, wherein C₆-C₁₀ aryl group, said 5 or 6-membered monocyclic heteroaryl group, said 5 or 6-membered monocyclic heterocycloalkyl group, and said 9 or 10-membered bicyclic heteroaryl group is unsubstituted or substituted with up to three R^{D} groups;

each occurrence of R⁵ is independently selected from H, C₁-C₆ alkyl, and C₃-C₆ cycloalkyl;

each occurrence of R^{A} is independently selected from C₁-C₆ alkyl, C₁-C₆ alkylamino, —O—(C₁-C₆ alkyl), —O—(C₃-C₆ cycloalkyl), —O—(C₃-C₆ monocyclic cycloalkyl), halo, —OH, C₁-C₆ haloalkyl, C₁-C₆ hydroxyalkyl, —CN, —O—(C₁-C₆ haloalkyl), —C(O)O—(C₁-C₆ alkyl), —C(O)N(R⁵)₂, —(C₁-C₆ alkyl)-N(R⁵)₂, and —N(R⁵)₂;

each occurrence of R^{B} is independently selected from C₁-C₆ alkyl, halo, phenyl, and 5 or 6-membered monocyclic heteroaryl, wherein said phenyl group is unsubstituted or substituted with up to three groups, each independently selected from C₁-C₆ alkyl, —O—(C₁-C₆ alkyl), halo, and —N(R⁵)₂;

each occurrence of R^{C} is independently selected from C₁-C₆ alkyl, —O—(C₁-C₆ alkyl), —(C₁-C₃ alkylene)-O—(C₁-C₅ alkyl), C₁-C₆ haloalkyl, C₁-C₆ hydroxyalkyl, —SO₂—(C₁-C₆ alkyl), phenyl, halo, —CN, —OH and —C(O)N(R⁵)₂;

each occurrence of R^{D} is independently selected from C₁-C₆ alkyl, —O—(C₁-C₆ alkyl), —O—(C₃-C₆ cycloalkyl), —O—(C₃-C₆ monocyclic cycloalkyl), halo, —OH, C₁-C₆ haloalkyl, C₁-C₆ hydroxyalkyl, —CN, —O—(C₁-C₆ haloalkyl), —C(O)O—(C₁-C₆ alkyl), —C(O)N(R⁵)₂ and —N(R⁵)₂;

each occurrence of r is independently 0 or 1; and

each occurrence of s is independently 0 or 1; and

The Compounds of Formula (I) (also referred to herein as the “Substituted Heterocyclic Compounds”), and pharmaceutically acceptable salts or prodrugs thereof can be useful, for example, as inhibitors of PRDM9 activity, and for the treatment of diseases and conditions associated with PRDM9 activity.

Accordingly, the present invention provides methods for the inhibition of PRDM9 activity in a subject, comprising administering to the subject an effective amount of at least one Substituted Heterocyclic Compound. In addition, the present invention provides methods for the treatment of diseases and conditions associated with PRDM9 activity in a subject, comprising administering to the subject an effective amount of at least one Substituted Heterocyclic Compound.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention includes Substituted Heterocyclic Compounds, compositions comprising at least one Substituted Heterocyclic Compound, and methods of using the Substituted Heterocyclic Compounds for inhibition of PRDM9 activity and for the treatment of diseases and conditions associated with PRDM9 activity.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of“hydroxyalkyl,” “haloalkyl,” “-O-alkyl,” etc.

As used herein, and throughout this disclosure, the following terms, unless otherwise indicated, shall be understood to have the following meanings:

A “subject” is a human or non-human mammal. In one embodiment, a subject is a human. In another embodiment, a subject is a primate. In another embodiment, a subject is a monkey. In another embodiment, a subject is a chimpanzee. In still another embodiment, a subject is a rhesus monkey.

The term “effective amount” as used herein, refers to an amount of Substituted Heterocyclic Compound and/or an additional therapeutic agent, or a composition thereof that is effective in inhibiting PRDM9 activity and in producing the desired therapeutic, ameliorative, inhibitory or preventative effect when administered to a subject suffering from a disease mediated by PRDM9 in a subject.

In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to a disease mediated by PRDM9, refers to reducing the likelihood or severity of said disease.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. The term “C₂-C₆ alkenyl” refers to an alkenyl group having from 2 to 6 carbon atoms. The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. The term “C₂-C₆ alkynyl” refers to an alkynyl group having from 2 to 6 carbon atoms. The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group has from about 3 to about 5 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms. The term “C₂-C₄ alkylene” refers to an alkylene group having from 2 to 4 carbon atoms.

The term “alkenylene,” as used herein, refers to an alkenyl group, as defined above, wherein one of the alkenyl group's hydrogen atoms has been replaced with a bond. Non-limiting examples of alkenylene groups include —CH═CH—, —CH═CHCH₂—, —CH₂CH═C—, —CH₂CH═CHCH₂—, —CH═CHCH₂CH₂—, —CH₂CH₂CH═CH— and —CH(CH₃)CH═CH—. In one embodiment, an alkenylene group has from 2 to about 6 carbon atoms. In another embodiment, an alkenylene group has from about 3 to about 5 carbon atoms. In another embodiment, an alkenylene group is branched. In another embodiment, an alkenylene group is linear. The term “C₂-C₆ alkylene” refers to an alkenylene group having from 2 to 6 carbon atoms. The term “C₃-C₅ alkenylene” refers to an alkenylene group having from 3 to 5 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. In one embodiment, an aryl group can be optionally fused to a cycloalkyl or cycloalkanoyl group. Non-limiting examples of aryl groups include phenyl and naphthyl. In one embodiment, an aryl group is phenyl. The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group is unsubstituted or substituted with one or more “ring system substituents” which may be the same or different, and are as defined herein below. In one embodiment, a cycloalkyl group is unsubstituted. The term “3 to 7-membered cycloalkyl” refers to a cycloalkyl group having from 3 to 7 ring carbon atoms. Unless otherwise indicated, a cycloalkyl group is unsubstituted. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

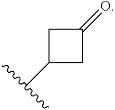 [SMILES: *C1CC(=O)C1]

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂C₁ and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms have been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. In another embodiment, a heteroaryl group is bicyclic and has 9 or 10 ring atoms. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered monocyclic heteroaryl. In another embodiment, a heteroaryl group comprises a 5- to 6-membered monocyclic heteroaryl group fused to a benzene ring.

The term “heterocycloalkyl,” as used herein, refers to a non-aromatic saturated monocyclic or multicyclic ring system comprising 3 to about 16 ring atoms, wherein one or more of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from about 3 to about 7 ring atoms. In another embodiment, a heterocycloalkyl group is monocyclic, bicylic or tricyclic and has 5 to 16 ring atoms (“5 to 16-membered heterocycloalkyl”). In another embodiment, a heterocycloalkyl group is monocyclic has 5 to or 6 ring atoms (“5 or 6-membered monocyclic heterocycloalkyl”). In another embodiment, a heterocycloalkyl group is“multicyclic” (bicyclic or tricyclic), and has from about 8 to about 18 ring atoms (“8 to 16-membered multicyclic heterocycloalkyl”). In another embodiment, a heterocycloalkyl group is“multicyclic” (bicyclic or tricyclic), and has from about 8 to about 18 ring atoms (“8 to 16-membered multicyclic heterocycloalkyl”). In another embodiment, a heterocycloalkyl group is bicyclic and has from about 8 to about 14 ring atoms. In another embodiment, a heterocycloalkyl group is bicyclic and has from about 8 to about 11 ring atoms. In one embodiment, a heterocycloalkyl group is monocyclic. In another embodiment, a heterocycloalkyl group is bicyclic. In another embodiment, a heterocycloalkyl group is tricyclic. The multicyclic heterocycloalkyl groups may have their component rings joined via various chemical bonding, including but not limited to, fused ring systems, spirocyclic ring systems, bridged ring systems and biphenyl-type heterocycloalkyl ring systems. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone and the like, and all isomers thereof. Non-limiting examples of multicyclic heterocycloalkyl rings include the following:

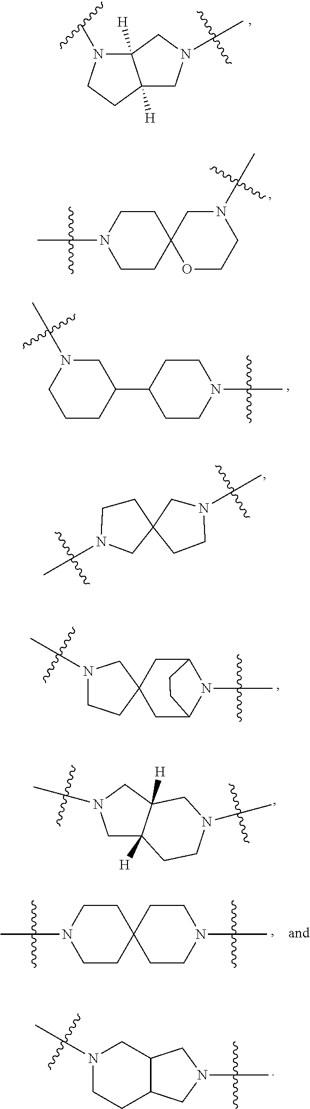

A ring carbon atom of a heterocycloalkyl group may be functionalized as a carbonyl group. An illustrative example of such a heterocycloalkyl group is:

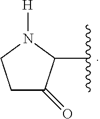 [SMILES: *C1NCCC1=O]

The term “heterocycloalkenyl,” as used herein, refers to an heterocycloalkyl group, as defined above, which is non-aromatic and contains at least one endocyclic double bond between two adjacent ring atoms. A heterocycloalkenyl group can be joined via a ring carbon, ring silicon atom or ring nitrogen atom. In one embodiment, a heterocycloalkenyl group is monocyclic and has from about 3 to about 7 ring atoms. In another embodiment, a heterocycloalkenyl group is monocyclic has from about 5 to about 8 ring atoms. In another embodiment, a heterocycloalkenyl group is bicyclic and has from about 8 to about 1 ring atoms. In still another embodiment, a heterocycloalkenyl group is monocyclic and has 5 or 6 ring atoms. In one embodiment, a heterocycloalkenyl group is monocyclic. In another embodiment, a heterocycloalkenyl group is bicyclic. There are no adjacent oxygen and/or sulfur atoms present in the ring system. Any —NH group in a heterocycloalkenyl ring may be substituted or may exist protected such as, for example, as an —N(BOC), —N(Cbz), —N(Tos) group and the like; such protected heterocycloalkenyl groups are considered part of this invention. The term “heterocycloalkenyl” also encompasses a heterocycloalkenyl group, as defined above, which is fused to an aryl (e.g., benzene) or heteroaryl ring. The nitrogen or sulfur atom of the heterocycloalkenyl can be optionally oxidized to the corresponding N-oxide, S-oxide or S,S-dioxide.

A ring carbon atom of a heterocycloalkenyl group may be functionalized as a carbonyl group. An illustrative example of such a heterocycloalkenyl group is:

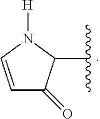 [SMILES: *C1NC=CC1=O]

In one embodiment, a heterocycloalkenyl group is a 5-membered monocyclic heterocycloalkenyl. In another embodiment, a heterocycloalkenyl group is a 6-membered monocyclic heterocycloalkenyl. The term “4 to 7-membered monocyclic heterocycloalkenyl” refers to a monocyclic heterocycloalkenyl group having from 4 to 7 ring atoms. The term “5 to 8-membered monocyclic heterocycloalkenyl” refers to a monocyclic heterocycloalkenyl group having from 5 to 8 ring atoms. The term “8 to 11-membered bicyclic heterocycloalkenyl” refers to a bicyclic heterocycloalkenyl group having from 8 to 11 ring atoms.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan. It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York. When any substituent or variable (e.g., alkyl, R¹, R⁷, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts. Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Substituted Heterocyclic Compound or a pharmaceutically acceptable salt of the compound. The transformation may occur by various mechanisms (e.g., by metabolic or chemical processes), such as, for example, through hydrolysis in blood. Similarly, if a Substituted Heterocyclic Compound contains an alcohol functional group, a prodrug can be formed by the replacement of one or more of the hydrogen atoms of the alcohol groups with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, or glycosyl (the radical resulting from the removal of a hydroxyl group of the hemiacetal form of a carbohydrate).

If a Substituted Heterocyclic Compound incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y⁵ is (C₁-C₄) alkyl and Y³ is (C₁-C₆)alkyl; carboxy (C₁-C₆)alkyl; amino(C₁-C₄)alkyl or mono-N— or di-N,N—(C₁-C₆)alkylaminoalkyl; —C(Y⁴)Y⁵ wherein Y⁴ is H or methyl and Y⁵ is mono-N— or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters, including those corresponding to both natural and non-natural amino acids (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvates, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Substituted Heterocyclic Compounds can form salts which are also within the scope of this invention. Reference to a Substituted Heterocyclic Compound herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Substituted Heterocyclic Compound contains both a basic moiety, such as, but not limited to amino, pyridine or imidazole, and an acidic moiety, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Substituted Heterocyclic Compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates), and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts, Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), arylalkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Stereochemically pure compounds may also be prepared by using chiral starting materials or by employing salt resolution techniques. Also, some of the Substituted Heterocyclic Compounds may be atropisomers (e.g., substituted biaryls), and are considered as part of this invention. Enantiomers can also be directly separated using chiral chromatographic techniques.

It is also possible that the Substituted Heterocyclic Compounds may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Substituted Heterocyclic Compound incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H), and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

The Substituted Heterocyclic Compounds are useful in human and veterinary medicine for treating or preventing diseases and conditions associated with PRDM9 activity in a subject. In one embodiment, the Substituted Heterocyclic Compounds can be inhibitors of PRDM9 activity. Accordingly, the Substituted Heterocyclic Compounds are useful for treating diseases and conditions associated with PRDM9 activity. In accordance with the invention, the Substituted Heterocyclic Compounds can be administered to a subject in need of treatment or prevention of a disease or condition associated with PRDM9 activity.

Accordingly, in one embodiment, the invention provides methods for treating or a disease or condition associated with PRDM9 activity in a subject in a subject comprising administering to the subject an effective amount of at least one Substituted Heterocyclic Compound or a pharmaceutically acceptable salt thereof. In one embodiment, the present invention provides methods for treating cancer in a subject comprising administering to the subject an effective amount of at least one Substituted Heterocyclic Compound or a pharmaceutically acceptable salt thereof.

### LIST OF ABBREVIATIONS

Anal.=analytical
ACN=acetonitrile
BH₃DMS=borane dimethylsulfide complex
BH₃.THF=borane tetrahydrofuran complex
BINAP=(2,2′-bis(diphenylphosphino)-1,1′-binaphthyl)
BrettPhos G3=[(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
t-BuOK=potassium tert-butoxide
t-BuONa=sodium tert-butoxide
br=broad
calc.=calculated
CDI=carbonyldiimidazole
d=doublet
DBU=1,8-Diazabicyclo[5.4.0]undec-7-ene
DCE=1,2-dichloro ethane
DCM=dichloromethane
DIPEA or DIEA=N,N-diisopropylethylamine
DME=dimethoxyethane
DMF=dimethylformamide
DMSO=dimethyl sulfoxide
dppf=1,1′-ferrocenediyl-bis(diphenylphosphine)
DTT=dithiothreitol
EDC.HCl=N-(3-dimethylaminopropyl)-N′-ethylcarbodiimide hydrochloride
ESI electrospray ionization
Et₂O=diethylether
Et₃N or TEA=triethylamine
EtOAc=ethyl acetate
EtOH=ethanol
HATU=-[Bis(dimethylamino)methylene]-1H-1,2,3-triazolo[4,5-b]pyridinium 3-oxid hexafluorophosphate
HCl=hydrochloric acid
HOBt=hydroxybenzotriazole
HPLC=high-pressure liquid chromatography
HTP=High Throughput Purification
IPA=iso-propyl alcohol
KOt-Bu=potassium tert-butoxide
LCMS=liquid chromatography-mass spectrometry
m=multiplet
mCPBA=m-chloroperbenzoic acid
MeCN=acetonitrile
MeI=iodomethane
MeOH=methyl alcohol
Me₂SO₄=dimethyl sulfate
MS=mass spectroscopy
NBS=N-bromosuccinimide
NMP=N-methylpyrrolidine
NMR=nuclear magnetic resonance spectroscopy
Pd/C=palladium on carbon
PdCl₂=palladium(II) chloride
Pd₂(dba)₃=tris(dibenzylideneacetone)dipalladium(0)
Pd(PPh₃)₄=tetrakis(triphenylphosphine)palladium(0)
PE=petroleum ether
rt=room temperature
RuPhos G3=(2-Dicyclohexylphosphino-2′,6′-diisopropoxy-1,1′-biphenyl)[2-(2′-amino-1,1′-biphenyl)]palladium(II) methanesulfonate
s=singlet
SiO₂=silical gel
SAM S-(5′adenosyl)-L-methionine
t=triplet
TFA=trifluoroacetic acid
TFAA=trifluoroacetic anhydride
TH=tetrahydrofuran
TLC=thin-layer chromatography
wt %=percentage by weight

### The Compounds of Formula (I)

The present invention provides Substituted Heterocyclic Compounds of Formula (I):

R¹—R²—R³—R⁴ (I)

and pharmaceutically acceptable salts thereof, wherein R¹, R², R³, and R⁴ are defined above for the Compounds of Formula (I).

In one embodiment, the compound of formula (I) has the formula (Ia):

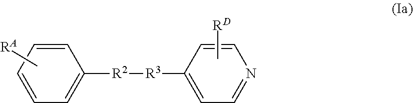

wherein:

R² is selected from oxadiazolyl, isoxazolyl, pyrazolyl, and dihydropyrrolyl, which is unsubsituted or subtituted with R^{B};

R³ is —NR⁵—(C₁-C₆ alkylene)-NR⁵—, —(NR⁵)ᵣ—(C₁-C₃ alkylene)ₛ-(5 or 6-membered monocyclic heterocycloalkyl)-(C₁-C₃ alkylene)ₛ-(NR⁵)ᵣ— or —(NR⁵)ᵣ(C₁-C₃ alkylene)ₛ-(8 to 14-membered multicyclic heterocycloalkyl)-(C₁-C₃ alkylene)ₛ-(NR⁵)ᵣ—, wherein said 5 or 6-membered monocyclic heterocycloalkyl group and said 8 to 14-membered multicyclic heterocycloalkyl group must have at least one ring nitrogen atom and wherein said 5 or 6-membered monocyclic heterocycloalkyl group and said 8 to 14-membered multicyclic heterocycloalkyl group is unsubsituted or subtituted with R^{C};

each occurrence of R⁵ is independently selected from H, methyl and cyclopropyl;

R^{A} represents up to 2 ring substituents, each independently selected from Cl, F, —(C₁-C₆ alkyl)-N(R⁵) methoxy and ethoxy;

R^{B} is independently selected from C₁-C₆ alkyl, halo, phenyl, and 5 or 6-membered monocyclic heteroaryl, wherein said phenyl group is unsubsituted or subtituted with up to three groups, each independently selected from C₁-C₆ alkyl, —O—(C₁-C₆ alkyl), halo, and —N(R⁵)₂;

R^{C} represents up to 2 ring substituents, each independently selected from C₁-C₆ alkyl, methoxy, —(C₁-C₃ alkylene)-O—(C₁-C₆ alkyl), C₁-C₆ haloalkyl, C₁-C₆ hydroxyalkyl, —SO₂—(C₁-C₆ alkyl), phenyl, halo, —CN, —OH and —C(O)N(R⁵)₂;

R^{D} represents up to 2 ring substituents, each independently selected from C₁-C₆ alkyl;

each occurrence of r is independently 0 or 1; and

each occurrence of s is independently 0 or 1.

In one embodiment, the compound of formula (I) has the formula (Ib):

R¹—R²—R³—R⁴ (Ib)

or a pharmaceutically acceptable salt thereof, wherein:

R¹ is selected from C₁-C₆ alkyl, C₁-C₆ hydroxyalkyl, C₆-C₁₀ aryl and C₃-C₆ cycloalkyl, wherein said C₁-C₆ alkyl group and C₆-C₁₀ aryl group is unsubstituted or substituted with up to three R^{A} groups;

R² is selected from 5 or 6-membered monocyclic heteroaryl, 5 or 6-membered cycloalkenyl, heterocycloalkyl, and —CH₂SO₂—, wherein said 5 or 6-membered monocyclic heteroaryl group, said 5 or 6-membered cycloalkenyl group, and said heterocycloalkyl group is unsubstituted or substituted with up to three R^{B} groups;

R³ is selected from —NR⁵—(C₁-C₆ alkylene)-NR⁵—, —(NR⁵)ᵣ—(C₁-C₃ alkylene)ₛ-(C₃-C₇ cycloalkyl)-(5 to 16-membered heterocycloalkyl)-(C₁-C₃ alkylene)ₛ-(NR⁵)ᵣ—, and —(NR⁵)ᵣ—(C₁-C₃ alkylene)ₛ-(5 to 16-membered heterocycloalkyl)-(C₁-C₃ alkylene)ₛ-(NR⁵)ᵣ—, wherein said 5 to 16-membered heterocycloalkyl group must have at least one ring nitrogen atom and wherein said 5 to 16-membered heterocycloalkyl group, said C₁-C₆ alkylene group, and said C₃-C₇ cycloalkyl group is unsubstituted or substituted with up to three R^{C} groups;

R⁴ is selected from C₆-C₁₀ aryl, 5 or 6-membered monocyclic heteroaryl, 5 or 6-membered monocyclic heterocycloalkyl, and 9 or 10-membered bicyclic heteroaryl, wherein C₆-C₁₀ aryl group, said 5 or 6-membered monocyclic heteroaryl group, said 5 or 6-membered monocyclic heterocycloalkyl group, and said 9 or 10-membered bicyclic heteroaryl group is unsubstituted or substituted with up to three R^{D} groups;

each occurrence of R⁵ is independently selected from H, C₁-C₆ alkyl, and C₃-C₆ cycloalkyl;

each occurrence of R^{A} is independently selected from C₁-C₆ alkyl, —O—(C₁-C₆ alkyl), —O—(C₃-C₆ cycloalkyl), —O—(C₃-C₆ monocyclic cycloalkyl), halo, —OH, C₁-C₆ haloalkyl, C₁-C₆ hydroxyalkyl, —CN, —O—(C₁-C₆ haloalkyl), —C(O)O—(C₁-C₆ alkyl), —C(O)N(R⁵)₂ and —N(R⁵)₂;

each occurrence of R^{B} is independently selected from C₁-C₆ alkyl, halo, phenyl, and 5 or 6-membered monocyclic heteroaryl, wherein said phenyl group is unsubsituted or subtituted with up to three groups, each independently selected from C₁-C₆ alkyl, —O—(C₁-C₆ alkyl), halo, and —N(R⁵)₂;

each occurrence of R^{C} is independently selected from C₁-C₆ alkyl, —O—(C₁-C₆ alkyl), —(C₁-C₃ alkylene)-O—(C₁-C₆ alkyl), C₁-C₆ haloalkyl, C₁-C₆ hydroxyalkyl, —SO₂—(C₁-C₆ alkyl), phenyl, halo, —CN, —OH and —C(O)N(R⁵)₂;

each occurrence of R^{D} is independently selected from C₁-C₆ alkyl, —O—(C₁-C₆ alkyl), —O—(C₃-C₆ cycloalkyl), —O—(C₃-C₆ monocyclic cycloalkyl), halo, —OH, C₁-C₆ haloalkyl, C₁-C₆ hydroxyalkyl, —CN, —O—(C₁-C₆ haloalkyl), —C(O)O—(C₁-C₆ alkyl), —C(O)N(R⁵)₂ and —N(R⁵)₂; e;

each occurrence of r is independently 0 or 1; and

each occurrence of s is independently 0 or 1.

In one embodiment, R¹ is C₁-C₆ alkyl.

In another embodiment, R¹ is C₁-C₆ hydroxyalkyl.

In another embodiment, R¹ is C₆-C₀ aryl.

In still another embodiment, R¹ is or C₃-C₆ cycloalkyl.

In another embodiment, R¹ is C₆-C₁₀ aryl wherein said C₆-C₁₀ aryl group is substituted with two R^{A} groups;

In yet another embodiment, R¹ is phenyl, which is substituted with up to 2 groups, each independently selected from halo, —(C₁-C₆ alkyl)-N(R⁵)₂, —O—(C₁-C₆ alkyl), and —C(O)O—(C₁-C₆ alkyl).

In another embodiment, R¹ is 3,5-dichloropheny.

In one embodiment, R² is 5 or 6-membered monocyclic heteroaryl.

In another embodiment, R¹ is 5 or 6-membered monocyclic heterocycloalkyl.

In another embodiment, R¹ is substituted with up to three R^{B} groups.

In still another embodiment, R² is 5-membered monocyclic heteroaryl.

In another embodiment, R² is selected from oxadiazolyl, isoxazolyl, pyrazolyl, triazolyl, and dihydropyrrolyl.

In yet another embodiment, R¹ is oxadiazolyl.

In another embodiment, R² is isoxazolyl.

In another embodiment, R¹ is pyrazolyl.

In still another embodiment, R¹ is triazolyl.

In a further embodiment, R² is dihydropyrrolyl.

In one embodiment, R³ is —NR⁵—(C₁-C₆ alkylene)-NR⁵—.

In another embodiment, R³ is —(NR⁵)ᵣ—(C₁-C₃ alkylene)ₛ-(5 or 6-membered monocyclic heterocycloalkyl)-(C₁-C₃ alkylene)ₛ-(NR⁵)ᵣ—.

In another embodiment, R³ is —NH—(C₁-C₃ alkylene)ₛ-(5 or 6-membered monocyclic heterocycloalkyl)-(C₁-C₃ alkylene)ₛ-NH—.

In still another embodiment, R³ is —(C₁-C₃ alkylene)ₛ-(5 or 6-membered monocyclic heterocycloalkyl)-(C₁-C₃ alkylene)ₛ-NH—.

In another embodiment, R³ is —NH—(C₁-C₃ alkylene)ₛ-(5 or 6-membered monocyclic heterocycloalkyl)-(C₁-C₃ alkylene)ₛ-.

In another embodiment, R³ is —(NR⁵)ᵣ—(C₁-C₃ alkylene)ₛ-(8 to 14-membered multicyclic heterocycloalkyl)-(C₁-C₃ alkylene)ₛ-(NR⁵)ᵣ.

In yet another embodiment, R³ is —NH—(C₁-C₃ alkylene)ₛ-(8 to 14-membered multicyclic heterocycloalkyl)-(C₁-C₃ alkylene)ₛ-NH—.

In another embodiment, R³ is —NH—(C₁-C₃ alkylene)ₛ-(8 to 14-membered multicyclic heterocycloalkyl)-(C₁-C₃ alkylene)ₛ-.

In a further embodiment, R³ is —(C₁-C₃ alkylene)ₛ-(8 to 14-membered multicyclic heterocycloalkyl)-(C₁-C₃ alkylene)ₛ-NH—.

In another embodiment, R³ is 8 to 14-membered bicyclic heterocycloalkyl.

In another embodiment, R³ is 10 to 14-membered tricyclic heterocycloalkyl.

In still another embodiment, R³ is a spirocyclic bicyclic heterocycloalkyl group.

In another embodiment, R³ is a fused bicyclic heterocycloalkyl group.

In another embodiment, R³ is:

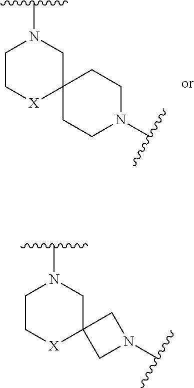

wherein X is selected from —O—, —NH, —N(CH₃)— and —CH₂—.

In one embodiment, R⁴ is C₆-C₁₀ aryl.

In another embodiment, R⁴ is 5 or 6-membered monocyclic heteroaryl.

In another embodiment, R⁴ is 5 or 6-membered monocyclic heterocycloalkyl.

In still another embodiment, R⁴ is 9 or 10-membered bicyclic heteroaryl.

In another embodiment, R⁴ is 6-membered monocyclic heteroaryl.

In another embodiment, R⁴ is optionally substituted pyridyl.

In yet another embodiment, R⁴ is:

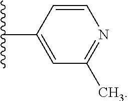 [SMILES: *c1ccnc(C)c1]

In one embodiment, variables R¹, R², R³, and R⁴ for the Compounds of Formula (I) are selected independently of each other.

In another embodiment, the Compounds of Formula (I) are in substantially purified form.

Other embodiments of the present invention include the following:

(a) A pharmaceutical composition comprising an effective amount of a Compound of Formula (I) or a pharmaceutically acceptable salt thereof, and a pharmaceutically acceptable carrier.

(b) The pharmaceutical composition of (a), further comprising a second therapeutic agent selected from the group consisting of anti-cancer agents.

(c) A pharmaceutical combination that is (i) a Compound of Formula (I), and

(ii) a second therapeutic agent selected from the group consisting of anti-cancer agents; wherein the Compound of Formula (I), and the second therapeutic agent are each employed in an amount that renders the combination effective for treating cancer.

(d) A method of inhibiting PRDM9 activity in a subject in need thereof which comprises administering to the subject an effective amount of a Compound of Formula (I).

(e) A method of treating cancer in a subject which comprises administering to the subject an effective amount of a Compound of Formula (I).

(f) The method of (g), wherein the Compound of Formula (I) is administered in combination with an effective amount of at least one second therapeutic agent selected from anti-cancer agents.

(g) A method of treating cancer in a subject in need thereof which comprises administering to the subject the pharmaceutical composition of (a), (b) or (c) or the combination of (f).

The present invention also includes a compound of the present invention for use (i) in, (ii) as a medicament for, or (iii) in the preparation of a medicament for: (a) medicine, (b) inhibiting PRDM9 activity or (c) treating cancer. In these uses, the compounds of the present invention can optionally be employed in combination with one or more second therapeutic agents selected from anti-cancer agents.

Additional embodiments of the invention include the pharmaceutical compositions, combinations and methods set forth in (a)-(g) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate. It is understood that references to compounds would include the compound in its present form as well as in different forms, such as polymorphs, solvates and hydrates, as applicable.

It is further to be understood that the embodiments of compositions and methods provided as (a) through (g) above are understood to include all embodiments of the compounds, including such embodiments as result from combinations of embodiments.

The Compounds of Formula (I) may be referred to herein by chemical structure and/or by chemical name. In the instance that both the structure and the name of a Compound of Formula (I) are provided and a discrepancy is found to exist between the chemical structure and the corresponding chemical name, it is understood that the chemical structure will predominate.

Non-limiting examples of the Compounds of Formula (I) include compounds 1-247 as set forth in the Examples below, and pharmaceutically acceptable salts thereof.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below. Alternative synthetic pathways and analogous structures will be apparent to those skilled in the art of organic synthesis.

In the methods for preparing compounds of the present invention set forth in the Examples below, functional groups in various moieties and substituents (in addition to those already explicitly noted in the foregoing schemes) may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3′ edition, 1999, and 2^{nd} edition, 1.991. The protecting groups may be removed at a convenient subsequent stage using methods known in the art. Alternatively the interfering group can be introduced into the molecule subsequent to the reaction Step of concern.

One skilled in the art of organic synthesis will recognize that the synthesis of compounds with multiple reactive functional groups, such as —OH and NH₂, may require protection of certain functional groups (i.e., derivatization for the purpose of chemical compatibility with a particular reaction condition). Suitable protecting groups for the various functional groups of these compounds and methods for their installation and removal are well-known in the art of organic chemistry. A summary of many of these methods can be found in Greene & Wuts, Protecting Groups in Organic Synthesis, John Wiley & Sons, 3^{rd} edition (1999).

One skilled in the art of organic synthesis will also recognize that one route for the synthesis of the Compounds of Formula (I) may be more desirable depending on the choice of appendage substituents. Additionally, one skilled in the relevant art will recognize that in some cases the order of reactions may differ from that presented herein to avoid functional group incompatibilities and thus adjust the synthetic route accordingly.

The intermediate compounds A through U, as depicted in the Examples below, may be further elaborated using methods that would be well-known to those skilled in the art of organic synthesis to make the full scope of the Compounds of Formula (I).

The starting materials used and the intermediates prepared using the methods set forth in the Examples below may be isolated and purified if desired using conventional techniques, including but not limited to filtration, distillation, crystallization, chromatography and alike. Such materials can be characterized using conventional means, including physical constants and spectral data.

### EXAMPLES

### General Methods

The following examples serve only to illustrate the invention and its practice. The examples are not to be construed as limitations on the scope or spirit of the invention. In these examples, all temperatures are degrees Celsius unless otherwise noted, and “room temperature” refers to a temperature in a range of from about 20° C. to about 25° C. Reactions sensitive to moisture or air were performed under nitrogen using anhydrous solvents and reagents. The progress of reactions and purity of products were determined using the following methods:

### HPLC/MS Reaction Monitoring Method:—

Column: Supelco Ascentis Express C18 (3.0×50 mm, 2.7 μm); Flow 1.25 mL/min; Mobile Phase A: H₂O+0.1% TFA; Mobile Phase B: ACN+0.1% TFA; Gradient Table: 0 min: 10% B, 0.8 min: 99% B, 1.99 min: 99% B, 2.0 min: 10% B, stop time 2.0 min, Post Time 1.5 minutes. Detection by UV, and MS with ESI(+).

### UPLC/MS Reaction Monitoring Method:—

Column: Supelco Ascentis Express C18 (2.1×50 mm, 2.7 μm); Flow 1.0 mL/min; Mobile Phase A: H₂O+0.05% TFA; Mobile Phase B: ACN+0.05% TFA; Gradient Table: 0 min: 5% B, 1.5 min: 98% B, 2.0 min: 98% B, stop time 2.0 min, Post Time 0.0 minutes. Detection by UV, and MS with ESI(+).

### HPLC/MS Purity Determination Method:—

Column: Supelco Ascentis Express C18 (3.0×100 mm, 2.7 μm); Flow 1.0 mL/min; Mobile Phase A: H₂O+0.1% TFA; Mobile Phase B: ACN+0.1% TFA; Gradient Table: 0 min: 10% B, 4.0 min: 99% B, 5.49 min: 99% B, 5.5 min: 10% B, stop time 5.5 min, Post Time 2.0 minutes. Detection by UV, and MS with ESI(+).

### UPLC/MS Purity Determination Method:—

Column: Supelco Ascentis Express C18 (2.1×100 mm, 2.7 μm); Flow 1.00 mL/min; Mobile Phase A: H₂O+0.05% TFA; Mobile Phase B: ACN+0.05% TFA; Gradient Table: 0 min: 5% B, 4.0 min: 98% B, 5.0 min: 98% B, stop time 5 min, Post Time 1.0 minutes. Detection by UV, and MS with ESI(+).

### Pre-QC MPLC Method (Gilson):—

Column: Waters Sunfire C18 (2.1×30 mm, 2.5 μm); Flow 0.9 mL/min; Mobile Phase A: H₂O+0.1% TFA; Mobile Phase B: ACN+0.1% TFA; Gradient Table: 0 min: 5% B, 4.0 min: 95% B, 5.7 min: 95% B, stop time 5.7 min, Post Time 0.8 minutes. Detection by UV, and MS with ESI(+).

Mass analysis was performed with electrospray ionization in positive ion detection mode. ¹H NMR spectra were recorded on Varian or Bruker instruments at 400-500 MHz. Concentration of solutions was carried out on a rotary evaporator in vacuo or by lyophilization. Flash chromatography was performed on pre-packed silica gel columns using a commercial MPLC system. Compounds described herein were synthesized as racemic mixtures unless otherwise stated in the experimental procedures.

### Synthesis of Intermediate Compounds

### Intermediate A. 5-chloro-3-(3,5-dichlorophenyl)-1,2,4-oxadiazole

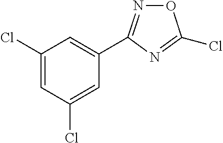 [SMILES: Clc1cc(Cl)cc(-c2noc(Cl)n2)c1]

### Step 1:

Hydroxylamine hydrochloride (8.1 g, 116.3 mmol) was added to a stirred mixture of 3,5-dichlorobenzonitrile (10 g, 58.1 mmol), K₂CO₃ (12.1 g, 87.2 mmol) in MeOH (70 mL) at 0-5° C. and the reaction was allowed to stir at 100° C. for 18 hours. The solvent was evaporated, and the resulting residue was taken up in a 1:4 mixture of water and chloroform. The organic layer was separated, washed with water and brine, dried over Na₂SO₄, filtered, and concentrated in vacuo to provide a crude residue. The residue, a solid, was not purified, and the crude material moved forward to the next step. MS ESI calc'd. for (C₇H₆C₁₂N₂O) [M+H]⁺ 204.99, found 204.9.

### Step 2:

Ethyl carbonochloridate (29.4 g, 271 mmol) was added to a stirred mixture of (Z)-3,5-dichloro-N-hydroxybenzimidamide (50 g, 244 mmol) in Pyridine (500 mL) at 0-5° C., and the reaction was heated to 100° C., and allowed to stir at this temperature for 18 hours. The reaction mixture was cooled to room temperature, then concentrated in vacuo to provide a crude residue, which was diluted with ethyl acetate (200 mL), washed with water (200 mL), dried (Na₂SO₄), filtered, and the filtrate was concentrated in vacuo. The resulting residue was recrystallized from ethyl acetate: petroleum ether (10:1) (300 mL) and the collected solid product was dried in vacuo at room temperature to provide 3-(3, 5-dichlorophenyl)-1,2, 4-oxadiazol-5(4H)-one. ¹H NMR (400 MHz, DMSO-d₆) δ 8.23 (s, 1H), 8.07 (s, 2H).

### Step 3:

Phosphoryl trichloride (332 mg, 2.164 mmol) was added dropwise to a stirred mixture of 3-(3,5-dichlorophenyl)-1,2,4-oxadiazol-5(4H)-one (500 mg, 2.164 mmol), pyridine (171 mg, 2.164 mmol) in toluene (5 mL) at 0-5° C., and the reaction was heated to 100° C. and allowed to stir at this temperature for 8 hours. The reaction mixture was cooled to room temperature, concentrated in vacuo, and diluted with ethyl acetate (20 mL). The resulting solution was washed with water (20 mL), and brine (saturated, 30 mL), dried (Na₂SO₄), filtered, and concentrated in vacuo, and the resulting product was used without further purification. 1H NMR (400 MHz, DMSO-d₆) 37.92-7.93 (m, 3H).

The intermediates in the following Table were prepared according to the method described for intermediate A. All intermediates in the table below were used as crude products without characterization.

| Intermediate | Structure | [M + H]+ Obs'd |
|---|---|---|
| B | 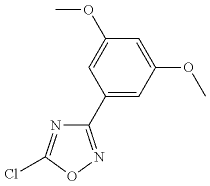 [SMILES: COc1cc(OC)cc(-c2noc(Cl)n2)c1] | Not isolated |
| C | 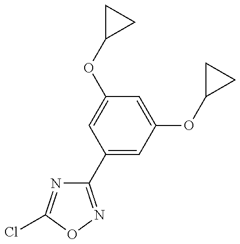 [SMILES: Clc1nc(-c2cc(OC3CC3)cc(OC3CC3)c2)no1] | Not isolated |
| D | 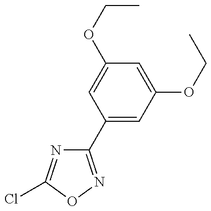 [SMILES: CCOc1cc(OCC)cc(-c2noc(Cl)n2)c1] | Not isolated |
| E | 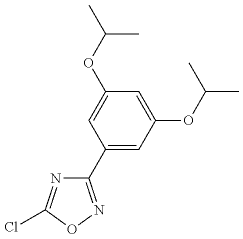 [SMILES: CC(C)Oc1cc(OC(C)C)cc(-c2noc(Cl)n2)c1] | Not isolated |

### Intermediate F. 3-bromo-5-(3,5-dimethoxyphenyl)-1,2,4-oxadiazole

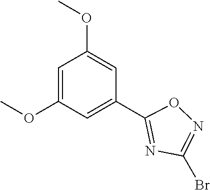 [SMILES: COc1cc(OC)cc(-c2nc(Br)no2)c1]

Hydroxycarbonimidic dibromide (1 g, 4.93 mmol) was added portion-wise to a suspension of 3,5-dimethoxybenzonitrile (1.609 g, 9.86 mmol), and sodium bicarbonate (1.243 g, 14.79 mmol) in toluene (20 mL) at 90° C. After the addition was complete, the mixture was allowed to stir at 90° C. for 3 hours. After cooling to room temperature, the mixture was diluted with EtOAc (30 mL). The organic layer was washed with H₂O (20 mL). The water layer was extracted with EtOAc (30 mL×2). The collected organic layers were washed with brine (50 mL), and dried over anhydrous Na₂SO₄. The reaction mixture was filtered, and the filtrate was concentrated in vacuo to provide a residue as yellow liquid. The residue was purified using Prep-HPLC (Column: Waters Xbridge Prep OBD C18 150×30 Sum; condition: water (0.05% ammonia hydroxide v/v)-ACN; begin: B, 49%, end: B, 69%. Gradient time (min): 10, 100% B hold time (min): 2; flow rate (ml/min): 25), then lyophilized to provide 3-bromo-5-(3,5-dimethoxyphenyl)-1,2,4-oxadiazole. ¹H NMR (400 MHz, CDCl₃) δ 7.28 (d, J=1.6 Hz, 2H) 6.72 (t, J=2.4 Hz, 1H) 3.88 (s, 6H).

The intermediates in the following table were prepared according to the method described for intermediate 6.

| Intermediate | Structure | HNMR |
|---|---|---|
| G | 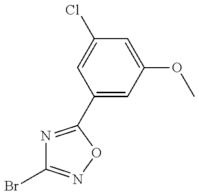 | ¹H NMR (400 MHz, CDCl₃) δ 7.72 (t, J = 1.6 Hz, 1 H), 7.53 (dd, J = 2.3, 1.4 Hz, 1 H), 7.16 (t, J = 2.1 Hz, 1 H), 3.90 (s, 3 H). |
| H | 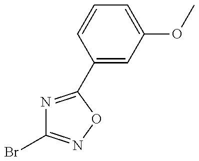 | ¹H NMR (400 MHz, CDCl₃) δ 7.71-7.75 (m, 1 H), 7.62-7.63 (m, 1 H), 7.46 (t, J = 8.0 Hz, 1 H), 7.16-7.20 (m, 1 H), 3.90 (s, 3 H). |
| I | 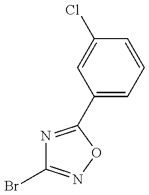 [SMILES: Clc1cccc(-c2nc(Br)no2)c1] | ¹H NMR (400 MHz, CDCl₃) δ 8.14 (t, J = 1.6 Hz, 1 H), 8.03 (dt, J = 7.7, 1.3 Hz, 1 H), 7.60- 7.65 (m, 1 H), 7.49-7.54 (m, 1 H). |

### Intermediate J. 9-benzyl 4-(tert-butyl) 1,4,9-triazaspiro[5.5]undecane-4,9-dicarboxylate

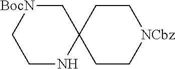 [SMILES: CC(C)(C)OC(=O)N1CCNC2(CCN(C(=O)OCc3ccccc3)CC2)C1]

### Step 1:

To a stirred solution of benzyl 4-oxopiperidine-1-carboxylate (18 g, 77.16 mmol), ethane-1,2-diamine (5.57 g, 93 mmol), and benzyltriethylammonium chloride (0.88 g, 3.86 mmol) in CHCl₃(300 mL) was added sodium hydroxide (15.43 g, 193 mmol) (50% in water) dropwise at 0° C. After the addition, the reaction was allowed to stir at 30° C. for 16 hours. Water (200 mL) was added, and the aqueous layer was extracted with DCM (3×50 mL). The organic layers are combined, dried over anhydrous magnesium sulfate, filtered, and concentrated in vacuo to provide a crude product which was purified using flash silica gel chromatography (ISCO®; 20 g SepaFlash® Silica Flash Column, eluent of [0-10]% MeOH/EtOAc gradient @40 mL/min) to provide benzyl 5-oxo-1,4,9-triazaspiro[5.5]undecane-9-carboxylate. MS ES calc'd for C₁₆H₂₂N₃O₃ [M+H]⁺ 304.1, found 304.1. ¹H NMR (400 MHz, CD₃OD) δ 7.27-7.39 (m, 5H), 5.12 (s, 2H), 3.92 (d, J=13.1 Hz, 2H), 3.24-3.33 (m, 4H), 2.98 (t, J=5.5 Hz, 2H), 1.90-2.09 (m, 2H), 1.69 (s, 2H).

### Step 2:

BH₃.DMS (26.9 mL, 283 mmol) was added to a stirred mixture of benzyl 5-oxo-1,4,9-triazaspiro[5.5]undecane-9-carboxylate (8.6 g, 28.3 mmol) in THF (140 mL), and the reaction was allowed to stir at room temperature around 20° C. for 24 hours. The reaction mixture was quenched with MeOH (100 mL), and the solvent was removed in vacuo to provide a crude residue, which was dissolved in MeOH (100 mL), and the reaction was allowed to stir at 80° C. for 6 hours. HCl/MeOH (4 N, 80 mL) was added, and the reaction mixture was allowed to stir at 60° C. for 1 hour. MeOH was removed in vacuo to provide benzyl 1,4,9-triazaspiro[5.5]undecane-9-carboxylate, which Was used without further purification. LCMS (ESI) calc'd for C₁₆H₂₃N₃O₂ [M+H]⁺: 290.2, found: 290.1.

### Step 3:

Boc₂O (5.18 mL, 22.32 mmol) was added to a stirred mixture of benzyl 1,4,9-triazaspiro[5.5]undecane-9-carboxylate (10.3 g, 20.29 mmol), and Et₃N (8.48 mL, 60.9 mmol) in DCM (100 mL), and the reaction was allowed to stir at room temperature for 1 hour. TLC on silica showed the starting material was consumed, and a new product was formed. The solvent was evaporated in vacuo to provide a crude residue which was purified using flash silica gel chromatography (ISCO; 40 g Agela Silica Flash Column, Eluent of 0-50% EtOAc/petroleum ether gradient @ 30 mL/min) to provide 9-benzyl 4-tert-butyl 1,4,9-triazaspiro[5.5]undecane-4,9-dicarboxylate. ¹H NMR (400 MHz, CDCl₃) δ 7.37-7.30 (m, 5H), 5.12 (s, 2H), 3.67-3.08 (m, 8H), 2.84 (s, 2H), 1.56-1.42 (m, 13H).

### Intermediate K. 9-(2-methylpyridin-4-yl)-2,9-diazaspiro[5.5]undecane

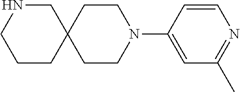 [SMILES: Cc1cc(N2CCC3(CCCNC3)CC2)ccn1]

### Step 1:

4-fluoro-2-methylpyridine (19.36 mg, 0.174 mmol) was added to a mixture of tert-butyl 2,9-diazaspiro[5.5]undecane-2-carboxylate oxalate (50 mg, 0.145 mmol), and TEA (0.081 mL, 0.581 mmol) in 1-butanol (2 mL) at 26° C. The reaction mixture was allowed to stir at 110° C. for 15 hours. LCMS showed most of the starting material was consumed. TLC (SiO₂, R_{f}=0.35, DCM:MeOH=10:1). After cooling to room temperature, the mixture was concentrated in vacuo to provide a solid, which was dissolved in H₂O (10 mL), extracted with DCM/MeOH (10/1, v/v) (10 mL×3). The organic layers were washed with brine (10 mL), and dried over anhydrous Na₂SO₄. The reaction mixture was filtered, and the filtrate was concentrated in vacuo to provide a residue as a light yellow liquid. The residue was purified using prep-TLC (SiO₂, DCM:MeOH=10:1) to provide tert-butyl 9-(2-methylpyridin-4-yl)-2,9-diazaspiro[5.5]undecane-2-carboxylate. MS ESI calcd. for C₂₀H₃₂N₃O₂ [M+H]⁺, 346.2, found, 346.0. ¹H NMR (400 MHz, CD₃OD): δ 7.96 (d, J=6.3 Hz, 1H), 6.72 (d, 1H), 6.68 (dd, J=6.3, 2.4 Hz, 1H), 3.51-3.59 (m, 2H), 3.32-3.45 (m, 6H), 2.39 (s, 3H), 1.50-1.62 (m, 8H), 1.46 (s, 9H).

### Step 2:

A mixture of tert-butyl 9-(2-methylpyridin-4-yl)-2,9-diazaspiro[5.5]undecane-2-carboxylate (61 mg, 0.177 mmol) in a co-solvent of TFA (0.3 mL), and DCM (3 mL) was allowed to stir at 23° C. for 1 hour. LCMS showed the starting material was consumed. The reaction mixture was concentrated in vacuo to provide 9-(2-methylpyridin-4-yl)-2,9-diazaspiro[5.5]undecane as a yellow oil, which was used without further purification directly without further purification. MS EST calcd. for C₁₅H₂₄N₃ [M+H]⁺, 246.2, found, 246.1.

### Intermediate L. (E)-N′-hydroxy-9-(2-methylpyridin-4-yl)-2,9-diazaspiro[5.5]undecane-2-carboximidamide

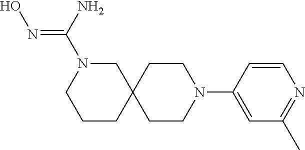 [SMILES: Cc1cc(N2CCC3(CCCN(/C(N)=N/O)C3)CC2)ccn1]

### Step 1:

NaH (391 mg, 9.78 mmol) (60% in mineral oil) was added portion-wise to a mixture of 9-(2-methylpyridin-4-yl)-2,9-diazaspiro[5.5]undecane (300 mg, 1.223 mmol, 37.8% content) in THF (10 mL) at 0° C.; the mixture was allowed to stir at 0° C. for 30 minutes. Cyanic bromide (194 mg, 1.834 mmol) was added portion-wise to this mixture at 0° C. After the addition was complete, the resulting mixture was allowed to warm to 0° C., and stirred for 2 hours. The reaction mixture was quenched with saturated aq. NH₄Cl (10 mL), extracted with DCM/MeOH (10/I, v/v, 10 mL×3), and the combined organic layers were washed with brine (10 mL) then dried over anhydrous Na₂SO₄. The reaction mixture was filtered, and the filtrate was concentrated in vacuo to provide 9-(2-methylpyridin-4-yl)-2,9-diazaspiro[5.5]undecane-2-carbonitrile, which was used without further purification directly without further purification. MS ESI calcd. for C₁₆H₂₃N₄ [M+H]f, 271.2, found, 271.1.

### Step 2:

Hydroxylamine hydrochloride (37.0 mg, 0.533 mmol) was added to a mixture of 9-(2-methylpyridin-4-yl)-2,9-diazaspiro[5.5]undecane-2-carbonitrile (144 mg, 0.533 mmol) in EtOH (5 mL) at 17° C. After the addition was complete, the mixture was allowed to stir at 17° C. for 15 hours. LCMS showed most of the starting material was consumed. The reaction mixture was concentrated in vacuo to provide (E)-N′-hydroxy-9-(2-methylpyridin-4-yl)-2,9-diazaspiro[5.5]undecane-2-carboximidamide, which was used without further purification directly without further purification. MS ESI calcd. for C₁₆H₂₆N₅O [M+H]⁺, 304.2, found, 304.1.

### Intermediate M. methyl 3,5-bis(difluoromethoxy)benzoate

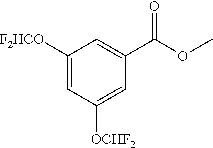 [SMILES: COC(=O)c1cc(OC(F)F)cc(OC(F)F)c1]

A mixture of methyl 3,5-dihydroxybenzoate (200 mg, 1.189 mmol), K₂CO₃ (362 mg, 2.62 mmol), and KI (1.974 mg, 0.012 mmol) in DMF (5 mL) was allowed to stir at 80° C. for 1 hour. The reaction mixture was cooled to 60° C., methyl 2-chloro-2,2-difluoroacetate (430 mg, 2.97 mmol) was slowly added dropwise at 60° C., and the reaction was allowed to stir at 60° C. for 30 minutes. The resulting mixture was heated to 80° C., and stirred for 3 hours. After cooling to room temperature, the mixture was concentrated in vacuo to provide a residue, which was diluted with EtOAc (10 mL). The organic layer was washed with H₂O (10 mL). The water layer was extracted with EtOAc (10 mL×2). The collected organic layers were washed with brine (10 mL), dried over anhydrous Na₂SO₄. The reaction mixture was filtered, and the filtrate was concentrated in vacuo to provide a residue as a yellow liquid. The residue was purified using prep-TLC (SiO₂, petroleum ether:EtOAc=3:1) to provide methyl 3,5-bis(difluoromethoxy)benzoate. ¹H NMR (400 MHz, CDCl₃): δ 7.66 (d, J=1.7 Hz, 2H), 7.12 (t, 1H), 6.38-6.76 (m, 2H), 3.95 (s, 3H).

### Intermediate N. 1-(2-methoxyethyl)-9-(2-methylpyridin-4-yl)-1,4,9-triazaspiro[5.5]undecane

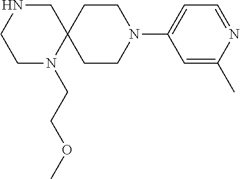 [SMILES: COCCN1CCNCC12CCN(c1ccnc(C)c1)CC2]

### Step 1:

A solution of 9-benzyl 4-tert-butyl 1,4,9-triazaspiro[5.5]undecane-4,9-dicarboxylate (2 g, 5.13 mmol), 1-bromo-2-methoxyethane (7.14 g, 51.3 mmol), and K₂CO₃ (2.129 g, 15.40 mmol) in DMF (15 mL) was allowed to stir at 80° C. for 12 hours. The reaction mixture was then diluted with water (50 mL), and EtOAc (50 mL). The organic layer was separated, and the aqueous was re-extracted with EtOAc (50 mL×3). The combined organic layers were washed with brine (100 mL), dried over anhydrous Na₂SO₄, filtered, and concentrated in vacuo to provide a crude residue, which was purified using flash silica gel chromatography (ISCO®; 20 g SepaFlash® Silica Flash Column, eluent of 30% ethyl acetate/petroleum ether gradient @ 35 mL/min) to provide 9-benzyl 4-(tert-butyl) 1-(2-methoxyethyl)-1,4,9-triazaspiro[5.5]undecane-4,9-dicarboxylate. ¹H NMR (400 MHz, CD₃OD) δ 7.27-7.40 (m, 5H), 5.12 (s, 2H), 4.00 (d, J=13.6 Hz, 2H), 3.44 (s, 4H), 3.38 (t, J=5.9 Hz, 2H), 3.29-3.34 (m, 5H), 2.65-2.73 (m, 2H), 2.56 (t, J=5.9 Hz, 2H), 1.66 (t, J=10.3 Hz, 2H), 1.45 (s, 12H).

### Step 2:

To a solution of 9-benzyl 4-tert-butyl 1-(2-methoxyethyl)-1,4,9-triazaspiro[5.5]undecane-4,9-dicarboxylate (1.3 g, 2.90 mmol) in EtOAc (20 mL) was added Pd—C(1 g, 0.940 mmol) under N₂ atmosphere. The reaction mixture was degassed and backfilled with H₂ (three times). The resulting mixture was allowed to stir under 15 psi of H₂ at 30° C. for 3 hours. TLC showed that a new spot formed, the catalyst was filtered off, and filtrate was concentrated in vacuo to provide tert-butyl 1-(2-methoxyethyl)-1,4,9-triazaspiro[5.5]undecane-4-carboxylate, which was used in next step directly. ¹H NMR (400 MHz, CD₃OD) δ 3.37-3.51 (m, 6H), 3.32-3.34 (m, 3H), 2.87-2.98 (m, 2H), 2.62-2.80 (m, 6H), 1.60-1.74 (m, 2H), 1.46 (s, 11H).

### Step 3:

A solution of tert-butyl 1-(2-methoxyethyl)-1,4,9-triazaspiro[5.5]undecane-4-carboxylate (800 mg, 2.55 mmol), 4-chloro-2-methylpyridine (651 mg, 5.10 mmol), and N-ethyl-N-isopropylpropan-2-amine (990 mg, 7.66 mmol) in NMP (10 mL) was allowed to stir at 160° C. for 3 hours. LCMS showed that the reaction was completed. Then reaction mixture was purified using Prep-HPLC to provide tert-butyl 1-(2-methoxyethyl)-9-(2-methylpyridin-4-yl)-1,4,9-triazaspiro[5.5]undecane-4-carboxylate. MS ESI calc'd. for C₂₂H₃₇N₄O₃ [M+H]⁺, 405.5, found, 405.2. Method: Column: Phenomenex Synergi C18 150×30 mm×4 um; condition: water (0.1% TFA)-ACN; begin B: 5%; end B 40%. Gradient time (min): 11; 100% B hold time (min): 2; flow rate (ml/min): 25.

### Step 4:

A mixture of tert-butyl 1-(2-methoxyethyl)-9-(2-methylpyridin-4-yl)-1,4,9-triazaspiro[5.5]undecane-4-carboxylate (700 mg, 1.730 mmol) in HCl/EtOAc (20 mL) was allowed to stir at 30° C. for 3 hours. The TLC showed the starting material was consumed, and the reaction was concentrated in vacuo to provide 1-(2-methoxyethyl)-9-(2-methylpyridin-4-yl)-1,4,9-triazaspiro[5.5]undecane, which was used in next step directly. ¹′H NMR (400 MHz, CD₃OD) δ 8.03-8.13 (m, 1H), 7.09 (s, 2H), 4.20 (d, J=14.3 Hz, 2H), 3.79-4.06 (m, 5H), 3.48-3.75 (m, 7H), 3.39-3.44 (m, 3H), 2.55 (s, 3H), 2.45 (s, 4H).

The intermediates in the following Table were prepared according to the method described for intermediate N.

| Intermediate | Structure | HNMR |
|---|---|---|
| O | 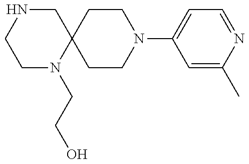 [SMILES: Cc1cc(N2CCC3(CC2)CNCCN3CCO)ccn1] | ¹H NMR (400 MHz, CD₃OD) δ 8.00-8.12 (m, 1H), 7.10 (s, 2H), 4.21 (d, J = 14.1 Hz, 2H), 3.85-4.06 (m, 6H), 3.70 (t, J = 5.3 Hz, 2H), 3.41-3.60 (m, 4H), 2.55 (s, 3H), 2.45 (s, 4H). |

### Intermediate P. 9-(2-methylpyridin-4-yl)-1-(2,2,2-trifluoroethyl)-1,4,9-triazaspiro[5.5]undecane

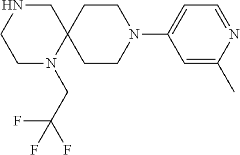 [SMILES: Cc1cc(N2CCC3(CC2)CNCCN3CC(F)(F)F)ccn1]

### Step 1:

TFAA (0.135 mL, 0.952 mmol) was added to a mixture of tert-butyl 9-(2-methylpyridin-4-yl)-1,4,9-triazaspiro[5.5]undecane-4-carboxylate (300 mg, 0.866 mmol), and Et₃N (0.362 mL, 2.60 mmol) in MeCN (5 mL), and the reaction was allowed to stir at room temperature around 15° C. for 8 hours. LCMS (ESI) showed starting material was consumed, and a new product was formed. Water (20 mL) was added, and the reaction was extracted with ethyl acetate (2×10 mL). The combined organic extracts were washed with brine (saturated, 2×20 mL), dried (Na₂SO₄), filtered, and the solvent was evaporated in vacuo to provide tert-butyl 9-(2-methylpyridin-4-yl)-1-(2,2,2-trifluoroacetyl)-1,4,9-triazaspiro[5.5]undecane-4-carboxylate, which was used, without further purification without further purification. MS ESI calc'd for C₂₁H₂₉F₃N₄O₃ [M+H]⁺: 443.2, found: 443.2.

### Step 2:

BH₃.THF (8.59 mL, 8.59 mmol) (I M in THF) was added to a mixture of tert-butyl 9-(2-methylpyridin-4-yl)-1-(2,2,2-trifluoroacetyl)-1,4,9-triazaspiro[5.5]undecane-4-carboxylate (380 mg, 0.859 mmol) in THF (2 mL), and the reaction was allowed to stir at 65° C. for 8 hours. TLC on silica showed starting material was consumed, and a new product formed. The reaction mixture was cooled to rt, MeOH (20 mL) was added, and the reaction was allowed to stir at 85° C. for 8 hours. The solvent was evaporated in vacuo to provide a residue which was purified using preparative TLC on silica gel, eluting with EtOAc/MeOH/NH₃—H2O=40:1:1 (V/V) to provide tert-butyl 9-(2-methylpyridin-4-yl)-1-(2,2,2-trifluoroethyl)-1,4,9-triazaspiro[5.5]undecane-4-carboxylate. ¹H NMR (400 MHz, CDCl₃) & 8.16 (s, 1H), 6.54-6.48 (m, 2H), 3.78 (d, J=12.3 Hz, 2H), 3.51 (s, 4H), 3.09-2.87 (m, 4H), 2.87-2.79 (m, 2H), 2.46 (s, 3H), 1.72-1.55 (m, 4H), 1.46 (s, 9H).

### Step 3:

HCl (1 mL, 4.00 mmol)(4 M in dioxane) was added to a mixture of tert-butyl 9-(2-methylpyridin-4-yl)-1-(2,2,2-trifluoroethyl)-1,4,9-triazaspiro[5.5]undecane-4-carboxylate (40 mg, 0.093 mmol) in dioxane (0.5 mL). The resulting suspension was allowed to stir at 15° C. for 16 hours. LCMS (ESI) showed starting material was consumed, and new product formed. The reaction mixture was evaporated in vacuo to provide 9-(2-methylpyridin-4-yl)-1-(2,2,2-trifluoroethyl)-1,4,9-triazaspiro[5.5]undecane, which was used without further purification. MS ESI calc'd for C₁₆H₂₃F₃N₄ [M+H]⁺: 329.2, found: 329.2.

### Intermediate Q. 9-(2-methylpyridin-4-yl)-1-phenyl-1,4,9-triazaspiro[5.5]undecane

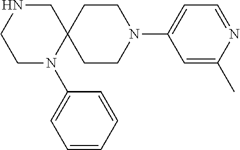 [SMILES: Cc1cc(N2CCC3(CC2)CNCCN3c2ccccc2)ccn1]

### Step 1:

Methanesulfonato(2-dicyclohexylphosphino-2′,4′,6′-tri-isopropyl-1,1′-biphenyl)(2′-amino-1,1′-biphenyl-2-yl)palladium (II) (12.22 mg, 0.014 mmol) was added to a mixture of tert-butyl 9-(2-methylpyridin-4-yl)-1,4,9-triazaspiro[5.5]undecane-4-carboxylate (50 mg, 0.144 mmol), chlorobenzene (48.7 mg, 0.433 mmol), and sodium tert-butoxide (0.144 mL, 0.288 mmol) (2M in THF) in THF (2 mL) at 21° C. in the glove box, the mixture was allowed to stir at 60° C. for 16 hours. LCMS (ESI) showed starting material remained, and new product formed. The reaction mixture was allowed to cool to room temperature and was purified using preparative HPLC (Column Waters XSELECT C18 150×30 mm×5 um. Condition: water (0.1% TFA)-ACN. Begin B 25%, end B 55%, gradient time (min): 10; 100% B hold time (min) 3.5. Flow rate (ml/min): 25.) to provide tert-butyl 9-(2-methylpyridin-4-yl)-1-phenyl-1,4,9-triazaspiro[5.5]undecane-4-carboxylate. MS ESI calc'd. for C₂₅H₃₅N₄O₂ [M+H]⁺, 423.3, found, 423.3. ¹H NMR (400 MHz, CD₃OD) δ 7.89 (d, J=7.0 Hz, 1H), 7.32-7.18 (m, 5H), 6.90-6.84 (m, 21H), 3.90-3.61 (m, 6H), 3.35 (s, 2H), 3.30 (s, 2H), 2.44 (s, 3H), 2.01-1.97 (m, 2H), 1.84-1.72 (m, 2H), 1.51 (s, 9H).

### Step 2:

HCl (1 mL, 4.00 mmol) (4 M in dioxane) was added to a mixture of tert-butyl 9-(2-methylpyridin-4-yl)-1-phenyl-1,4,9-triazaspiro[5.5]undecane-4-carboxylate (15 mg, 0.035 mmol) in dioxane (1 mL). The resulting suspension was allowed to stir at 15° C. for 12 hours. LCMS (ESI) showed starting material was consumed, and new product formed. The reaction mixture was evaporated in vacuo to provide 9-(2-methylpyridin-4-yl)-1-phenyl-1,4,9-triazaspiro[5.5]undecane, which was used without further purification. MS ESI calc'd for C₂₀H₂₅N₄ [M+H]J: 323.1, found: 323.1.

### Intermediate R. N,N-dimethyl-9-(2-methylpyridin-4-yl)-1,4,9-triazaspiro[5.5]undecane-1-carboxamide

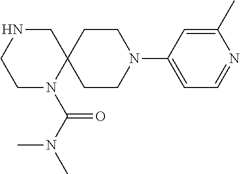 [SMILES: Cc1cc(N2CCC3(CC2)CNCCN3C(=O)N(C)C)ccn1]

### Step 1:

dimethylcarbamic chloride (62.1 mg, 0.577 mmol) was added to a mixture of tert-butyl 9-(2-methylpyridin-4-yl)-1,4,9-triazaspiro[5.5]undecane-4-carboxylate (100 mg, 0.289 mmol), and Et₃N (0.121 mL, 0.866 mmol) in MeCN (I mL), and the reaction was allowed to stir at room temperature around 60° C. for 1 hour. LCMS (ESI) showed starting material was consumed, and new product was formed. The reaction mixture was purified using preparative TLC on silica gel, eluting with DCM/MeOH(NH₃)=10:1(v/v) to provide tert-butyl 1-(dimethylcarbamoyl)-9-(2-methylpyridin-4-yl)-1,4,9-triazaspiro[5.5]undecane-4-carboxylate. MS ESI calc'd for C₂₂H₃N₅O₃ [M+H]⁺: 418.3, found: 418.3. Step2: HCl (1 mL, 4.00 mmol)(4 M in dioxane) was added to a mixture of tert-butyl 1-(dimethylcarbamoyl)-9-(2-methylpyridin-4-yl)-1,4,9-triazaspiro[5.5]undecane-4-carboxylate (35 mg, 0.084 mmol) in dioxane (0.5 mL). The resulting suspension was allowed to stir at 20° C. for 12 hours. LCMS (ESI) showed starting material was consumed, and a new product was formed. The reaction mixture was evaporated in vacuo to provide N,N-dimethyl-9-(2-methylpyridin-4-yl)-1,4,9-triazaspiro[5.5]undecane-1-carboxamide, which was used without further purification. Intermediate S. 1-methyl-9-(2-methylpyridin-4-yl)-1,4,9-triazaspiro[5.5]undecane

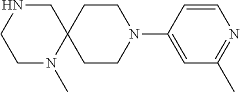 [SMILES: Cc1cc(N2CCC3(CC2)CNCCN3C)ccn1]

### Step 1:

To a solution of 9-benzyl 4-tert-butyl 1,4,9-triazaspiro[5.5]undecane-4,9-dicarboxylate (5 g, 12.84 mmol), paraformaldehyde (5.78 g, 64.2 mmol) in MeOH (100 mL) was added acetic acid (5.87 mL, 103 mmol). The reaction mixture was allowed to stir at 25° C. for 20 minutes. Then sodium triacetoxyhydroborate (21.77 g, 103 mmol) was added in portions. The reaction mixture was allowed to stir for another 12 hours. LCMS showed some starting material remained, and a new product formed. Then additional sodium triacetoxyhydroborate (10.88 g, 51.3 mmol) was added in portions. The reaction mixture was allowed to stir for another 2 hours. LCMS showed starting material was consumed, and a new product formed. The reaction mixture was quenched with H₂O (150 mL). NaHCO₃(aq.) was added until pH=7-8. The aqueous layer was extracted with DCM (100 mL×3). The combined organic layers were washed with brine (100 mL), dried over MgSO₄ and filtered. The filtrate was concentrated in vacuo to provide a residue which was purified using column chromatography (SiO₂, EtOAc/MeOH=100:1 to 20:1, v/v) to provide 9-benzyl 4-tert-butyl 1-methyl-1,4,9-triazaspiro[5.5]undecane-4,9-dicarboxylate. MS ESI calc'd for C₂₂H₃₃N₃O₄ [M+H]⁺ 404.3, found 404.3.

### Step 2:

9-benzyl 4-tert-butyl 1-methyl-1,4,9-triazaspiro[5.5]undecane-4,9-dicarboxylate (3 g, 7.43 mmol) was added to a mixture of Pd/C (0.791 g, 0.743 mmol) (10%, wet) in EtOAc (100 mL). The resulting suspension was degassed and backfilled with H₂ three times, and then stirred at 30° C. under H2 at 15 psi (hydrogen balloon) for 1 hour. LCMS (ESI) showed starting material was consumed, and a new product was formed. The reaction mixture was filtered, and solvent was removed on a rotary evaporator to provide tert-butyl 1-methyl-1,4,9-triazaspiro[5.5]undecane-4-carboxylate which was used without further purification and crude material was moved forward to the next step.

### Step 3:

Tert-butyl 1-methyl-1,4,9-triazaspiro[5.5]undecane-4-carboxylate (1.9 g, 7.05 mmol) was added to a mixture of 4-chloro-2-methylpyridine (2.249 g, 17.63 mmol), and DIPEA (3.70 mL, 21.16 mmol) in NMP (10 mL). The resulting suspension was degassed and backfilled with N₂ three times, and then stirred at 150° C. for 3 hours. LCMS (ESI) showed starting material was consumed, and a new product formed. The reaction mixture was cooled to room temperature, water (300 mL) was added, and the reaction was extracted with ethyl acetate (3×100 mL). The combined organic extracts were washed with brine (saturated, 200 mL), dried (Na₂SO₄), filtered, and the solvent was evaporated in vacuo. The residue was purified using silica gel column flash chromatography, eluting with DCM/MeOH=100:1-20:1, V/V to provide tert-butyl 1-methyl-9-(2-methylpyridin-4-yl)-1,4,9-triazaspiro[5.5]undecane-4-carboxylate. LCMS (ESI) calc'd for C₂₀H₃₂N₄O₂ [M+H]≡⁶ 361.3, found: 361.3.

### Step 4:

HCl (1.214 mL, 4.85 mmol) (4 M in dioxane) was added to a mixture of tert-butyl 1-methyl-9-(2-methylpyridin-4-yl)-1,4,9-triazaspiro[5.5]undecane-4-carboxylate (1.75 g, 4.85 mmol) in dioxane (10 mL). The resulting suspension was allowed to stir at 30° C. for 3 hours. LCMS (ESI) showed starting material was consumed, and a new product formed. The reaction mixture was evaporated in vacuo to provide 1-methyl-9-(2-methylpyridin-4-yl)-1,4,9-triazaspiro[5.5]undecane, HCl as a yellow solid which was used without further purification and the crude material moved forward to the next step.

The following intermediate was prepared according to the method described for intermediate S.

| Intermediate | Structure | [M + H]+ Obs'd |
|---|---|---|
| T | 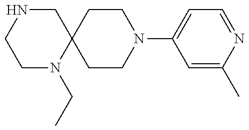 [SMILES: CCN1CCNCC12CCN(c1ccnc(C)c1)CC2] | 275.3 |

### Intermediate U. 1-methyl-9-(2-methylpyridin-4-yl)-1,4,9-triazaspiro[5.5]undecane

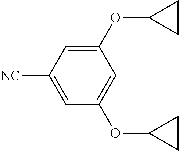 [SMILES: N#Cc1cc(OC2CC2)cc(OC2CC2)c1]

### Step 1:

To a solution of 5-bromobenzene-1,3-diol (200 mg, 1.058 mmol) in toluene (5 mL) was added Na₂CO₃ (561 mg, 5.29 mmol), chloro(1,5-cyclooctadiene)iridium(I) dimer (7.11 mg, 10.58 μmol), and vinyl acetate (638 mg, 7.41 mmol) at 30° C. The reaction mixture was allowed to stir at 100° C. for 4 hours under N₂. LCMS showed consumption of the starting material. The reaction mixture was dissolved in water (20 mL), and EtOAc (20 mL). The organic layer Was separated, and the aqueous was re-extracted with EtOAc (20 mL×3). The combined organic layers were washed with brine (20 mL), dried over anhydrous Na₂SO₄, filtered, and the filtrate was concentrated in vacuo to provide a crude residue. The residue was purified using flash silica gel chromatography (ISCO®; 4 g SepaFlash® Silica Flash Column, eluent of [0-30]% ethyl acetate/petroleum ether gradient @ 30 mL/min) to provide 1-bromo-3,5-bis(vinyloxy)benzene. ¹H NMR (400 MHz, CDCl₃) & 6.89 (d, J=2.0 Hz, 2H), 6.58-6.62 (m, 2H), 6.56 (d, J=6.0 Hz, 1H), 4.87 (d, J=1.8 Hz, 1H), 4.83 (d, J=1.8 Hz, 1H), 4.54 (dd, J=1.9, 6.1 Hz, 2H).

### Step 2:

To a solution of 1-bromo-3,5-bis(vinyloxy)benzene (180 mg, 0.747 mmol), and chloroiodomethane (790 mg, 4.48 mmol) in DCE (5 mL) was added dropwise diethylzinc (3.73 mL, 3.73 mmol) (1 M in toluene) at 0° C. The resulting mixture was allowed to stir for 1 hour at 0° C. Then the resulting mixture was allowed to stir for 16 hours at 30° C., dissolved in water (20 mL), and EtOAc (20 mL). The organic layer was separated, and the aqueous was re-extracted with EtOAc (20 mL×3). The combined organic layers were washed with brine (20 mL), dried over anhydrous Na₂SO₄, filtered, and the filtrate was concentrated in vacuo to provide a residue. The residue was purified using Prep-TLC (Silica gel, pet. ether/ethyl acetate=5/1, v/v, R_{f}=0.7) to provide 1-bromo-3,5-dicyclopropoxybenzene. ¹H NMR (400 MHz, CDCl₃) & 6.85 (d, J=2.2 Hz, 2H), 6.65 (t, J=2.0 Hz, 1H), 3.64-3.76 (m, 2H), 0.76-0.80 (m, 8H).

### Step 3:

To a solution of 1-bromo-3,5-dicyclopropoxybenzene (45 mg, 0.167 mmol) in THF (0.5 mL), and water (2 mL) was added dicyanozine (79 mg, 0.669 mmol), methanesulfonato(2-di-t-butylphosphino-2′,4′,6′-tri-isopropyl-1,1′-biphenyl)(2′-amino-1,1′-biphenyl-2-yl)palladium(II) (2.66 mg, 3.34 μmol), and 2-di-t-butylphosphino-2′,4′,6′-Triisopropylbiphenyl (1.420 mg, 3.34 μmol) at 30° C. The reaction mixture was degassed and backfilled with N₂ (three times). The reaction mixture was allowed to stir at 30° C. for 16 hours. TLC showed that the reaction had completed. The reaction mixture was dissolved in water (20 mL), and EtOAc (20 mL). The organic layer was separated, and the aqueous was re-extracted with EtOAc (20 mL×3). The combined organic layers were washed with brine (20 mL), dried over anhydrous Na₂SO₄, filtered, and the filtrate was concentrated in vacuo to provide a crude residue. The residue was purified using prep-TLC (silica gel, petroleum ether/ethyl acetate=5/1, v/v, R_{f}=0.4) to provide 3,5-dicyclopropoxybenzonitrile. ¹H NMR (400 MHz, CDCl₃) δ 6.96 (d, J=2.4 Hz, 2H), 6.89-6.93 (m, 1H), 3.72 (tt, J=3.1, 6.0 Hz, 2H), 0.80-0.84 (m, 4H), 0.75-0.80 (m, 4H).

### Example 1

### Preparation of Compound 1—(3aR,7aS)-tert-butyl 2-(3-(3,5-dichlorophenyl)-1,2,4-oxadiazol-5-yl)hexahydro-1H-pyrrolo[3,4-c]pyridine-5(6H)-carboxylate

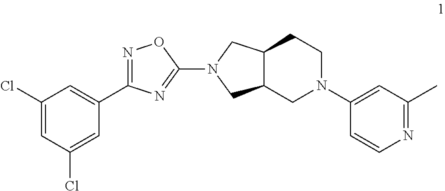 [SMILES: Cc1cc(N2CC[C@@H]3CN(c4nc(-c5cc(Cl)cc(Cl)c5)no4)C[C@@H]3C2)ccn1]

### Step 1:

An 8 mL vial was charged with 5-chloro-3-(3,5-dichlorophenyl)-1,2,4-oxadiazole (100 mg, 0.4001 mmol), (3aR,7aS)-tert-butyl hexahydro-1H-pyrrolo[3,4-c]pyridine-5(6H)-carboxylate (91 mg, 0.401 mmol), DIPEA (0.0905 mL, 0.522 mmol), and MeCN (2 mL). The reaction mixture was allowed to stir at 35° C. for 4 hours. LCMS showed the starting material was consumed. The reaction was filtered. The filtrate was purified using prep-TLC (SiO₂, petroleum ether:EtOAc=2:1) directly to provide (3aR,7aS)-tert-butyl 2-(3-(3,5-dichlorophenyl)-1,2,4-oxadiazol-5-yl)hexahydro-1H-pyrrolo[3,4-c]pyridine-5(6H)-carboxylate. MS ESI calc'd. for (C₁₆H₁₇Cl₂N₄O₃) [M+H—C₄H₈]⁺, 383.0, found, 382.9.

### Step 2:

A 40 mL vial was charged with (3aR,7aS)-tert-butyl 2-(3-(3,5-dichlorophenyl)-1,2,4-oxadiazol-5-yl)hexahydro-1H-pyrrolo[3,4-c]pyridine-5(6H)-carboxylate (90 mg, 0.204 mmol), DCM (4 mL), and a solution of hydrogen chloride (4 mL, 4 M) in 1,4-dioxane. The reaction mixture was allowed to stir at 35° C. for 1 hour. LCMS showed the starting material was consumed, and the desired mass was detected. The solvent was removed to provide a crude residue 3-(3,5-dichlorophenyl)-5-((3aS,7aS)-hexahydro-1H-pyrrolo[3,4-c]pyridin-2(3H)-yl)-1,2,4-oxadiazole. The crude was used in the next step directly. MS ESI calc'd. for (C₁₅H₁₇Cl₂N₄O) [M+H]⁺, 339.1, found, 339.0.

### Step 3:

A 40 ml vial was charged with N-ethyl-N-isopropylpropan-2-amine (45.8 mg, 0.354 mmol), 4-chloro-2-methylpyridine (22.6 mg, 0.177 mmol), 3-(3,5-dichlorophenyl)-5-((3aS,7aS)-hexahydro-1H-pyrrolo[3,4-c]pyridin-2(3H)-yl)-1,2,4-oxadiazole (60 mg, 0.177 mmol), and NMP (2 mL). The reaction mixture was allowed to stir at 160° C. for 2 hours under N₂. LCMS and TLC showed product formation. The reaction was purified using prep-HPLC (TFA) directly to provide 3-(3,5-dichlorophenyl)-5-((3aS,7aS)-5-(2-methylpyridin-4-yl)hexahydro-1H-pyrrolo[3,4-c]pyridin-2(3H)-yl)-1,2,4-oxadiazole. MS ESI calcd. for (C₂₁H₂₂Cl₂N₅O) [M+H+CH₃CN]⁺, 430.1, found, 429.8. ¹H NMR (400 MHz, CD₃CD)⁶ 7.99 (d, J=7.8 Hz, 1H), 7.86 (d, J=2.0 Hz, 2H), 7.60 (t, J=1.9 Hz, 1H), 7.08-7.03 (m, 2H), 4.00-3.90 (m, 2H), 3.85-3.78 (m, 3H), 3.66-3.52 (m, 2H), 3.39 (dd, J=6.7, 10.7 Hz, 1H), 2.83-2.72 (m, 2H), 2.52 (s, 3H), 2.06-1.96 (m, 1H), 1.79-1.67 (m, 1H).

The following compounds were prepared according to the method described in Example 1, step 1, and where appropriate, step 2 and step 3 as well.

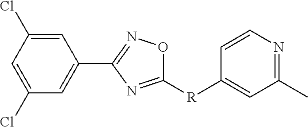

| | | [M + H]+ |
|---|---|---|
| No. | —R— | (Obs'd) |
| 2 | 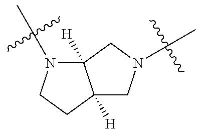 [SMILES: *N1C[C@H]2CCN(*)[C@H]2C1] | 416.1 |
| 3 | 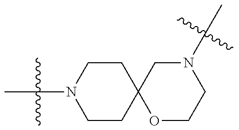 [SMILES: *N1CCC2(CC1)CN(*)CCO2] | 460.1 |
| 4 | 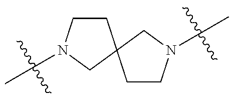 [SMILES: *N1CCC2(CCN(*)C2)C1] | 430.1 |
| 5 | 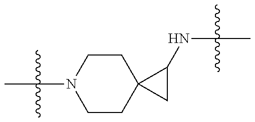 [SMILES: *NC1CC12CCN(*)CC2] | 430.1 |
| 6 | 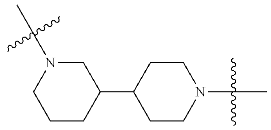 [SMILES: *N1CCC(C2CCCN(*)C2)CC1] | 472.1 |
| 7 | 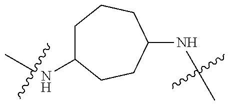 | 432.1 |
| 8 | 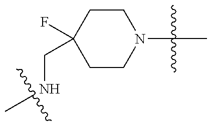 | 436.1 |
| 9 | 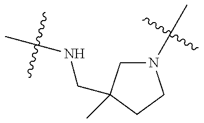 [SMILES: *NCC1(C)CCN(*)C1] | 418.1 |
| 10 | 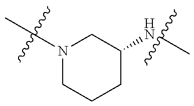 | 404.1 |
| 11 | 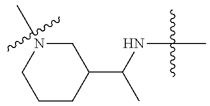 | 432.1 |
| 12 | 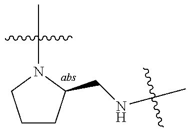 [SMILES: *NC[C@H]1CCCN1*] | 404.1 |
| 13 | 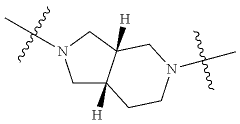 [SMILES: *N1CC[C@@H]2CN(*)C[C@@H]2C1] | 430.0 |
| 14 | 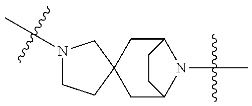 [SMILES: *N1CCC2(CC3CCC(C2)N3*)C1] | 470.2 |
| 15 | 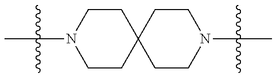 [SMILES: *N1CCC2(CC1)CCN(*)CC2] | 458.1 |
| 16 | 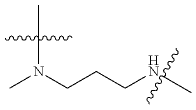 | 392.1 |
| 17 | 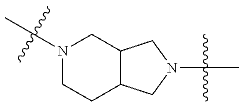 [SMILES: *N1CCC2CN(*)CC2C1] | 430.1 |
| 18 | 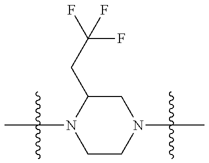 [SMILES: *N1CCN(*)C(CC(F)(F)F)C1] | 470.0 |
| 19 | 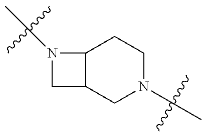 [SMILES: *N1CCC2C(C1)CN2*] | 416.1 |
| 20 | 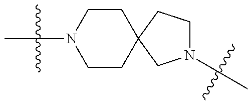 [SMILES: *N1CCC2(CC1)CCN(*)C2] | 444.1 |
| 21 | 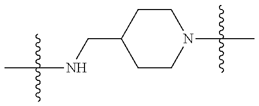 [SMILES: *NCC1CCN(*)CC1] | 418.1 |
| 22 | 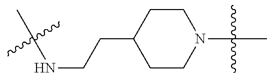 [SMILES: *NCCC1CCN(*)CC1] | 432.0 |
| 23 | 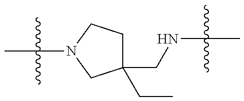 [SMILES: *NCC1(CC)CCN(*)C1] | 432.1 |
| 24 | 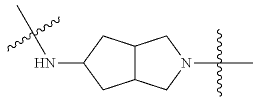 [SMILES: *NC1CC2CN(*)CC2C1] | 430.0 |
| 25 | 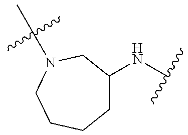 [SMILES: *NC1CCCCN(*)C1] | 418.1 |
| 26 | 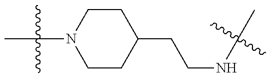 [SMILES: *NCCC1CCN(*)CC1] | 432.0 |
| 27 | 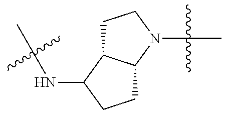 | 430.1 |
| 28 | 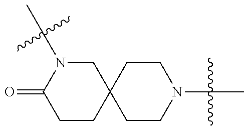 [SMILES: *N1CCC2(CCC(=O)N(*)C2)CC1] | 472.1 |
| 29 | 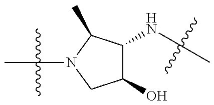 [SMILES: *N[C@@H]1[C@@H](O)CN(*)[C@H]1C] | 420.1 |
| 30 | 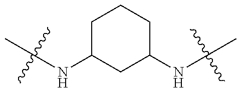 [SMILES: *NC1CCCC(N*)C1] | 418.1 |
| 31 | 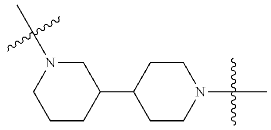 [SMILES: *N1CCC(C2CCCN(*)C2)CC1] | 472.1 |
| 32 | 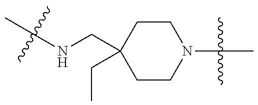 [SMILES: *NCC1(CC)CCN(*)CC1] | 446.1 |
| 33 | 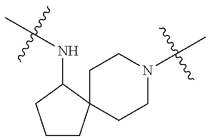 [SMILES: *NC1CCCC12CCN(*)CC2] | 458.1 |
| 34 | 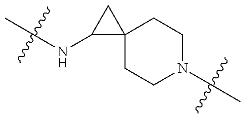 [SMILES: *NC1CC12CCN(*)CC2] | 430.1 |
| 35 | 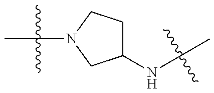 [SMILES: *NC1CCN(*)C1] | 390.1 |
| 36 | 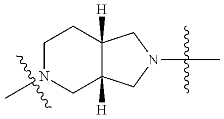 | 430.1 |
| 37 | 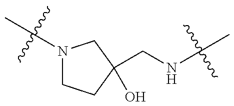 [SMILES: *NCC1(O)CCN(*)C1] | 420.1 |
| 38 | 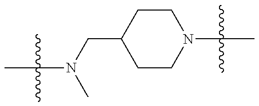 [SMILES: *N(C)CC1CCN(*)CC1] | 432.1 |
| 39 | 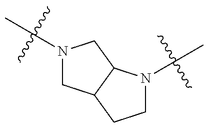 [SMILES: *N1CC2CCN(*)C2C1] | 416.1 |
| 40 | 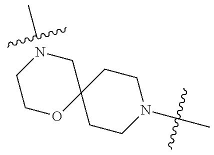 | 460.0 |
| 41 | 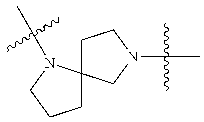 [SMILES: *N1CCC2(CCCN2*)C1] | 430.1 |
| 42 | 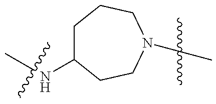 | 418.1 |
| 43 | 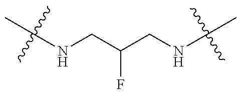 [SMILES: *NCC(F)CN*] | 396.0 |
| 44 | 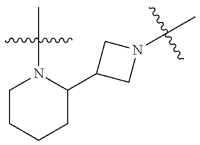 [SMILES: *N1CC(C2CCCCN2*)C1] | 444.0 |
| 45 | 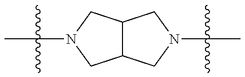 [SMILES: *N1CC2CN(*)CC2C1] | 416.1 |
| 46 | 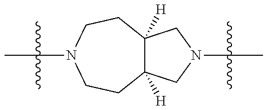 [SMILES: *N1CC[C@@H]2CN(*)C[C@@H]2CC1] | 444.2 |
| 47 | 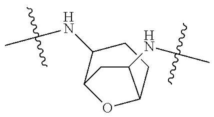 [SMILES: *NC1CC2OC1CCC2N*] | 446.1 |
| 48 | 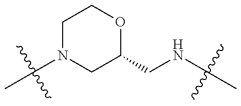 [SMILES: *NC[C@@H]1CN(*)CCO1] | 420.0 |
| 49 | 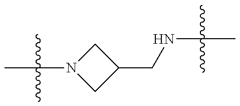 [SMILES: *NCC1CN(*)C1] | 390.1 |
| 50 | 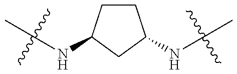 [SMILES: *N[C@H]1CC[C@H](N*)C1] | 404.1 |
| 51 | 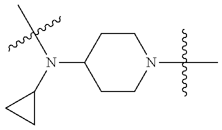 [SMILES: *N1CCC(N(*)C2CC2)CC1] | 444.0 |
| 52 | 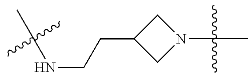 [SMILES: *NCCC1CN(*)C1] | 404.1 |
| 53 | 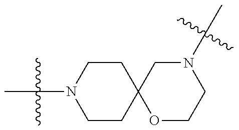 [SMILES: *N1CCC2(CC1)CN(*)CCO2] | 460.1 |
| 54 | 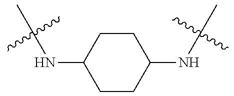 [SMILES: *NC1CCC(N*)CC1] | 418.1 |
| 55 | 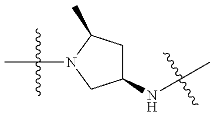 [SMILES: *N[C@@H]1C[C@H](C)N(*)C1] | 404.1 |
| 56 | 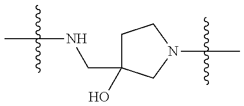 [SMILES: *NCC1(O)CCN(*)C1] | 420.1 |
| 57 | 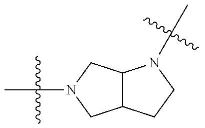 [SMILES: *N1CC2CCN(*)C2C1] | 416.1 |
| 58 | 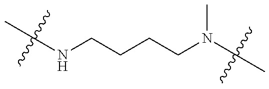 [SMILES: *NCCCCN(*)C] | 406.1 |
| 59 | 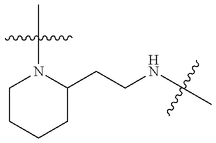 [SMILES: *NCCC1CCCCN1*] | 432.2 |
| 60 | 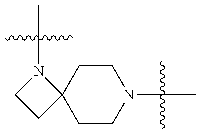 [SMILES: *N1CCC2(CC1)CCN2*] | 458.2 |
| 61 | 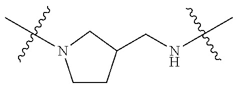 [SMILES: *NCC1CCN(*)C1] | 404.1 |
| 62 | 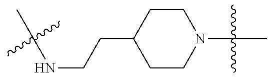 [SMILES: *NCCC1CCN(*)CC1] | 432.0 |
| 63 | 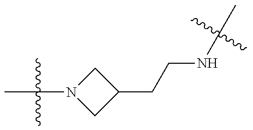 [SMILES: *NCCC1CN(*)C1] | 404.1 |
| 64 | 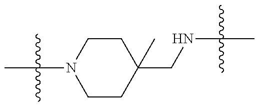 [SMILES: *NCC1(C)CCN(*)CC1] | 432.1 |
| 65 | 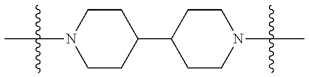 [SMILES: *N1CCC(C2CCN(*)CC2)CC1] | 472.1 |
| 66 | 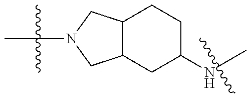 | 444.1 |
| 67 | 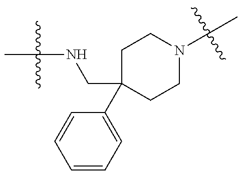 [SMILES: *NCC1(c2ccccc2)CCN(*)CC1] | 494.0 |
| 68 | 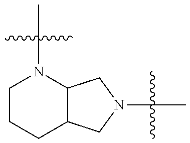 [SMILES: *N1CC2CCCN(*)C2C1] | 430.1 |
| 69 | 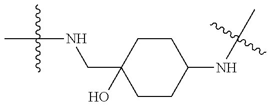 [SMILES: *NCC1(O)CCC(N*)CC1] | 448.1 |
| 70 | 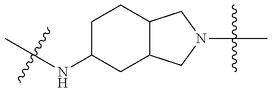 [SMILES: *NC1CCC2CN(*)CC2C1] | 444.1 |
| 71 | 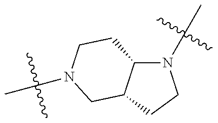 [SMILES: *N1CC[C@H]2[C@H](CCN2*)C1] | 430.1 |
| 72 | 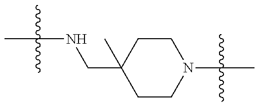 [SMILES: *NCC1(C)CCN(*)CC1] | 432.1 |
| 73 | 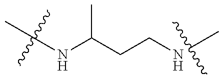 | 392.1 |
| 74 | 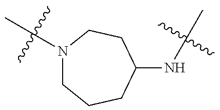 [SMILES: *NC1CCCN(*)CC1] | 418.1 |
| 75 | 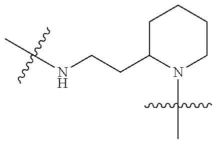 [SMILES: *NCCC1CCCCN1*] | 432.2 |
| 76 | 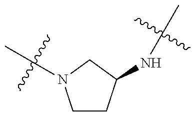 [SMILES: *N[C@H]1CCN(*)C1] | 390.1 |
| 77 | 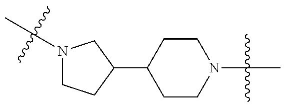 [SMILES: *N1CCC(C2CCN(*)C2)CC1] | 458.2 |
| 78 | 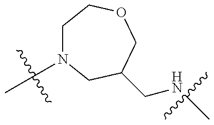 | 434.1 |
| 79 | 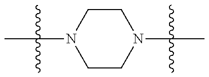 [SMILES: *N1CCN(*)CC1] | 390.1 |
| 80 | 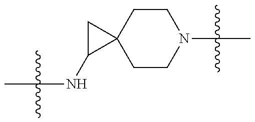 [SMILES: *NC1CC12CCN(*)CC2] | 430.1 |
| 81 | 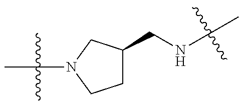 [SMILES: *NC[C@H]1CCN(*)C1] | 404.1 |
| 82 | 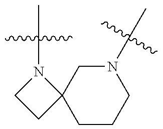 [SMILES: *N1CCCC2(CCN2*)C1] | 430.1 |

The following additional compounds were prepared according to the method described in Example 1, step 1, and where appropriate, step 2 and step 3 as well.

| No. | | [M + H]+ (Obs'd) |
|---|---|---|
| 83 | 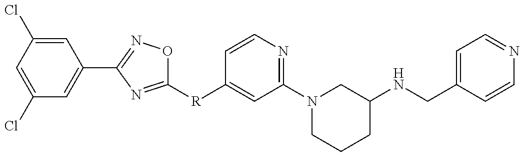 | 404.1 |
| 84 | 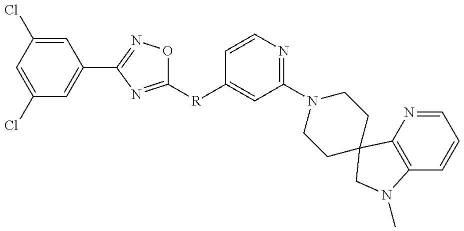 | 416.1 |

### Example 2

### Preparation of Compound 85—5-(3,5-dimethoxyphenyl)-3-(9-(2-methylpyridin-4-yl)-2,9-diazaspiro[5.5]undecan-2-yl)-1,2,4-oxadiazole

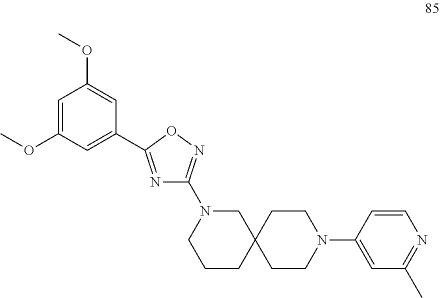

3-bromo-5-(3,5-dimethoxyphenyl)-1,2,4-oxadiazole (54.9 mg, 0.193 mmol) was added to a mixture of 9-(2-methylpyridin-4-yl)-2,9-diazaspiro[5.5]undecane (125 mg, 0.193 mmol) (37.8% content), and K₂CO₃ (266 mg, 1.926 mmol) in DMF (2 mL) at 22° C. After the addition completed, the suspension was allowed to stir at 22° C. for 15 hours. The reaction mixture was cooled to room temperature, diluted with MeCN (3 mL), the mixture was purified using prep-HPLC after filtration (Column: Waters Xbridge Prep OBD C18 100×19 mm×5 um. Condition: water (0.1% TFA v/v)-ACN, begin B 26%, end B 56%, gradient time (min): 10, 100% B hold time (min): 2. Flow Rate (ml/min): 25.), and lyophilized to provide 5-(3,5-dimethoxyphenyl)-3-(9-(2-methylpyridin-4-yl)-2,9-diazaspiro[5.5]undecan-2-yl)-1,2,4-oxadiazole. MS ESI calc'd. for C₂₅H₃₂N₅O₃ [M+H]⁺, 450.2, found, 450.2. ¹H NMR (400 MHz, CD₃OD): δ 7.95 (d, J=7.04 Hz, 1H), 7.16 (d, J=2.4 Hz, 2H), 6.95-7.00 (m, 2H), 6.73 (t, J=2.4 Hz, 1H), 3.84 (s, 6H), 3.74-3.81 (m, 2H), 3.62-3.70 (m, 2H), 3.52-3.54 (t, J=5.5 Hz, 2H), 3.49 (s, 2H), 2.49 (s, 3H), 1.74-1.80 (m, 4H), 1.58-1.67 (m, 4H).

The following compounds were prepared according to the method described in Example 2.

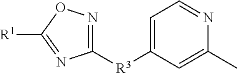

| No. | R¹ | R³ | [M + H]+ Obs'd | ¹H NMR |
|---|---|---|---|---|
| 86 | 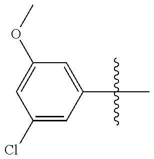 [SMILES: *c1cc(Cl)cc(OC)c1] | 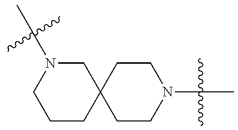 [SMILES: *N1CCC2(CCCN(*)C2)CC1] | 454.1. | ¹H NMR (400 MHz, CD₃OD) δ 7.95 (d, J = 7.2 Hz, 1H), 7.61 (t, J = 1.5 Hz, 1H), 7.49 (dd, J = 2.4, 1.4 Hz, 1H), 7.23 (t, J = 2.1 Hz, 1H), 6.97-7.01 (m, 2H), 3.88 (s, 3H), 3.75-3.83 (m, 2H), 3.63-3.71 (m, 2H) 3.54 (t, J = 5.5 Hz, 2H), 3.50 (s, 2H), 2.50 (s, 3H), 1.72- 1.80 (m, 4H), 1.58-1.68 (m, 4 H). |
| 87 | 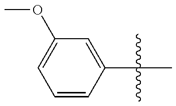 [SMILES: *c1cccc(OC)c1] | 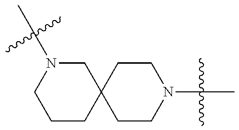 [SMILES: *N1CCC2(CCCN(*)C2)CC1] | 420.2 | ¹H NMR (400 MHz, CD₃OD) δ 7.95 (d, J = 7.0 Hz, 1H), 7.61- 7.63 (m, 1H), 7.55 (dd, J = 2.5, 1.37 Hz, 1H), 7.46 (t, J = 8.0 Hz, 1H), 7.17-7.21 (m, 1H), 6.96-7.00 (m, 2H), 3.87 (s, 3H), 3.75-3.82 (m, 2H), 3.63-3.70 (m, 2H), 3.54 (t, J = 5.5 Hz, 2H), 3.50 (s, 2H), |
| | | | | 2.50 (s, 3H), 1.74-1.80 (m, 4H), |
| | | | | 1.58-1.67 (m, 4 H). |
| 88 | 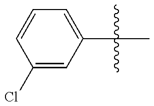 [SMILES: *c1cccc(Cl)c1] | 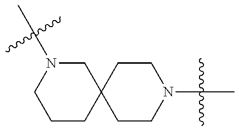 [SMILES: *N1CCC2(CCCN(*)C2)CC1] | 424.1 | ¹H NMR (400 MHz, CD₃OD) δ 8.04 (d, J = 16 Hz, 1H), 7.96 (dd, J = 13.7, 7.4 Hz, 2H), 7.62-7.67 (m, 1H), 7.53-7.58 (m, 1H), 6.96- 7.00 (m, 2H), 3.75-3.82 (m, 2H), 3.62-3.70 (m, 2H) 3.54 (t, J = 5.5 Hz, 2H), 3.51 (s, 2H), 2.49 (s, 3H) 1.72-1.80 (m, 4H), 1.57-1.68 (m, |
| | | | | 4 H). |
| 89 | 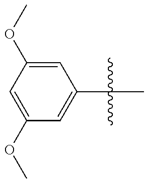 [SMILES: *c1cc(OC)cc(OC)c1] | 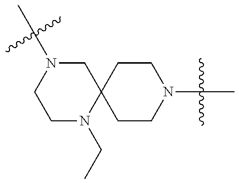 [SMILES: *N1CCC2(CC1)CN(*)CCN2CC] | 479.4 | ¹H NMR (400 MHz, CD₃OD) δ 8.06 (d, J = 6.8 Hz, 1H), 7.20 (d, J = 2.4 Hz, 2H), 7.09-7.03 (m, 2H), 6.78 (t, J = 2.2 Hz, 1H), 4.25-4.16 (m, 2H), 3.95-3.67 (m, 8H), 3.66- 3.48 (m, 6H), 3.34-3.33 (m, 2H), 2.60-2.50 (m, 3H), 2.44-2.08 (m, 4H), 1.43 (t, J = 7.2 Hz, 3H). |
| 90 | 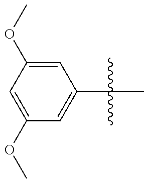 [SMILES: *c1cc(OC)cc(OC)c1] | 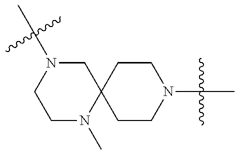 [SMILES: *N1CCC2(CC1)CN(*)CCN2C] | 465.3 | ¹H NMR (400 MHz, CD₃OD) δ 8.06 (d, J = 7.3 Hz, 1H), 7.20 (d, J = 2.2 Hz, 2H), 7.10-7.03 (m, 2H), 6.77 (t, J= 2.3 Hz, 1H), 4.23-4.13 (m, 2H), 4.09-3.81 (m, 10H), 3.68-3.52 (m, 4H), 3.00 (s, 3H), 2.54 (s, 3H), 2.30-2.20 (m, 4H). |

### Example 3

### Preparation of Compound 91—5-(3-methoxy-5-(trifluoromethoxy)phenyl)-3-(9-(2-methylpyridin-4-yl)-2,9-diazaspiro[5.5]undecan-2-yl)-1,2,4-oxadiazole

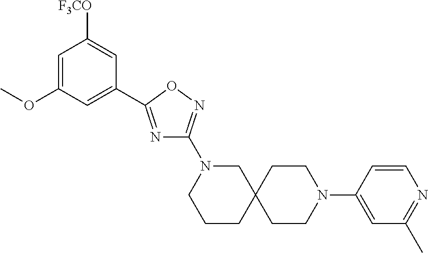

### Step 1:

PdCl₂(dppf) (54.0 mg, 0.074 mmol) was added to a mixture of 1-bromo-3-methoxy-5-(trifluoromethoxy)benzene (200 mg, 0.738 mmol), and sodium acetate (121 mg, 1.476 mmol) in MeOH (10 mL) at 17° C. After the addition was complete, the mixture was allowed to stir under an atmosphere of CO (50 psi) at 70° C. for 15 hours. After cooling to room temperature, the mixture was concentrated in vacuo to provide a residue, which was diluted with EtOAc (10 mL). The organic layer was washed with H₂O (10 mL). The water layer was extracted with EtOAc (10 mL×2). The collected organic layers were washed with brine (10 mL), and dried over anhydrous Na₂SO₄. The reaction mixture was filtered, and the filtrate was concentrated in vacuo to provide a residue as a red liquid. The residue was purified using prep-TLC (SiO₂, petroleum ether:EtOAc=10:1) to provide methyl 3-methoxy-5-(trifluoromethoxy)benzoate. ¹H NMR (400 MHz, CDCl₃): δ 7.52 (dd, J=2.3, 1.3 Hz, 1H), 7.48-7.51 (m, 1H), 6.96 (s, 1H), 3.94 (s, 3H), 3.87 (s, 3H).

### Step 2:

NaH (6.33 mg, 0.158 mmol) (60% in mineral oil) was added to a mixture of (E)-N′-hydroxy-9-(2-methylpyridin-4-yl)-2,9-diazaspiro[5.5]undecane-2-carboximidamide (40 mg, 0.132 mmol) in THF (2 mL) at 17° C. (room temperature) under N₂ protection. After the addition was completed, the mixture was allowed to stir at 17° C. for 30 minutes. Methyl 3-methoxy-5-(trifluoromethoxy)benzoate (49.5 mg, 0.198 mmol) was added to this mixture, and the resulting mixture was heated to 70° C., and stirred for 1.5 hours under N₂. After cooling to room temperature, the mixture was concentrated in vacuo to provide a residue, which was diluted with EtOAc (10 mL). The organic layer was washed with H₂O (10 mL). The water layer was extracted with EtOAc (10 mL×2). The collected organic layers were washed with brine (10 mL), and dried over anhydrous Na₂SO₄. The reaction mixture was filtered, and the filtrate was concentrated in vacuo to provide a residue as a liquid. The residue was purified using prep-HPLC (Column: Waters XSELECT C18 150×30 mm×5 um. Condition: water (0.1% TFA v/v)-ACN, begin B 32%, end B 62%, gradient time (min): 10, 100% B hold time (min) 2. Flow Rate (ml/min): 25.), and lyophilized to provide 5-(3-methoxy-5-(trifluoromethoxy)phenyl)-3-(9-(2-methylpyridin-4-yl)-2,9-diazaspiro[5.5]undecan-2-yl)-1,2,4-oxadiazole. MS ESI calc'd. for C₂₅H₂₉F₃N₅O₃ [M+H]⁺ 504.2, found 504.1. ¹H NMR (400 MHz, CD₃OD): δ 7.96 (d, J=7.0 Hz, 1H), 7.56 (s, 1H), 7.49 (s, 1H), 7.11 (s, 1H), 6.98-7.00 (m, 2H), 3.91 (s, 3H), 3.75-3.82 (m, 2H), 3.63-3.70 (m, 2H), 3.54 (t, J=5.5 Hz, 2H), 3.50 (s, 2H), 2.50 (s, 3H), 1.74-1.80 (m, 4H), 1.59-1.68 (m, 4H).

The following compound was prepared according to the method described in Example 3.

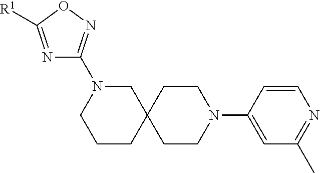

| No. | R¹ | [M + H]+ Obs'd | ¹H NMR |
|---|---|---|---|
| 92 | 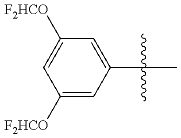 [SMILES: *c1cc(OC(F)F)cc(OC(F)F)c1] | 522.1 | ¹H NMR (400 MHz, CD₃OD) δ 7.96 (d, J = 7.0 Hz, 1H), 7.67 (d, J = 1.8 Hz, 2H), 7.23 (t, 1H), 6.97-7.00 (m, 2H), 6.83-7.20 (t, 2H), 3.76-3.82 (m, 2H), 3.64-3.70 (m, 2H), 3.55 (t, J = 5.3 Hz, 2H), 3.51 (s, 2H), 2.50 (s, 3H), 1.75 (m, 4H), 1.59-1.68 (m, 4H). |

### Example 4

### Preparation of Compound 93—3-(3,5-dimethoxyphenyl)-5-(9-(2-methylpyridin-4-yl)-1-(2,2,2-trifluoroethyl)-1,4,9-triazaspiro[5.5]undecan-4-yl)-1,2,4-oxadiazole

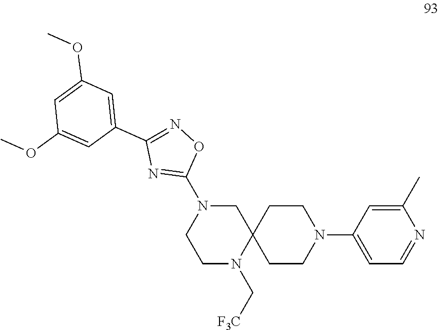

Et₃N (0.062 mL, 0.442 mmol) was added to a stirred mixture of 9-(2-methylpyridin-4-yl)-1-(2,2,2-trifluoroethyl)-1,4,9-triazaspiro[5.5]undecane (29 mg, 0.088 mmol), and 5-chloro-3-(3,5-dimethoxyphenyl)-1,2,4-oxadiazole (21.25 mg, 0.088 mmol) in DMF (I mL), and the reaction was allowed to stir at room temperature around 15° C. for 30 minutes. The reaction was purified using prep-HPLC(Column Waters XSELECT C18 150×30 mm×5 um. Condition: water (0.1% TFA)-ACN, begin B 32%, end B 52%, gradient time (min): 10. 100% B hold time (min): 2. Flow Rate (ml/min): 25.) to provide 3-(3,5-dimethoxyphenyl)-5-(9-(2-methylpyridin-4-yl)-1-(2,2,2-trifluoroethyl)-1,4,9-triazaspiro[5.5]undecan-4-yl)-1,2,4-oxadiazole. MS ESI calc'd for C₂₆H₃₁F₃N₆O₃ [M+H]⁺ 533.4, found 533.4. ¹H NMR (400 MHz, CD₃OD) δ 7.99 (d, J=7.1 Hz, 1H), 7.08 (d, J=2.0 Hz, 2H), 7.04-6.98 (m, 2H), 6.63 (s, 1H), 4.07 (d, J=13.9 Hz, 2H), 3.84-3.80 (m, 8H), 3.77 (t, J=4.4 Hz, 2H), 3.56-3.51 (m, 2H), 3.24 (q, J=9.4 Hz, 2H), 3.08-3.03 (m, 2H), 2.51 (s, 3H), 1.90-1.89 (m, 41H).

The following compounds were prepared according to the method described in Example 4.

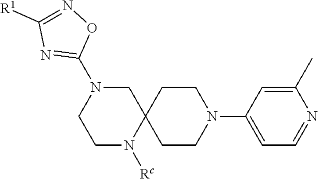

| No. | R¹ | R^{c} | [M + H]+ Obs'd | HNMR |
|---|---|---|---|---|
| 94 | 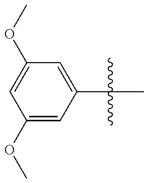 [SMILES: *c1cc(OC)cc(OC)c1] | 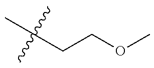 [SMILES: *CCOC] | 509.3 | ¹H NMR (400 MHz, CD₃OD) δ 8.05 (d, J = 7.2 Hz, 1H), 7.10-7.04 (m, 4H), 6.64 (t, J = 2.4 Hz, 1H), 4.17- 4.14 (m, 4H), 4.03-4.00 (m, 2H), 3.82 (s, 6H), 3.74-3.72 (m, 2H), 3.64-3.62 (m, 4H), 3.46-3.44 (m, 2 H), 3.41 (s, 3H), 2.53 (s, 3H), 2.28- 2.15 (m, 4H). |
| 95 | 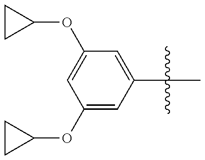 [SMILES: *c1cc(OC2CC2)cc(OC2CC2)c1] | 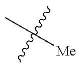 | 517.1 | ¹H NMR (400 MHz, CD₃OD) δ 8.06 (d, J = 7.5 Hz, 1H), 7.25 (d, J = 2.2 Hz, 2H), 7.08-7.04 (m, 2H), 6.91 (t, J = 2.4 Hz, 1H), 4.24-4.11 (m, 4H), 4.10-3.99 (m, 2H), 3.81 (tt, J = 3.1, 5.9 Hz, 2H), 3.63-3.48 (m, 4H), 2.96 (s, 3H), 2.54 (s, 3H), 2.29-2.15 (m, 4H), 0.84-0.70 (m, 8H). |
| 96 | 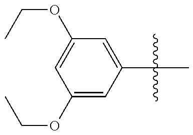 [SMILES: *c1cc(OCC)cc(OCC)c1] | 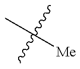 | 493.1 | ¹H NMR (400 MHz, CD₃OD) δ 8.05 (d, J = 6.8 Hz, 1H), 7.13-6.99 (m, 4H), 6.60 (t, J = 2.1 Hz, 1H), 4.27-4.12 (m, 4H), 4.04 (q, J = 7.0 Hz, 6H), 3.66-3.49 (m, 4H), 2.99 (s, 3H), 2.54 (s, 3H), 2.31-2.14 (m, 4H), 1.38 (t, J = 7.1 Hz, 6H). |
| 97 | 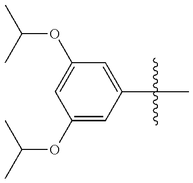 [SMILES: *c1cc(OC(C)C)cc(OC(C)C)c1] | 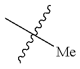 | 521.4 | ¹H NMR (400 MHz, CD₃OD) δ 8.06 (d, J = 6.8 Hz, 1H), 7.09-7.03 (m, 4H), 6.58 (t, J = 2.2 Hz, 1H), 4.61 (t, J = 6.0 Hz, 2H), 4.27-4.12 (m, 4H), 4.12-4.00 (m, 2H), 3.68-3.49 (m, 4H), 2.99 (s, 3H), 2.54 (s, 3H), 2.31-2.15 (m, 4H), 1.31 (d, J = 6.0 Hz, 12H). |
| 98 | 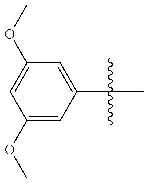 [SMILES: *c1cc(OC)cc(OC)c1] | 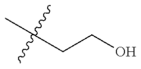 [SMILES: *CCO] | 495.1 | ¹H NMR (400 MHz, CD₃OD) δ 8.05 (d, J = 7.2 Hz, 1H), 7.10-7.04 (m, 4H), 6.64 (t, J = 2.4 Hz, 1H), 4.17- 4.14 (m, 4H), 4.03-4.00 (m, 2H), 3.89-3.87 (m, 2H), 3.82 (s, 6H), 3.64-3.62 (m, 4H), 3.46-3.44 (m, 2 H), 2.53 (s, 3H), 2.28-2.15 (m, 4H). |
| 99 | 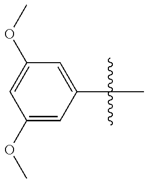 [SMILES: *c1cc(OC)cc(OC)c1] | 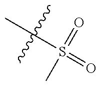 [SMILES: *S(C)(=O)=O] | 529.2 | ¹H NMR (400 MHz, CD₃OD) δ 8.00 (d, J = 6.6 Hz, 1H), 7.09 (d, J = 2.2 Hz, 2H), 6.76-6.69 (m, 2H), 6.62 (t, J = 2.2 Hz, 1H), 4.07 (s, 2H), 3.93 (d, J = 13.6 Hz, 2H), 3.82-3.80 (m, 9H), 3.24 (t, J = 11.4 Hz, 2H), 3.06 (s, 3H), 2.67-2.57 (m, 2H), 2.40 (s, 3H), 2.10-2.03 (m, 2H). |
| 100 | 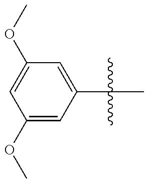 [SMILES: *c1cc(OC)cc(OC)c1] | 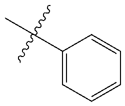 [SMILES: *c1ccccc1] | 527.3 | ¹H NMR (400 MHz, CD₃OD) δ 7.89 (d, J = 7.3 Hz, 1H), 7.34-7.29 (m, 2H), 7.27-7.22 (m, 2H), 7.21-7.16 (m, 1H), 7.11 (d, J = 2.2 Hz, 2H), 6.89-6.83 (m, 2H), 6.64 (t, J = 2.2 Hz, 1H), 3.99 (s, 2H), 3.88-3.80 (m, 10H), 3.50-3.35 (m, 4H), 2.45 (s, 3H), 2.07-2.03 (m, 2H), 1.91-1.80 (m, 2H). |
| 101 | 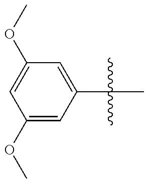 [SMILES: *c1cc(OC)cc(OC)c1] | 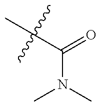 [SMILES: *C(=O)N(C)C] | 522.3 | ¹H NMR (400 MHz, CD₃OD) δ 8.28 (d, J = 8.0 Hz, 1H), 7.27-7.18 (m, 2H), 7.11 (d, J = 2.0 Hz, 2H), 6.66- 6.63 (m, 1H), 4.20 (d, J = 14.1 Hz, 2H), 4.11 (s, 2H), 4.01 (s, 2H), 3.82 (s, 6H), 3.78-3.69 (m, 2H), 3.53- 3.47 (m, 2H), 3.20 (s, 3H), 2.88 (s, 3H), 2.54 (s, 3H), 2.30 (d, J = 14.1 Hz, 2H), 12.08 (s, 2H). |

The following compounds were prepared according to the method described in Example 4.

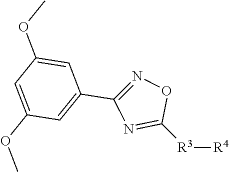

| No. | —R³—R⁴ | [M + H]+ Obs'd | HNMR |
|---|---|---|---|
| 102 | 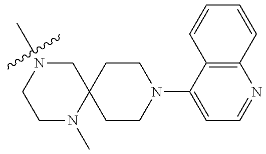 | 501.4 | ¹H NMR (400 MHz, CD₃OD) δ 8.58 (d, J = 6.8 Hz, 1H), 8.30 (d, J = 8.6 Hz, 1H), 8.03- 7.94 (m, 2H), 7.70-7.73 (m, 1H), 7.25 (d, J = 7.1 Hz, 1H), 7.11 (d, J = 2.2 Hz, 2H), 6.64 (t, J = 2.3 Hz, 1H), 4.33-4.21 (m, 4H), 4.13- 4.05 (m, 2H), 3.94-3.85 (m, 2H), 3.82 (s, 3H), 3.31 (td, J = 1.6, 3.4 Hz, 3H), 3.09 (s, 3H), 2.55-2.40 (m, 2H), 2.31-2.27 (m, 2H). |
| 103 | 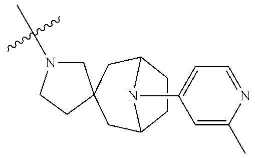 [SMILES: *N1CCC2(CC3CCC(C2)N3c2ccnc(C)c2)C1] | 462.3 | ¹H NMR (400 MHz, CD₃OD) δ 7.97 (d, J = 7.6 Hz, 1H), 7.07 (d, J = 2.2 Hz, 2H), 6.94- 6.88 (m, 2H), 6.61 (t, J = 2.2 Hz, 1H), 4.67 (s, 2H), 3.84-3.81 (m, 8H), 3.28 (s, 2H), 2.50 (s, 3H), 2.36 (t, J = 7.0 Hz, 2H), 2.20- 2.14 (m, 2H), 2.04-1.87 (m, 4H). |

### Example 5

### Preparation of Compound 104—N-((1-((3,5-dichlorobenzyl)sulfonyl)piperidin-3-yl)methyl)-N,2-dimethylpyridin-4-amine

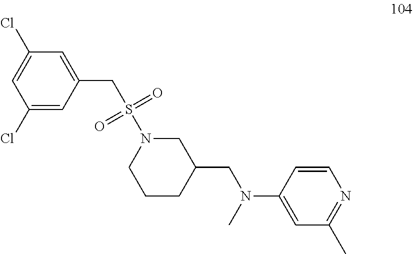 [SMILES: Cc1cc(N(C)CC2CCCN(S(=O)(=O)Cc3cc(Cl)cc(Cl)c3)C2)ccn1]

### Step 1:

The solution of tert-butyl 3-((methylamino)methyl)piperidine-1-carboxylate (100 mg, 0.438 mmol), 4-chloro-2-methylpyridine (61.5 mg, 0.482 mmol), and DIPEA (0.153 mL, 0.876 mmol) in NMP (1 mL) was allowed to stir for 2 hours at 160° C. The reaction mixture was poured in water (50 mL), and saturated brine (5 mL) then extracted with EtOAc (10 mL×3). The organic layers were dried over Na₂SO₄, filtered, and the filtrate was concentrated. The residue was purified using prep-TLC on silica gel (DCM:MeOH=5:1) to provide tert-butyl 3-((methyl(2-methylpyridin-4-yl)amino)methyl)piperidine-1-carboxylate. Additional purification through preparative HPLC on fitted with a Waters XSELECT C18 150×30 mm×5 um using water (0. % TFA)-ACN as the eluents. Mobile phase A: 0.1% TFA. Mobile phase B: ACN. Gradient: 17-47% B, 0-10.0 min; 100% B, 10.1-12.0 min; 10% B, 12.1-15 minutes. Flow rate: 25 mL/min.

### Step 2:

A solution of tert-butyl 3-((methyl(2-methylpyridin-4-yl)amino)methyl)piperidine-1-carboxylate (120 mg, 0.376 mmol), and 4 M HCl-dioxane (0.5 mL) in DCM (2 mL) was allowed to stir for 1 hour at 10° C. The reaction mixture was concentrated in vacuo to provide N,2-dimethyl-N-(piperidin-3-ylmethyl)pyridin-4-amine. MS ESI calc'd. for C₁₃H₂₂N₃ [M+H]⁺, 220.2, found, 220.2.

### Step 3:

A solution of N,2-dimethyl-N-(piperidin-3-ylmethyl)pyridin-4-amine (30 mg, 0.137 mmol), (3,5-dichlorophenyl)methanesulfonyl chloride (71.0 mg, 0.274 mmol), and DIPEA (0.072 mL, 0.410 mmol) in DMF (2 mL) was allowed to stir at 40° C. for 1 hour. The desired mass was detected by LCMS. The residue was purified using acidic prep-HPLC (TFA) to provide the title compound N-((1-((3,5-dichlorobenzyl)sulfonyl)piperidin-3-yl)methyl)-N,2-dimethylpyridin-4-amine. Additional purification through preparative HPLC on fitted with a Waters XSELECT C18 150×30 mm×5 um using water (0.1% TFA)-ACN as the eluents. Mobile phase A: 0.1% TFA. Mobile phase B: ACN. Gradient: 20-40% B, 0-10.0 min; 100% B, 10.1-12.0 min; 10% B, 12.1-15 minutes. Flow rate: 25 mL/min. MS (ESI) calcd. for C₂₀H₂₆Cl₂N₃O₂S [M+H]⁺,442.1, found, 442.0. ¹H NMR (400 MHz, CD₃OD) δ 7.98 (s, 1H), 7.40 (s, 3H), 6.88-6.87 (m, 2H), 4.32 (s, 2H), 3.54-3.46 (m, 3H), 3.17 (s, 3H), 3.00-2.98 (m, 1H), 2.69-2.67 (m, 1H), 2.53 (s, 3H), 2.01-1.90 (m, 1H), 1.80-1.78 (m, 2H), 1.57-1.55 (m, 1H), 1.35-1.28 (m, 2H).

### Example 6

### Preparation of Compound 105—6-(2-(3,5-dichlorophenyl)-2H-1,2,3-triazol-4-yl)-2-(2-methylpyridin-4-yl)-2,6-diazaspiro[3.5]nonane

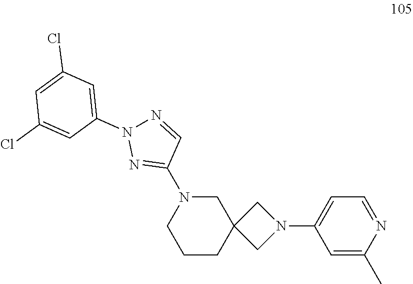 [SMILES: Cc1cc(N2CC3(CCCN(c4cnn(-c5cc(Cl)cc(Cl)c5)n4)C3)C2)ccn1]

### Step 1:

To a suspension of 2-(3,5-dichlorophenyl)-2H-1,2,3-triazole 1-oxide (100 mg, 0.435 mmol), copper (II) acetate (15.79 mg, 0.087 mmol), and potassium phosphate tribasic (185 mg, 0.869 mmol) in DME (3 mL) was added tert-butyl 2,6-diazaspiro[3.5]nonane-2-carboxylate (295 mg, 1.304 mmol) under an air atmosphere, and the reaction was allowed to stir at 80° C. for 24 hours. The reaction mixture was cooled to room temperature. The reaction mixture was diluted with H₂O (3 mL), and extracted with EtOAc (5 mL×3). The combined organic layers were washed with brine (5 mL), dried over Na₂SO₄ and filtered. The filtrate was concentrated by rotary evaporator. The residue was purified using Prep-HPLC (Column: Waters XSELECT C18 150×30 mm×5 um, condition water (0.1% TFA)-ACN, begin B 54%, End B 84%, gradient time (min): 10, 100% B hold time (min): 2, flow rate (mL/min): 25.) to provide tert-butyl 6-(2-(3,5-dichlorophenyl)-2H-1,2,3-triazol-4-yl)-2,6-diazaspiro[3.5]nonane-2-carboxylate. ¹H NMR (400 MHz, CDCl₃) δ 7.96-8.01 (m, 2H), 7.43-7.46 (m, 1H), 7.40-7.43 (m, 1H), 3.76-3.85 (m, 2H), 3.62-3.69 (m, 2H), 3.33-3.47 (m, 2H), 3.16-3.27 (m, 2H), 1.70-1.86 (m, 4H), 1.45 (s, 9H).

### Step 2:

A solution of tert-butyl 6-(2-(3,5-dichlorophenyl)-2H-1,2,3-triazol-4-yl)-2,6-diazaspiro[3.5]nonane-2-carboxylate (30 mg, 0.068 mmol) in a HCl solution in dioxane (17.11 μl, 0.068 mmol, 4M) was allowed to stir at 0° C. for 30 minutes. The solvent was removed by rotary evaporator. The residue was dissolved in 5 mL of EtOAc and washed with sat, NaHCO₃ (5 mL), and brine (5 mL). The organic layer was dried over MgSO₄ and filtered. The filtrate was concentrated by rotary evaporator to provide 6-(2-(3,5-dichlorophenyl)-2H-1,2,3-triazol-4-yl)-2,6-diazaspiro[3.5]nonane as a yellow oil which was used without further purification. MS ESI calc'd. for C₃₁H₃₉Cl₄N₁₀O [2M+MeOH+H]⁺, 709.2, found, 709.2.

### Step 3:

A solution of 6-(2-(3,5-dichlorophenyl)-2H-1,2,3-triazol-4-yl)-2,6-diazaspiro[3.5]-nonane (20 mg, 0.059 mmol), 4-chloro-2-methylpyridine (0.013 mL, 0.118 mmol), and DIPEA (0.052 mL, 0.296 mmol) in NMP (0.2 mL) was sealed in a microwave tube. The reaction was heated by microwave to 120° C. for 30 minutes. LCMS showed the reaction was completed. The reaction mixture was cooled to room temperature and filtered. The filtrate was purified using prep-HPLC (Column: Waters XSELECT C18 150×30 mm×5 um, condition: water (0.1% TFA)-ACN, begin B 38%, end B 68%, gradient time (min): 10, 100% B hold time (min): 2, flow rate (mL/min): 25.) to provide 6-(2-(3,5-dichlorophenyl)-2H-1,2,3-triazol-4-yl)-2-(2-methylpyridin-4-yl)-2,6-diazaspiro[3.5]nonane. MS ESI calc'd. for C₂₃H₂₃Cl₂N₆ [M+H]⁺, 429.1, found, 429.0. ¹H NMR (400 MHz, CDCl₃) & 8.18-8.25 (m, 1H), 7.83-7.89 (m, 2H), 7.32 (s, 1H), 7.22-7.25 (m, 1H), 6.27-6.33 (m, 1H), 6.16 (s, 1H), 3.96-4.04 (m, 2H), 3.86-3.94 (m, 2H), 3.48-3.55 (m, 2H), 3.22-3.31 (m, 2H), 2.60 (s, 3H), 1.91-1.98 (m, 2H), 1.26 (s, 2H).

### Example 7

### Preparation of Compound 106—5-(5-(1-methyl-9-(2-methylpyridin-4-yl)-1,4,9-triazaspiro[5.5]undecan-4-yl)-1,2,4-oxadiazol-3-yl)benzene-1,3-diol

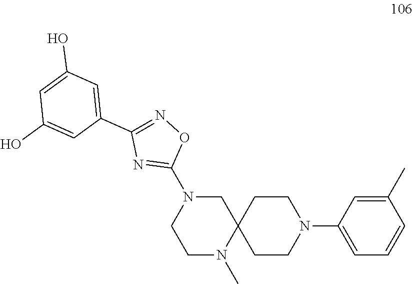 [SMILES: Cc1cccc(N2CCC3(CC2)CN(c2nc(-c4cc(O)cc(O)c4)no2)CCN3C)c1]

BBr₃ (0.044 mL, 0.463 mmol) was added to a mixture of 3-(3,5-dimethoxyphenyl)-5-(1-methyl-9-(2-methylpyridin-4-yl)-1,4,9-triazaspiro[5.5]undecan-4-yl)-1,2,4-oxadiazole (43 mg, 0.093 mmol) in DCM (2 mL) at −70° C., the resulting suspension was allowed to warm to 16° C., and stirred for 20 hours. The reaction mixture was quenched with ice-water (2 mL) at 0° C., concentrated in vacuo to provide a residue which was diluted with MeCN (3 mL), and purified directly by prep-HPLC (Column: Waters XSELECT C18 150×30 mm×5 um, condition: water (0.1% TFA)-ACN, begin B 0%, end B 30%, gradient time (min): 10, 100% B hold time (min) 1. Flow rate (ml/min): 25.), and lyophilized to provide 5-(5-(1-methyl-9-(2-methylpyridin-4-yl)-1,4,9-triazaspiro[5.5]undecan-4-yl)-1,2,4-oxadiazol-3-yl)benzene-1,3-diol. MS ESI calcd. for (C₂₃H₂₉N₆O₃) [M+H]⁺, 437.2, found, 437.2. ¹H NMR (400 MHz, CD₃OD): δ 8.06 (d, J=7.1 Hz, 1H), 7.05-7.08 (m, 2H), 6.87 (d, J=2.2 Hz, 2H), 6.41 (t, J=2.2 Hz, 1H) 4.18 (dd, J=8.4, 5.3 Hz, 4H) 4.04 (s, 2H) 3.53-3.65 (m, 4H) 2.98 (s, 3H) 2.54 (s, 3H) 2.16-2.29 (m, 4H).

### Example 8

### Preparation of Compound 107—3-(3,5-bis(oxetan-3-yloxy)phenyl)-5-(1-methyl-9-(2-methylpyridin-4-yl)-1,4,9-triazaspiro[5.5]undecan-4-yl)-1,2,4-oxadiazole

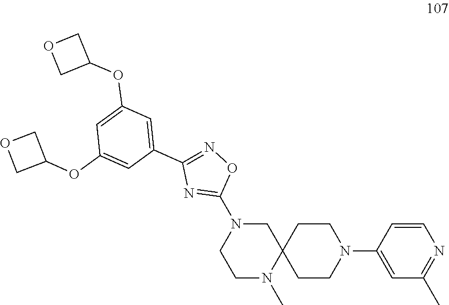 [SMILES: Cc1cc(N2CCC3(CC2)CN(c2nc(-c4cc(OC5COC5)cc(OC5COC5)c4)no2)CCN3C)ccn1]

### Step 1:

NaH (874 mg, 21.86 mmol)(60%) was added to a stirred mixture of oxetan-3-ol (1620 mg, 21.86 mmol) in THF (25 mL), and the reaction was allowed to stir at room temperature around 20° C. for 1 hour. 3,5-difluoro-4-nitrobenzonitrile (1150 mg, 6.25 mmol) was added, and the reaction was allowed to stir at room temperature around 20° C. for 8 hours. The reaction mixture was cooled to room temperature, water (50 mL) was added, and the reaction was extracted with ethyl acetate (50 mL). The combined organic extracts were washed with brine (saturated, 50 mL), dried (Na₂SO₄), filtered, and the solvent was evaporated in vacuo to provide a crude residue which was purified using flash silica gel chromatography (ISCO; 4 g Agela Silica Flash Column, Eluent of 0-50% EtOAc/PE gradient @ 30 mL/min) to provide 4-nitro-3,5-bis(oxetan-3-yloxy)benzonitrile. ¹H NMR (400 MHz, CDCl₃) δ 6.52 (s, 2H), 5.32-5.25 (m, 2H), 4.97 (dd, J=6.8, 7.7 Hz, 4H), 4.77-4.72 (m, 4H).

### Step 2:

4-nitro-3,5-bis(oxetan-3-yloxy)benzonitrile (4.2 g, 14.37 mmol) was added to a mixture of Pd/C (3.06 g, 2.87 mmol)(10%, wet) in EtOH (40 mL), and THF (40 mL). The resulting suspension was degassed and backfilled with H₂ for three times, and then stirred at 20° C. under H₂ 15 psi (hydrogen balloon) for 40 minutes. The reaction mixture was passed through a pad of Celite™ (diatomaceous earth) and the filtrate was concentrated in vacuo to provide 4-amino-3,5-bis(oxetan-3-yloxy)benzonitrile, which was used without further purification. MS ESI calc'd for C₁₃H₁₄N₂O₄ [M+H]⁺: 263.1, found: 263.2.

### Step 3:

tert-butyl nitrite (2.469 mL, 20.59 mmol) was added to a stirred mixture of 4-amino-3,5-bis(oxetan-3-yloxy)benzonitrile (3.6 g, 13.73 mmol) in DMF (30 mL), and the reaction was heated with stirring at 60° C. for 3 hours The reaction mixture was cooled to room temperature, water (50 mL) was added, and the reaction was extracted with ethyl acetate (2×20 mL). The combined organic extracts were washed with brine (saturated, 100 mL), dried (Na₂SO₄), filtered, and the solvent was evaporated in vacuo to provide a crude residue which was purified using flash silica gel chromatography (ISCO; 12 g Agela Silica Flash Column, Eluent of 0-30% EtOAc/Petroleum ether gradient @ 30 mL/min) to provide 3,5-bis(oxetan-3-yloxy)benzonitrile. ¹H NMR (400 MHz, CDCl₃) δ 6.54 (d, J=2.2 Hz, 2H), 6.34 (t, J=2.2 Hz, 1H), 5.21-5.14 (m, 2H), 4.98 (t, J=6.8 Hz, 4H), 4.73 (dd, J=5.3, 7.9 Hz, 4H).

### Step 4:

To a solution of 3,5-bis(oxetan-3-yloxy)benzonitrile (360 mg, 1.456 mmol) in ethanol (4 mL) was added triethylamine (442 mg, 4.37 mmol), and hydroxylamine hydrochloride (132 mg, 1.893 mmol). The reaction mixture was allowed to stir at 20° C. for 12 hours under N₂. The reaction mixture was purified using preparative TLC on silica gel, eluting with DCM/MeOH=10:1(V/V) to provide (Z)—N′-hydroxy-3,5-bis(oxetan-3-yloxy)benzimidamide. MS ESI calc'd for C₁₃H₁₆N₂O₅ [M+H]⁺: 281.1, found: 281.1.

### Step 5:

Di(1H-imidazol-1-yl)methanethione (69.9 mg, 0.392 mmol) was added to a stirred mixture of (Z)—N-hydroxy-3,5-bis(oxetan-3-yloxy)benzimidamide (100 mg, 0.357 mmol), and DBU (0.059 mL, 0.392 mmol) in dioxane (1 mL), and the reaction was allowed to stir at 100° C. for 2 hours. The reaction mixture was cooled to room temperature, MeI (0.112 mL, 1.784 mmol) was added, and the reaction was allowed to stir at 15° C. for 12 hours. The reaction mixture was purified using preparative TLC on silica gel, eluting with petroleum ether/EtOAc=2:1(V/V) to provide 3-(3,5-bis(oxetan-3-yloxy)phenyl)-5-(methylthio)-1,2,4-oxadiazole. MS ESI calc'd for C₁₅H₁₆N₂O₅S [M+MeCN+H]⁺: 378.0, found: 378.0.

### Step 6:

mCPBA (224 mg, 1.041 mmol)(80%) was added to a stirred mixture of 3-(3,5-bis(oxetan-3-yloxy)phenyl)-5-(methylthio)-1,2,4-oxadiazole (70 mg, 0.208 mmol) in DCM (1 mL), and the reaction was allowed to stir at 15° C. for 12 hours. The reaction mixture was filtered, and the solvent was evaporated in vacuo to provide 3-(3,5-bis(oxetan-3-yloxy)phenyl)-5-(methylsulfonyl)-1,2,4-oxadiazole, which was used without further purification.

### Step 7:

Et₃N (0.068 mL, 0.489 mmol) was added to a stirred mixture of 1-methyl-9-(2-methylpyridin-4-yl)-1,4,9-triazaspiro[5.5]undecane (46.7 mg, 0.179 mmol), K₂CO₃ (67.5 mg, 0.489 mmol), and 3-(3,5-bis(oxetan-3-yloxy)phenyl)-5-(methylsulfonyl)-1,2,4-oxadiazole (60 mg, 0.163 mmol) in DMF (1 mL), and the reaction was allowed to stir at 15° C. for 2 hours. The reaction mixture was then directly purified using HPLC (Column Waters Xbridge Prep OBD C18 150×30 5 u, condition water (0.04% NH₃—H₂O+10 mM NH₄HCO₃)-ACN, begin B 24%, end B 54%, gradient time (min) 10, 100% B hold time (min): 2. Flow rate (ml/min): 25.) to provide 3-(3,5-bis(oxetan-3-yloxy)phenyl)-5-(1-methyl-9-(2-methylpyridin-4-yl)-1,4,9-triazaspiro[5.5]undecan-4-yl)-1,2,4-oxadiazole. MS ESI calc'd for C₂₉H₃₆N₆O₅ [M+H]⁺: 549.5, found: 549.5. ¹H NMR (400 MHz, CD₃OD) δ 8.00 (d, J=6.1 Hz, 1H), 6.92 (d, J=2.2 Hz, 2H), 6.78-6.70 (m, 2H), 6.37 (t, J=2.4 Hz, 1H), 5.32-5.26 (m, 2H), 5.00 (t, J=6.8 Hz, 4H), 4.67 (dd, J=5.0, 7.2 Hz, 4H), 3.84-3.73 (m, 6H), 3.35-3.33 (m, 2H), 2.87 (t, J=5.0 Hz, 2H), 2.42-2.39 (m, 6H), 2.06-1.98 (m, 2H), 1.71-1.67 (m, 2H).

### Example 9

### Preparation of Compound 108—4-(1-(3,5-dimethoxyphenyl)pyrrolidin-3-yl)-1-methyl-9-(2-methylpyridin-4-yl)-1,4,9-triazaspiro[5.5]undecane

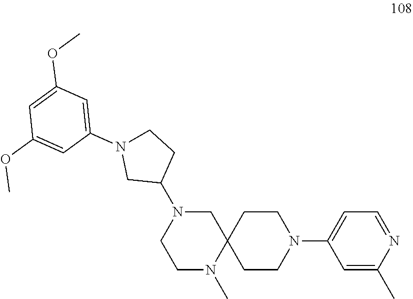 [SMILES: COc1cc(OC)cc(N2CCC(N3CCN(C)C4(CCN(c5ccnc(C)c5)CC4)C3)C2)c1]

### Step 1:

To a solution of 1-methyl-9-(2-methylpyridin-4-yl)-1,4,9-triazaspiro[5.5]undecane (100 mg, 0.384 mmol), and tert-butyl 3-oxopyrrolidine-1-carboxylate (78 mg, 0.422 mmol) in EtOH (2 mL) was added MgSO₄ (102 mg, 0.845 mmol), and AcOH (0.110 mL, 1.920 mmol). The reaction mixture was allowed to stir at 25° C. for 0.5 hours. Then NaBH₃CN (48.3 mg, 0.768 mmol) was added. The result reaction mixture was allowed to stir at 25° C. for 16 hours. The reaction mixture was diluted with NaHCO₃(aq, 5 mL), and extracted with EtOAc (10 mL×3. The organic layers were dried over Na₂SO₄, filtered, and concentrated in vacuo to provide tert-butyl 3-(1-methyl-9-(2-methylpyridin-4-yl)-1,4,9-triazaspiro[5.5]undecan-4-yl)pyrrolidine-1-carboxylate as colorless oil, which was used without further purification. MS ESI calc'd for C₂₄H₄₀N₅O₂ [M+H]⁺: 430.3, found: 430.3.

### Step 2:

To a solution of tert-butyl 3-(l-methyl-9-(2-methylpyridin-4-yl)-1,4,9-triazaspiro[5.5]undecan-4-yl)pyrrolidine-t-carboxylate (110 mg, 0.256 mmol) in DCM (2 mL) was added TFA (2 mL), and stirred at 25° C. for 0.5 hours. The reaction mixture was concentrated in vacuo and purified using HPLC to provide 1-methyl-9-(2-methylpyridin-4-yl)-4-(pyrrolidin-3-yl)-1,4,9-triazaspiro[5.5]undecane. MS ESI calc'd for C₁₉H₃₂N₅ [M+H]⁺: 330.3, found: 330.3.

### Step 3:

To a solution of 1-methyl-9-(2-methylpyridin-4-yl)-4-(pyrrolidin-3-yl)-1,4,9-triazaspiro[5.5]undecane (70 mg, 0.212 mmol) in dioxane (2 mL) was added Cs₂CO₃ (208 mg, 0.637 mmol), l-iodo-3,5-dimethoxybenzene (61.7 mg, 0.234 mmol), and methanesulfonato(2-dicyclohexylphosphino-2′,6′-diisopropyl-1,1′-biphenyl)(2′-amino-1,1′-biphenyl-2-yl)palladium(II) (44.4 mg, 0.053 mmol). The reaction mixture was allowed to stir at 100° C. for 16 hours under N₂. LCMS showed that the reaction was completed. The crude residue obtained was filtered, and purified using prep-HPLC (TFA, Column Phenomenex Synergi C18 150×30 mm×4 um, condition water (0.1% TFA)-ACN, begin B 13%, end B 33%, gradient time (min): 10. 100% B hold time (min): 2. Flow Rate (ml/min): 25.) to provide 4-(1-(3,5-dimethoxyphenyl)pyrrolidin-3-yl)-1-methyl-9-(2-methylpyridin-4-yl)-1,4,9-triazaspiro[5.5]undecane. MS ESI calc'd. for C₂₇H₄₀N₅O₂ [M+H]⁺, 466.2. found 466.4. ¹H NMR (400 MHz, DMSO-d6) δ 7.98. (d, J=6.1 Hz, 1H), 6.72-6.58 (m, 2H), 5.77 (d, J=1.8 Hz, 1H), 5.67 (s, 1H), 3.82-3.69 (m, 2H), 3.69-3.57 (m, 8H), 3.21-2.78 (m, 5H), 2.67-2.52 (m, 3H), 2.30 (s, 4H), 2.14 (s, 4H), 1.84-1.48 (m, 5H).

### Example 10

### Preparation of Compound 109—6-(3-(3,5-dichlorophenyl)cyclopent-2-en-1-yl)-2-(2-methylpyridin-4-yl)-2,6-diazaspiro[3.5]nonane

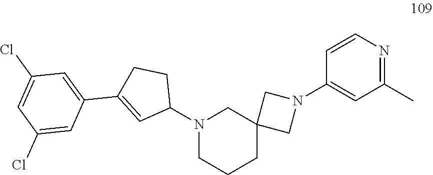 [SMILES: Cc1cc(N2CC3(CCCN(C4C=C(c5cc(Cl)cc(Cl)c5)CC4)C3)C2)ccn1]

### Step 1:

Palladium(II) acetate (18 mg, 0.078 mmol), and antimony(III) chloride (18 mg, 0.078 mmol) were added to solution of 2-cyclorenten-1-one (65 mg, 0.79 mmol), (3,5-dichlorophenyl)boronic acid (150 mg, 0.78 mmol), and sodium acetate (129 mg, 1.57 mmol) in 8 mL of acetic acid under N₂ atmosphere. After stirring for 24 hours at 25° C., the reaction mixture was filtered, and the filtrate was diluted with brine (50 mL), and then extracted twice with methylene chloride (50 mL). The organic extract was allowed to stir with saturated aqueous NaHCO₃ solution for 30 minutes, then washed with brine and dried over MgSO₄. The organic solvent was removed in vacuo and the resulting residue was purified using silica gel chromatography to provide 3-(3,5-dichlorophenyl)cyclopentan-1-one and 3-(3,5-dichlorophenyl)cyclopent-2-en-1-one, which was used without further purification.

### Step 2:

To a stirred solution of 2-(2-methylpyridin-4-yl)-2,6-diazaspiro[3.5]nonane (20 mg, 0.092 mmol) in CH₃OH (1 mL) was added triethylamine (35 mg, 0.35 mmol), and the reaction was allowed to stir at room temperature for 30 min following by 3-(3,5-dichlorophenyl)cyclopent-2-en-1-one (20 mg, 0.09 mmol). To the resulting mixture was added the solution of ZnCl₂ (0.19 mL, 0.19 mmol, 1M in Et₂O), and NaBH₃CN (32 mg, 0.44 mmol) in CH₃OH (1 mL), and the resulting reaction was allowed to stir at room temperature overnight. The organic solvent was removed in vacuo and the resulting residue was purified using preparative reversed phase HPLC to provide 6-(3-(3,5-dichlorophenyl)cyclopent-2-en-1-yl)-2-(2-methylpyridin-4-yl)-2,6-diazaspiro[3.5]nonane. MS ESI calc'd. for C₂₄H₂₈Cl₂N₃ [M+H]⁺, 428.1. found 428.0. ¹H NMR (400 MHz, MeOD) δ: 8.00-7.92 (m, 1H), 7.56 (s, 1H), 7.45 (d, J=3.6 Hz, 1H), 7.25 (s, 1H), 6.60-6.45 (m, 3H), 4.33-4.30 (m, 1H), 4.10-3.95 (m, 4H), 3.20-2.89 (m, 4H), 2.60-2.45 (m, 4H), 2.20-1.85 (m, 7H).

### Example 11

### Preparation of Compound 110—2-(3,5-dichlorophenyl)-2-methyl-1-(2-(2-methylpyridin-4-yl)-2,6-diazaspiro[3.5]nonan-6-yl)propan-1-one

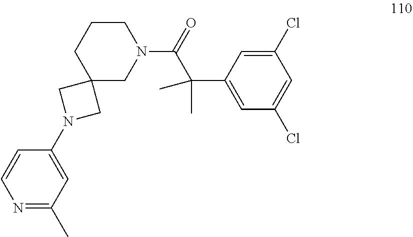 [SMILES: Cc1cc(N2CC3(CCCN(C(=O)C(C)(C)c4cc(Cl)cc(Cl)c4)C3)C2)ccn1]

To a solution of 2-(3,5-dichlorophenyl)-2-methylpropanoyl chloride (46 mg, 0.183 mmol), 2-(2-methylpyridin-4-yl)-2,6-diazaspiro[3.5]nonane (60 mg, 0.274 mmol), Et₃N (74 mg, 0.732 mmol), and HOBt (37 mg, 0.274 mmol) in DMF (2 mL) was added EDC-HCl (70 mg, 0.366 mmol). Afterwards the reaction mixture was allowed to stir at room temperature overnight, then it was partitioned between water and EtOAc. The organic layer was washed with water and dried over anhydrous Na₂SO₄. After filtration and concentration in vacuo, the residue was purified using preparative reversed phase HPLC to provide 2-(3,5-dichlorophenyl)-2-methyl-1-(2-(2-methylpyridin-4-yl)-2,6-diazaspiro[3,5]nonan-6-yl)propan-1-one. ¹H NMR (400 MHz, CDCl₃) δ 8.77 (d, J=2.0 Hz, 1H), 7.86 (m, J=8.4, 2.4 Hz, 1H), 7.29 (m, J=8.4 Hz, 2H), 7.07 (d, J=8.4 Hz, 1H), 6.97 (m, 2H), 6.77 (d, J=10.4 Hz, 1H), 6.71 (d, J=2.4 Hz, 1H), 6.46 (d, J=8.4 Hz, 1H), 6.23 (s, 1H), 5.22 (m, J=6.0 Hz, 1H), 2.09 (m, 7H), 1.51 (m, 3H), 0.53 (t, J=7.2 Hz, 6H).

### Example 12

### Preparation of Compound 111—6-(3-(3-bromophenyl)-1H-pyrazol-5-yl)-2-(2-methylpyridin-4-yl)-2,6-diazaspiro[3.5]nonane

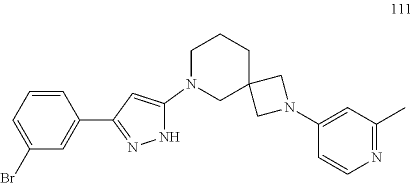 [SMILES: Cc1cc(N2CC3(CCCN(c4cc(-c5cccc(Br)c5)n[nH]4)C3)C2)ccn1]

### Step 1:

To a solution of 1-(3-bromophenyl)ethan-1-one (1.0 g, 5.02 mmol) in DMSO (10 mL) was added CS₂ (0.382 g, 5.02 mmol), NaOH (0.21 mg, 5.02 mmol, 30% aqueous solution), and Me₂SO₄ (0.634 g, 5.02 mmol). The reaction mixture was allowed to stir at room temperature for 4 hours. Ice water was then added to quench the reaction, and the reaction mixture was extracted with ethyl acetate (3×10 mL), washed with brine (20 mL), concentrated in vacuo, and the residue was purified using flash chromatographic on silica gel (PE:EtOAc=20:1 to 5:1) to provide 1-(3-bromophenyl)-3,3-bis(methylthio)prop-2-en-1-one which was used without further purification.

### Step 2:

To a solution of 1-(3-bromophenyl)-3,3-bis(methylthio)prop-2-en-1-one (100 mg, 0.33 mmol), and 2-(2-methylpyridin-4-yl)-2,6-diazaspiro[3.5]nonane (96 mg, 0.33 mmol) in CH₃CN was added K₂CO₃ (92 mg, 0.66 mmol, 2 eq), the mixture was allowed to stir at 90-100° C. for 6 hours. The reaction mixture was filtered, concentrated in vacuo to provide a crude residue, which was used without further purification.

### Step 3:

To a solution of 1-(3-bromophenyl)-3-(2-(2-methylpyridin-4-yl)-2,6-diazaspiro[3.5]nonan-6-yl)-3-(methylthio)prop-2-en-1-one (110 mg, 0.23 mmol) in ethanol (4 mL) was added H₂NNH₂.H₂O (70 mg, 1.40 mmol, 6 eq), the mixture was allowed to stir at 90-100° C. for 3 hours. The solvent was removed under pressure to provide a crude residue, which was purified using reversed phase PREP-HPLC to provide 6-(3-(3-bromophenyl)-1H-pyrazol-5-yl)-2-(2-methylpyridin-4-yl)-2,6-diazaspiro[3.5]nonane MS ESI calc'd. for C₂₂H₂₅BrN₅ [M+H]⁺, 438.1. found 438.0. ¹H NMR (400 MHz, CD₃OD) δ: 7.91 (d, J=7.2 Hz, 1H), 7.85 (s, 1H), 7.63 (d, J=8.0 Hz, 1H), 7.49 (d, J=7.2 Hz, 11H), 7.32 (t, J=8.0 Hz, 1H), 6.53 (m, 2H), 3.95 (m, 4H), 3.38 (s, 2H), 3.12 (t, J=5.6 Hz, 3H), 2.46 (s, 3H), 1.88 (m, 2H), 1.76 (m, 2H).

Compound 112 (6-(3-cyclobutyl-1H-pyrazol-5-yl)-2-(2-methylpyridin-4-yl)-2,6-diazaspiro[3.5]nonane) was prepared using the method described above for Compound 111:

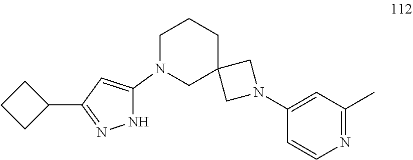 [SMILES: Cc1cc(N2CC3(CCCN(c4cc(C5CCC5)n[nH]4)C3)C2)ccn1]

MS ESI calc'd, for C₂₀H₂₈N₅ [M+H]⁺, 338.1. found 338.0. ¹H NMR (400 MHz, CD₃OD) δ:7.93 (d, J=7.2 Hz, 1H), 6.52 (m, 2H), 3.96 (s, 4H), 3.54 (m, 1H), 3.48 (s, 2H), 3.25 (t, J=4.2 Hz, 2H), 2.49 (s, 3H), 2.38 (br, 2H), 2.23 (m, 3H), 1.94 (t, J=5.6 Hz, 3H), 1.77 (br, 2H).

### Example 13

### Preparation of Compound 113—5,7-dichloro-2-(2-(2-methylpyridin-4-yl)-2,6-diazaspiro[3.5]nonan-6-yl)benzo[d]thiazole

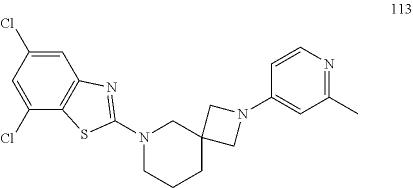 [SMILES: Cc1cc(N2CC3(CCCN(c4nc5cc(Cl)cc(Cl)c5s4)C3)C2)ccn1]

To a solution of 2,5,7-trichlorobenzo[d]thiazole (30 mg, 0.13 mmol) in CHCl₃ (1 mL) was added DIEA (84 mg, 0.65 mmol), 2-(2-methylpyridin-4-yl)-2,6-diazaspiro[3.5]nonane (34 mg, 0.16 mmol). The mixture solution was allowed to stir at 80-90° C. overnight. The crude product was purified using reversed phase prep-HPLC to provide 5,7-dichloro-2-(2-(2-methylpyridin-4-yl)-2,6-diazaspiro[3.5]nonan-6-yl)benzo[d]thiazole. MS ESI calc'd. for C₂₀H₂₁Cl₂N₄S [M+H]⁺, 419.1. found 419.0. ¹H NMR (400 MHz, CDCl₃, ppm): δ 7.85 (s, 1H), 7.35 (s, 1H), 7.05 (s, 1H), 6.35-6.45 (m, 1H), 3.80-4.00 (m, 6H), 3.25-3.35 (m, 2H), 2.35 (s, 3H), 1.85-1.95 (m, 2H), 1.15-1.35 (m, 2H).

### Example 14

### Preparation of Compound 114—2-(5-(2-(2-methylpyridin-4-yl)-2,6-diazaspiro[3.5]nonan-6-yl)-1H-pyrazol-3-yl)propan-2-ol

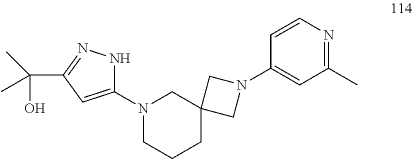 [SMILES: Cc1cc(N2CC3(CCCN(c4cc(C(C)(C)O)n[nH]4)C3)C2)ccn1]

### Step 1:

A solution of (COCl)₂ (15 mL, 0.228 mol) in CH₂Cl₂ (70 mL), and a drop of anhydrous DMF was added to a stirred solution of 2-oxopropanoic acid (10 g, 0.114 mol) in CH₂Cl (80 mL). The reaction mixture was allowed to stir at room temperature until gas evolution ceased. The reaction mixture was concentrated in vacuo and the resulting residue was dissolved in CH₂Cl₂ (40 mL) then added to a stirred solution of anhydrous pyridine (16.2 g, 0.205 mol), and t-Butanol (8.88 g, 0.12 mol) in CH₂Cl₂ (50 mL). The reaction mixture was allowed to stir at room temperature for 20 hours. After completion of the reaction by TLC, the mixture was washed with water (150 mL). The combined organic layers were concentrated in vacuo and purified using a flash chromatography on silica gel to provided tert-butyl 2-oxopropanoate which was used in step 2.

### Step 2:

A three-necked flask is charged with t-BuOK (3.1 g, 27.7 mmol), tert-butyl 2-oxopropanoate (2.0 g, 3.9 mmol), and anhydrous THF (60 mL). After 15 min, CS₂ (1.05 g, 13.9 mmol) is added dropwise, taking caution not to allow the mixture to warm significantly. CH₃I (3.94 g, 27.7 mol) was added in a similar fashion and the mixture stirred at room temperature for 10 hours. The resultant heterogeneous mixture was poured onto ice (100 g), and extracted with EtOAc (30 mL×3). The combined organic layers were dried over anhydrous. Na₂SO₄, filtered, concentrated in vacuo and purified using a flash chromatography on silica gel to provide the tert-butyl 4,4-bis(methylthio)-2-oxobut-3-enoate which was used in step 3.

### Step 3:

A solution of tert-butyl 4,4-bis(methylthio)-2-oxobut-3-enoate (150 mg, 0.605 mmol), 2-(2-methylpyridin-4-yl)-2,6-diazaspiro[3.5]nonane (138 mg, 0.54 mmol), and K₂CO₃ (187 mg, 1.36 mmol) in CH₃CN (4 mL) was heated to reflux for 20 hours under N₂. After the completion of the reaction by TLC, the reaction mixture was concentrated in vacuo to provide a crude residue, tert-butyl 4-(2-(2-methylpyridin-4-yl)-2,6-diazaspiro[3.5]nonan-6-yl)-4-(methylthio)-2-oxobut-3-enoate which was used directly in step 4.

### Step 4:

A solution of tert-butyl 4-(2-(2-methylpyridin-4-yl)-2,6-diazaspiro[3.5]nonan-6-yl)-4-(methylthio)-2-oxobut-3-enoate (30 mg, 0.071 mmol), NH₂ NH₂H₂O (1.07 mg, 0.021 mmol) in EtOH (1 mL) was heated to reflux for 2 hours under N₂. After the completion of the reaction by TLC, the reaction mixture was filtered, washed with CH₃OH (5 mL), concentrated in vacuo and purified using reversed phase HPLC to provide tert-butyl 5-(2-(2-methylpyridin-4-yl)-2,6-diazaspiro[3.5]nonan-6-yl)-1H-pyrazole-3-carboxylate which was used directly in step 5.

### Step 5:

A solution of tert-butyl 5-(2-(2-methylpyridin-4-yl)-2,6-diazaspiro[3.5]nonan-6-yl)-1H-pyrazole-3-carboxylate (30 mg, 0.078 mmol) in 4 mL of HCl(g)/CH₃OH (6 mol/L, 24 mmol) was heated to reflux for 3 hours. After the completion of the reaction, the reaction mixture was concentrated then purified using reversed phase HPLC to provide methyl 5-(2-(2-methylpyridin-4-yl)-2,6-diazaspiro[3.5]nonan-6-yl)-1H-pyrazole-3-carboxylate which was used directly in step 6.

### Step 6:

A solution of MeLi (1.6 M in Et₂O, 0.22 mL, 0.352 mmol) was added by syringe to a solution of 5-(2-(2-methylpyridin-4-yl)-2,6-diazaspiro[3.5]nonan-6-yl)-H-pyrazole-3-carboxylate (20 mg, 0.058 mmol) in anhydrous THF (1 mL) at −78° C. After 45 min, the reaction was quenched with H₂O and extracted by EtOAc (4 mL×2). The combined the organic layers were dried over Na₂SO₄, filtered, concentrated and purified using reversed phase HPLC to provide 2-(5-(2-(2-methylpyridin-4-yl)-2,6-diazaspiro[3.5]nonan-6-yl)-1H-pyrazol-3-yl)propan-2-ol. ¹H NMR (400 MHz, CD₃OD) δ: 7.96 (d, J=7.2 Hz, 1H), 6.51-6.56 (m, 2H), 3.98-4.00 (m, 4H), 3.48 (s, 2H), 3.26 (m, 2H), 2.47 (s, 3H), 1.94 (br, 2H), 1.79 (br, 2H), 1.55 (s, 611).

### Example 15

### Preparation of Compound 115—3-(3-chlorophenyl)-5-(2-(2-methylpyridin-4-yl)-2,6-diazaspiro[3.5]nonan-6-yl)isoxazole

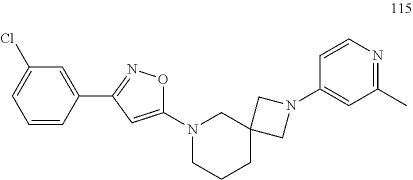 [SMILES: Cc1cc(N2CC3(CCCN(c4cc(-c5cccc(Cl)c5)no4)C3)C2)ccn1]

### Step 1:

A solution of 1-(3-chlorophenyl)-3,3-bis(methylthio)prop-2-en-1-one (100 mg, 0.39 mmol), 2-(2-methylpyridin-4-yl)-2,6-diazaspiro[3.5]nonane (84 mg, 0.39 mmol) in CHCN (2 mL) was heated to reflux for 13 hours under N₂. After the completion of the reaction, the reaction mixture was concentrated in vacuo to provide a crude residue, 1-(3-chlorophenyl)-3-(2-(2-methylpyridin-4-yl)-2,6-diazaspiro[3.5]nonan-6-yl)-3-(methylthio)prop-2-en-1-one which was used for the next step directly.

### Step 2:

NH₂OH.HCl (84 mg, 1.2 mmol) was added to a solution of CH₃ONa (58 mg, 1.08 mmol) in anhydrous CH₃OH (2 mL), and the reaction was allowed to stir at 70-80° C. for 3 hours. 1-(3-chlorophenyl)-3-(2-(2-methylpyridin-4-yl)-2,6-diazaspiro[3.5]nonan-6-yl)-3-(methylthio)prop-2-en-1-one (79 mg, 0.18 mmol) was added, and the reaction was heated at reflux for 6 hours. After the completion of the reaction by TLC, the reaction mixture was filtered, and washed with CH₃OH (5 mL). After filtration and concentration in vacuo, the residue was purified using reversed phase HPLC to provide 3-(3-chlorophenyl)-5-(2-(2-methylpyridin-4-yl)-2,6-diazaspiro[3.5]nonan-6-yl)isoxazole. 1H NMR (400 MHz, CD₃OD) δ: 7.92 (d, J=7.2 Hz, 1H), 7.76 (s, 1H), 7.68 (m, 1H), 6.73 (s, 1H), 6.51 (m, 2H), 3.93-4.01 (m, 4H), 3.48 (s, 2H), 2.46 (s, 3H), 1.92 (br, 2H), 1.75 (br, 2H).

### Example 16

### Preparation of Compound 116—3-(3,5-dichlorophenyl)-5-(2-(2-methylpyridin-4-yl)-2,6-diazaspiro[3.5]nonan-6-yl)-1,2,4-oxadiazole

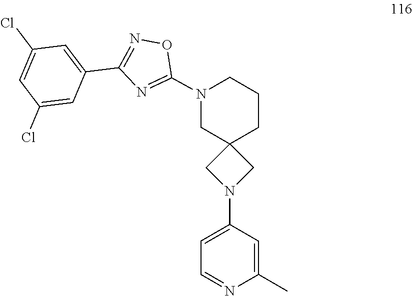 [SMILES: Cc1cc(N2CC3(CCCN(c4nc(-c5cc(Cl)cc(Cl)c5)no4)C3)C2)ccn1]

A solution of 5-bromo-3-(3,5-dichlorophenyl)-1,2,4-oxadiazole (70 mg, 0.238 mmol), 2-(2-methylpyridin-4-yl)-2,6-diazaspiro[3.5]nonane (52 mg, 0.238 mmol), and K₂CO₃ (65.8 mg, 0.476 mmol) in DMF (0.85 mL) was allowed to stir at room temperature for 12 hours. The solution was diluted with ethyl acetate, washed with water and brine solution, dried over Na₂SO₄, filtered, and concentrated in vacuo. The residue was then purified using chromatography on silica gel to provide 3-(3,5-dichlorophenyl)-5-(2-(2-methylpyridin-4-yl)-2,6-diazaspiro[3.5]nonan-6-yl)-1,2,4-oxadiazole. MS ESI calc'd. for C₂₁H₂₂Cl₂N₅O [M+H]≡⁶ 431.1, found 431. 1H NMR (400 MHz, CD₃OD): 7.93 (d, J=6.8 Hz, 1H), 7.81 (d, J=1.6 Hz, 2H), 7.54 (s, 1H), 6.46-6.51 (m, 2H), 3.89-4.01 (m, 6H), 3.65 (t, J=5.6 Hz, 2H), 2.46 (s, 3H), 1.99 (t, J=5.6 Hz, 2H), 1.77 (m, 2H).

The following compounds were prepared using the methods disclosed in Example 16:

| No. | | [M + H]+ Obs'd |
|---|---|---|
| 117 | 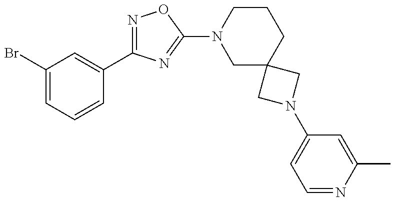 [SMILES: Cc1cc(N2CC3(CCCN(c4nc(-c5cccc(Br)c5)no4)C3)C2)ccn1] | 442 |
| 118 | 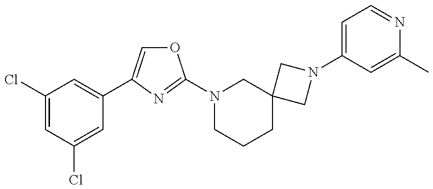 [SMILES: Cc1cc(N2CC3(CCCN(c4nc(-c5cc(Cl)cc(Cl)c5)co4)C3)C2)ccn1] | 429 |
| 119 | 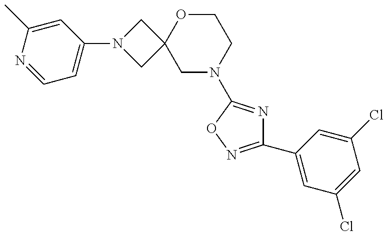 [SMILES: Cc1cc(N2CC3(C2)CN(c2nc(-c4cc(Cl)cc(Cl)c4)no2)CCO3)ccn1] | 432 |
| 120 | 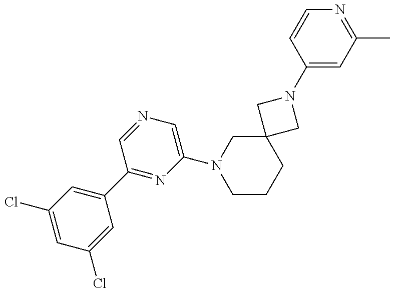 [SMILES: Cc1cc(N2CC3(CCCN(c4cncc(-c5cc(Cl)cc(Cl)c5)n4)C3)C2)ccn1] | 440 |

### Example 17

### Preparation of Compound 121—3-(3,5-dichlorophenyl)-5-(2-(2-methylpyridin-4-yl)-2,6-diazaspiro[3.5]nonan-6-yl)-1,2,4-isoxazole

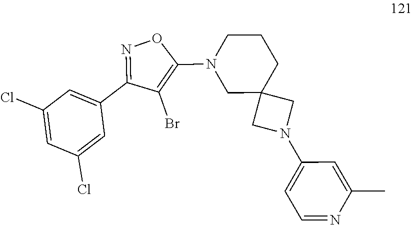 [SMILES: Cc1cc(N2CC3(CCCN(c4onc(-c5cc(Cl)cc(Cl)c5)c4Br)C3)C2)ccn1]

To a solution of compound 3-(3,5-dichlorophenyl)-5-(2-(2-methylpyridin-4-yl)-2,6-diazaspiro[3.5]nonan-6-yl)isoxazole (2 g, 4.6 mmol) in TFA (10 mL) was added NBS (1 g, 5.6 mmol). The reaction mixture was allowed to stir overnight and the solvent was evaporated. The crude product was purified using silica gel chromatography (DCM:MeOH=30:1) to provide 4-bromo-3-(3,5-dichlorophenyl)-5-(2-(2-methylpyridin-4-yl)-2,6-diazaspiro[3.5]nonan-6-yl)isoxazole. MS ESI calc'd. for C₂₂H₂₃BrCl₂N₄O [M+H]⁺ 509.1, found 509.5. ¹H NMR (400 MHz, CD₃OD) δ (ppm): 7.93 (d, J=6.4 Hz, 1H), 7.87 (s, 2H), 7.61 (s, 1H), 6.48 (d, J=6.4 Hz, 1H), 6.46 (s, 1H), 4.04 (d, 2H), 3.91 (d, 2H), 3.48 (s, 2H), 3.3 (s, 2H), 2.45 (s, 3H), 1.91 (t, 2H), 1.78 (t, 2H).

### Example 18

### Preparation of Compound 122—3-(3,5-dichlorophenyl)-4-(furan-3-yl)-5-(2-(2-methylpyridin-4-yl)-2,6-diazaspiro[3.5]nonan-6-yl)isoxazole

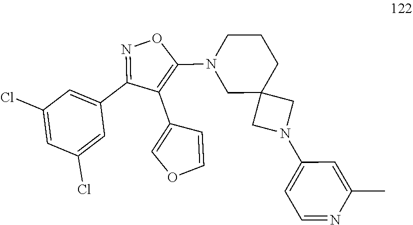 [SMILES: Cc1cc(N2CC3(CCCN(c4onc(-c5cc(Cl)cc(Cl)c5)c4-c4ccoc4)C3)C2)ccn1]

Into 5 mL of toluene: H₂O (5:1) in a 10 mL of round bottom flask equipped with a stirring bar was bubbled in a stream of argon for 30 minutes. Pd(PPh₃)₄(12 mg, 0.01 mmol), and 4-bromo-3-(3,5-dichlorophenyl)-5-(2-(2-methylpyridin-4-yl)-2,6-diazaspiro[3.5]nonan-6-yl)isoxazole (60 mg, 0.11 mmol) were added into this solvent mixture, and the resulting mixture was allowed to stir at room temperature under argon for 1 hour. Furan-3-ylboronic acid (13 mg, 0.12 mmol) in 0.5 mL of EtOH and Na₂CO₃ (60 mg) was added into the reaction mixture at room temperature under argon. The reaction mixture was heated under argon with vigorous stirring at 80° C. for 8 hours. The solvent was evaporated and the crude product was purified via reversed phase HPLC to provide 3-(3,5-dichlorophenyl)-4-(furan-3-yl)-5-(2-(2-methylpyridin-4-yl)-2,6-diazaspiro[3.5]nonan-6-yl)isoxazole.

MS ESI calc'd. for C₂₆H₂₅Cl₂N₄O₂ [M+H]⁺ 495.1, found 495.4. ¹H NMR (400 MHz, CD₃OD) δ (ppm): 7.96 (d, J=6.4 Hz, 1H), 7.63 (s, 2H), 7.56 (s, 1H), 7.48 (s, 3H), 6.56 (s, 1H), 6.48 (d, J=6.4 Hz, 1H), 6.46 (s, 1H), 3.90 (d, 2H), 3.87 (d, 2H), 3.28 (s, 2H), 3.03 (s, 2H), 2.45 (s, 3H), 1.91 (t, 2H), 1.78 (t, 2H).

The following compounds were prepared using the methods disclosed in Example 18.

| Compound | | [M + H]+ |
|---|---|---|
| ID | | Obs'd |
| 123 | 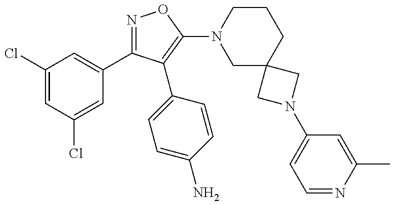 [SMILES: Cc1cc(N2CC3(CCCN(c4onc(-c5cc(Cl)cc(Cl)c5)c4-c4ccc(N)cc4)C3)C2)ccn1] | 521 |
| 124 | 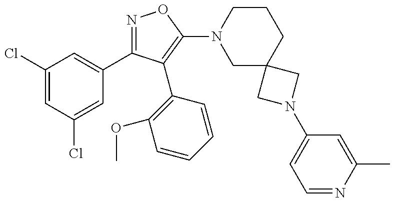 [SMILES: COc1ccccc1-c1c(-c2cc(Cl)cc(Cl)c2)noc1N1CCCC2(CN(c3ccnc(C)c3)C2)C1] | 536 |
| 125 | 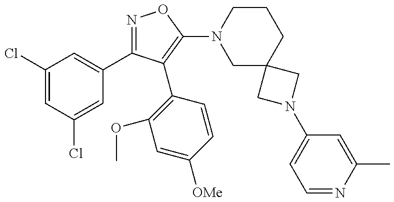 [SMILES: COc1ccc(-c2c(-c3cc(Cl)cc(Cl)c3)noc2N2CCCC3(CN(c4ccnc(C)c4)C3)C2)c(OC)c1] | 566 |

### Example 19

### Preparation of Compound 126—6-(1-(3,5-dichlorophenyl)-1H-pyrazol-3-yl)-2-(2-methylpyridin-4-yl)-2,6-diazaspiro[3.5]nonane

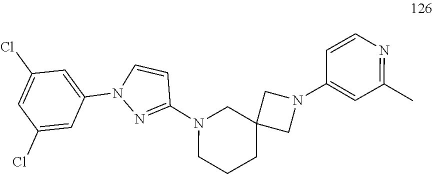 [SMILES: Cc1cc(N2CC3(CCCN(c4ccn(-c5cc(Cl)cc(Cl)c5)n4)C3)C2)ccn1]

### Step 1:

To a solution of 1-(3,5-dichlorophenyl)-1H-pyrazol-3-amine (1.9 g, 9 mmol) in HBr (10 mL, 48%), and water (10 mL) was added a solution of NaNO₂ in water (10 mL) dropwise at 0° C. The reaction mixture was allowed to stir at 0° C. for 0.5 hours. To this was added CuBr (1.3 g, 9 mmol) in HBr (5 mL, 48%), and the precipitate appeared immediately. The reaction mixture was heated to 70° C. slowly, and then stirred for 5 hrs. The resulting mixture was filtered, and washed with EtOAc. The filtrate was concentrated. To the residue was added sat. aqueous NaHCO₃, and the aqueous layer was re-extracted with ethyl acetate. The combined organic layer was dried over anhydrous Na₂SO₄, filtered, and concentrated in vacuo to provide 3-bromo-1-(3,5-dichlorophenyl)-1H-pyrazole which was used as such in step 2.

### Step 2:

A mixture of 3-bromo-1-(3,5-dichlorophenyl)-1H-pyrazole (30 mg, 0.103 mmol), 2-(2-methylpyridin-4-yl)-2,6-diazaspiro[3.5]nonane (25 g, 0.113 mmol), Pd₂(dba)₃ (5 mg, 0.005 mmol), BINAP (6 mg, 0.01 mmol), Cs₂CO₃ (101 mg, 0.303 mmol) in toluene (1.5 mL) was heated at 80° C. for 16 hours. The resulting mixture was purified using prep-TLC to provide 6-(1-(3,5-dichlorophenyl)-1H-pyrazol-3-yl)-2-(2-methylpyridin-4-yl)-2,6-diazaspiro[3.5]nonane, t H NMR (400 MHz, CDCl₃) δ (ppm): 8.06 (s, 1H), 7.67 (d, 1H), 7.43 (d, 1H₁), 7.07 (s, 1H), 6.21 (s, 1H), 6.08 (s, 1H), 5.92 (d, 1H), 3.90 (d, 2H), 3.80 (t, 2H), 3.40 (s, 2H), 3.16 (s, 2H), 2.50 (s, 3H), 1.83 (t, 2H), 1.70 (s, 2H).

### Example 20

### Preparation of Compound 127—1-(3,5-dichlorophenyl)-2-methyl-3-(2-(2-methylpyridin-4-yl)-2,6-diazaspiro[3.5]nonan-6-yl)propan-1-one

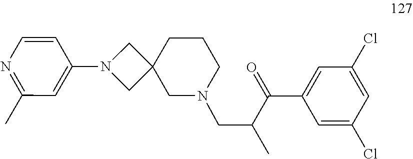 [SMILES: Cc1cc(N2CC3(CCCN(CC(C)C(=O)c4cc(Cl)cc(Cl)c4)C3)C2)ccn1]

To a solution of 2-(2-methylpyridin-4-yl)-2,6-diazaspiro[3.5]nonane (17.6 mg, 0.558 mmol), and Et₃N (15.4 mg, 0.152 mol) in DMF (2 mL), was added 2-bromo-1-(3,5-dichlorophenyl)-2-methylpropan-1-one (15 mg, 0.051 mmol). The reaction mixture was allowed to stir at room temperature overnight, then poured into ice-water and extracted with EtOAc. The organic layer was washed with brine and dried over anhydrous Na₂SO₄. After filtration and concentration in vacuo, the residue was purified using preparative reversed phase HPLC to provide 1-(3,5-dichlorophenyl)-2-methyl-3-(2-(2-methylpyridin-4-yl)-2,6-diazaspiro[3.5]nonan-6-yl)propan-1-one. ¹H NMR (400 MHz, CD₃OD): δ 7.97 (d, J=7.6 Hz, 3H), 7.76 (s, 1H), 6.52 (m, 2H), 4.19-4.07 (m, 6H), 4.01-3.79 (br, 2H), 3.21 (m, 2H), 2.47 (s, 3H), 2.25-1.26 (m, 5H), 1.25 (s, 3H).

### Example 21

### Preparation of Compound 128—6-(3-(2,3-difluorophenyl)-1H-pyrazol-5-yl)-2-(2-methylpyridin-4-yl)-2,6-diazaspiro[3.5]nonane

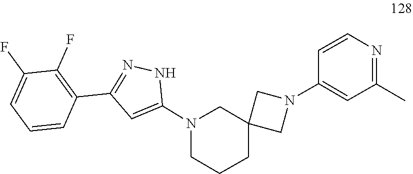 [SMILES: Cc1cc(N2CC3(CCCN(c4cc(-c5cccc(F)c5F)n[nH]4)C3)C2)ccn1]

### Step 1:

1-(2,3-difluorophenyl)-3,3-bis(methylthio)prop-2-en-1-one (50 mg, 0.19 mmol), and 2-(2-methylpyridin-4-yl)-2,6-diazaspiro[3.5]nonane (41.7 mg, 0.19 mmol) were dissolved in acetonitrile and the reaction was allowed to stir at 80 for 5 hours. The solvent was removed and the resulting residue was used in step 2 without purification.

### Step 2:

The solution of 1-(2,3-difluorophenyl)-3-(2-(2-methylpyridin-4-yl)-2,6-diazaspiro[3.5]nonan-6-yl)-3-(methylthio)prop-2-en-1-one (40 mg, 0.093 mmol) in ethanol (3 mL) was added hydrazine hydrate (52 mg, 1.0 mmol), and the reaction was heated at reflux for 1 hour. The solvent was removed to provide a crude residue, which was purified using reversed phase PREP-HPLC to provide 6-(3-(2,3-difluorophenyl)-1H-pyrazol-5-yl)-2-(2-methylpyridin-4-yl)-2,6-diazaspiro[3.5]nonane. MS ESI calc'd. for C₂₂H₂₄F₂N₅ [M+H]⁺ 396.1, found 396. 1H NMR (400 MHz, CD₃OD) δ: 7.96 (d, J=8 Hz, 1H), 7.51-7.55 (m, 1H), 7.23-7.31 (m, 2H), 6.53-6.57 (m, 2H), 3.96-4.06 (m, 4H), 3.41 (s, 2H), 3.20 (t, J=12 Hz, 2H), 2.50 (s, 3H), 1.83-1.93 (m, 2H), 1.80-1.83 (m, 2H).

### Examples 22 & 23

### Preparation of Compound 129—methyl 3-(5-(2-(2-methylpyridin-4-yl)-2,6-diazaspiro[3.5]nonan-6-yl)isoxazol-3-yl)benzoate and Compound 130—methyl 3-(5-(2-(2-methylpyridin-4-yl)-2,6-diazaspiro[3.5]nonan-6-yl)-1H-pyrazol-3-yl)benzoate

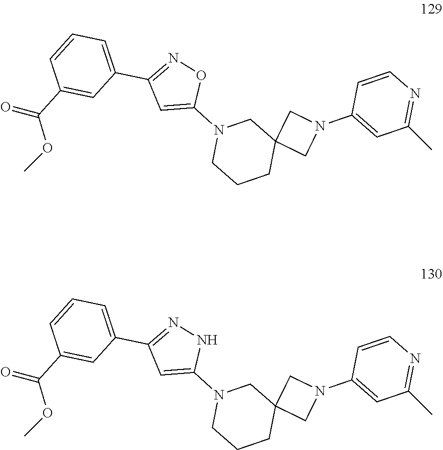 [SMILES: COC(=O)c1cccc(-c2cc(N3CCCC4(CN(c5ccnc(C)c5)C4)C3)on2)c1 ; COC(=O)c1cccc(-c2cc(N3CCCC4(CN(c5ccnc(C)c5)C4)C3)[nH]n2)c1]

### Step 1:

CS₂ (0.44 g, 5.7 mmol) was added dropwise to a stirred solution of tert-butyl 3-acetylbenzoate (1.27 g, 5.7 mmol) in DMSO (8 mL), and 30% aqueous NaOH (3.2 mL) at 0° C. Stirring was continued at 0° C. for 30 minutes. Me₂SO₄ (2.18 g, 17.3 mmol) was added dropwise to the mixture at 0° C., and continued stirring for 2 hours at 0° C. Ice water was added, and the mixture extracted with EtOAc (20 mL×3), then dried over anhydrous Na₂SO₄, filtered, and the filtrate concentrated and purified using flash chromatography on silica gel to provide tert-butyl 3-(3,3-bis(methylthio)acryloyl)benzoate.

### Step 2:

A solution of tert-butyl 3-(3,3-bis(methylthio)acryloyl)benzoate (300 mg, 0.92 mmol), 2-(2-methylpyridin-4-yl)-2,6-diazaspiro[3.5]nonane (187 mg, 0.74 mmol), and K₂CO₃ (319 mg, 2.31 mmol) in CH₃CN (6 mL) was heated to reflux for 20 hours under N₂. The reaction mixture was concentrated in vacuo to provide crude tert-butyl-3-(3-(2-(2-methylpyridin-4-yl)-2,6-diazaspiro[3.5]nonan-6-yl)-3-(methylthio)acryloyl)benzoate which was used directly in step 3 directly.

### Step 3:

NH₂OH.HCl (39 mg, 0.729 mmol) was added to a solution of CH₃ONa (39 mg, 0.729 mmol) in anhydrous CH₃OH (1.5 mL), and the reaction was allowed to stir at 70° C. for 3 hours. tert-butyl-3-(3-(2-(2-methylpyridin-4-yl)-2,6-diazaspiro[3.5]nonan-6-yl)-3-(methylthio)acryloyl)benzoate (60 mg, 0.121 mmol) was added, and the reaction was heated at reflux for 10 hours. The reaction mixture was filtered, washed with CH₃OH (6 mL), concentrated in vacuo then purified using reversed phase HPLC to provide tert-butyl 3-(5-(2-(2-methylpyridin-4-yl)-2,6-diazaspiro[3.5]nonan-6-yl)isoxazol-3-yl)benzoate.

### Step 4:

A solution of tert-butyl 3-(5-(2-(2-methylpyridin-4-yl)-2,6-diazaspiro[3.5]nonan-6-yl)isoxazol-3-yl)benzoate (30 mg, 0.065 mmol) in 3 mL of HCl(g)/CH₃OH (6 mol/L) was heated to reflux for 3 hours. The reaction mixture was concentrated in vacuo directly, then purified using HPLC to provide methyl 3-(5-(2-(2-methylpyridin-4-yl)-2,6-diazaspiro[3.5]nonan-6-yl)isoxazol-3-yl)benzoate ¹H NMR (400 MHz, CD₃OD) δ: 8.41 (s, 1H), 8.10 (d, J=7.2 Hz, 1H), 8.00 (d, J=6.8 Hz, 1H), 7.98 (d, J=6.8 Hz, 1H), 7.64 (m, 1H), 6.82 (s, 1H), 6.54-6.58 (m, 2H), 4.00-4.04 (m, 4H), 3.97 (s, 3H), 3.55 (s, 2H), 3.34 (m, 2H), 2.51 (s, 3H), 1.97 (br, 2H), 1.80 (br, 2H).

### Step 5:

A solution of tert-butyl-3-(3-(2-(2-methylpyridin-4-yl)-2,6-diazaspiro[3.5]nonan-6-yl)-3-(methylthio)acryloyl)benzoate (70 mg, 0.142 mmol), NH₂ NH₂—H₂O (50 mg, 0.451 mmol) in EtOH (2 mL) was heated to reflux for 2 hours under N₂. The reaction mixture was filtered, washed with CH₃OH (6 mL), concentrated in vacuo, then purified using reversed phase HPLC to provide tert-butyl 3-(5-(2-(2-methylpyridin-4-yl)-2,6-diazaspiro[3.5]nonan-6-yl)-1H-pyrazol-3-yl)benzoate.

### Step 6:

A solution of tert-butyl 3-(5-(2-(2-methylpyridin-4-yl)-2,6-diazaspiro[3.5]nonan-6-yl)-1H-pyrazol-3-yl)benzoate (50 mg, 0.108 mmol) in 6 mL of HCl(g)/CH₃OH (6 mol/L, 36 mmol) was heated to reflux for 3 hours. The reaction mixture was concentrated in vacuo directly, then purified using reversed phase HPLC to provide methyl 3-(5-(2-(2-methylpyridin-4-yl)-2,6-diazaspiro[3.5]nonan-6-yl)-1H-pyrazol-3-yl)benzoate. ¹H NMR (400 MHz, CD₃OD) δ: 8.32 (s, 1H), 7.98 (d, J=7.6 Hz, 1H), 7.88-7.93 (m, 2H), 7.54 (m, 1H), 6.50-6.53 (m, 2H), 3.93-4.03 (m, 4H), 3.91 (s, 3H), 3.38 (s, 2H), 3.19 (m, 2H), 2.46 (s, 3H), 1.88 (br, 2H), 1.77 (br, 2H).

### Example 24

### Preparation of Compound 131—6-(3-(3,4-difluorophenyl)-1H-pyrazol-5-yl)-2-(2-methylpyridin-4-yl)-2,6-diazaspiro[3.5]nonane

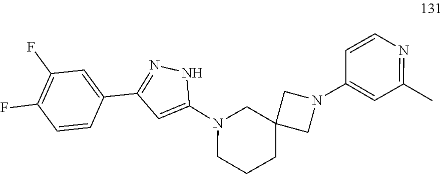 [SMILES: Cc1cc(N2CC3(CCCN(c4cc(-c5ccc(F)c(F)c5)n[nH]4)C3)C2)ccn1]

### Step 1:

To a solution of 1-(3,4-difluorophenyl)ethan-1-one (1.56 g, 10 mmol) in a solution of 30% NaOH (2.9 mL) in DMSO (25 mL) at 0° C. was added CS₂ (0.76 g, 10 mmol) slowly, the reaction mixture was allowed to stir for 30 minutes at the same temperature. Then Me₂SO₄ (3.78 g, 30 mmol) was added to the mixture slowly and the resulting mixture was allowed to stir for 2 hours at 0° C. Ice-water was added, the precipitate was collected and washed with water and petroleum ether to provide 1-(3,4-difluorophenyl)-3,3-bis(methylthio)prop-2-en-1-one, which was used in step 2.

### Step 2:

A solution of 1-(3,4-difluorophenyl)-3,3-bis(methylthio)prop-2-en-1-one (130 mg, 0.5 mmol), 2-(2-methylpyridin-4-yl)-2,6-diazaspiro[3.5]nonane (109 mg, 0.5 mmol) in CH₃CN (2 mL) was heated to reflux for 13 hours under N₂. The reaction mixture was concentrated in vacuo to provide crude 1-(3,4-difluorophenyl)-3-(2-(2-methylpyridin-4-yl)-2,6-diazaspiro[3.5]nonan-6-yl)-3-(methylthio)prop-2-en-1-one which was used without further purification.

### Step 3:

A solution of 1-(3,4-difluorophenyl)-3-(2-(2-methylpyridin-4-yl)-2,6-diazaspiro[3.5]nonan-6-yl)-3-(methylthio)prop-2-en-1-one (50 mg, 0.12 mmol), NH₂ NH₂—H₂O (11.7 mg, 0.24 mmol) in EtOH (2 mL) was heated to reflux for 3 hours under N₂. The reaction mixture was filtered, washed with CH₃OH, concentrated in vacuo, then purified using reversed phase HPLC to provide 6-(3-(3,4-difluorophenyl)-1H-pyrazol-5-yl)-2-(2-methylpyridin-4-yl)-2,6-diazaspiro[3.5]nonane. MS ESI calc'd. for C₂₂H₂₄F₂N₅ [M+H]⁺ 396.1, found 396. ¹H NMR (400 MHz, CD₃OD) δ: 7.93 (d, J=7.0 Hz, 1H), 7.60 (t, J=7.8 Hz, 1H), 7.47-7.50 (m, 1H), 7.32 (dd, J=10, 8.6 Hz, 2H), 6.49-6.53 (m, 2H), 3.92-4.01 (m, 4H), 3.38 (s, 2H), 3.17-3.19 (m, 2H), 2.47 (s, 3H), 1.87-1.90 (m, 3H), 1.76-1.77 (m, 3H).

### Example 25

### Preparation of Compound 132—6-(2-(3,5-dichlorophenyl)pyridin-4-yl)-2-(2-methylpyridin-4-yl)-2,6-diazaspiro[3.5]nonane

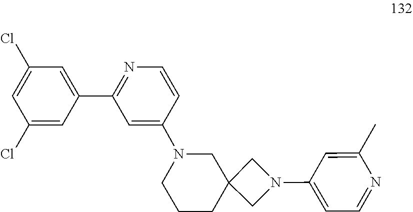 [SMILES: Cc1cc(N2CC3(CCCN(c4ccnc(-c5cc(Cl)cc(Cl)c5)c4)C3)C2)ccn1]

To a stirred solution of 4-chloro-2-(3,5-dichlorophenyl)pyridine (50.0 mg, 0.19 mmol), 2-(2-methylpyridin-4-yl)-2,6-diazaspiro[3.5]nonane (56 mg, 0.193 mmol), t-BuONa (72.1 mg, 0.77 mmol), BINAP (12 mg, 0.019 mmol) in toluene (5 mL) was added Pd₂(dba)₃ (18.0 mg, 0.019 mmol) under N₂, and the reaction was heated to 70° C. overnight under N₂ atmosphere. The organic solvent was removed in vacuo, and the resulting residue was purified using preparative reversed phase HPLC to provide 6-(2-(3,5-dichlorophenyl)pyridin-4-yl)-2-(2-methylpyridin-4-yl)-2,6-diazaspiro[3.5]nonane. MS ESI calc'd. for C₂₄H₂₅Cl₂N₄ [M+H]⁺ 439.1, found 439.4. ¹H NMR (400 MHz, CD₃OD) δ: 8.14 (m, 1H), 7.93 (d, 2H), 7.84 (s, 1H), 7.70 (s, 1H), 7.51 (s, 1H). 7.31 (s, 1H), 6.50 (d, 2H), 3.93-4.02 (m, 6H), 3.76 (s, 2H), 2.46 (s, 3H), 2.07 (s, 2H), 1.82 (s, 2H).

### Example 26

### Preparation of Compound 133—3-(2,3-difluorophenyl)-5-(2-(2-methylpyridin-4-yl)-2,6-diazaspiro[3.5]nonan-6-yl)isoxazole

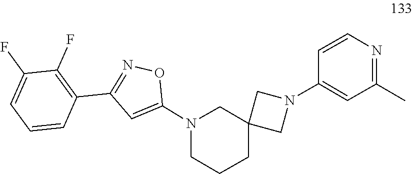 [SMILES: Cc1cc(N2CC3(CCCN(c4cc(-c5cccc(F)c5F)no4)C3)C2)ccn1]

### Step 1:

NaHMDS (1.7 mL, 3.5 mmol, 2M in THF) was added to the solution of 1-(2,3-difluorophenyl)ethan-1-one (0.55 g, 3.5 mmol) in THF (25 mL) at −78° C. and the reaction was allowed to stir at −78° C. for 1 h. Then CS₂ (0.26 g, 3.5 mmol) was added to the mixture, and stirred at −78° C. for 1 h, MeI (1 g, 7 mmol) was added, and the reaction was allowed to warm up to room temperature and stirred for 30 minutes at room temperature. The reaction was quenched with saturated aqueous NH₄Cl (22 mL). The reaction mixture was extracted with ethyl acetate (50 mL×3), washed with brine (25 mL), dried over anhydrous Na₂SO₄ and evaporated, then the residue was purified using chromatography on silica gel to provide 1-(2,3-difluorophenyl)-3,3-bis(methylthio)prop-2-en-1-one, which was used without further purification.

### Step 2:

1-(2,3-difluorophenyl)-3,3-bis(methylthio)prop-2-en-1-one (50 mg, 0.19 mmol), and 2-(2-methylpyridin-4-yl)-2,6-diazaspiro[3.5]nonane (41.7 mg, 0.19 mmol) were dissolved in acetonitrile and the reaction was allowed to stir at 80 for 5 hours. The solvent was removed and the resulting residue was used without further purification.

### Step 3:

Hydroxylamine hydrochloride (48 mg, 0.7 mmol) was added to a solution of sodium methoxide (0.349 mL, 0.7 mmol), and 1-(2,3-difluorophenyl)-3-(2-(2-methylpyridin-4-yl)-2,6-diazaspiro[3.5]nonan-6-yl)-3-(methylthio)prop-2-en-1-one (50 mg, 0.116 mmol), and the reaction was heated to reflux overnight. The solvent was removed to provide a residue which was purified using reversed phase PREP-HPLC to provide 3-(2,3-difluorophenyl)-5-(2-(2-methylpyridin-4-yl)-2,6-diazaspiro[3.5]nonan-6-yl)isoxazole MS ESI calc'd. for C₂₂H₂₃F₂N₄O [M+H]⁺ 397.1, found 397. ¹H NMR (400 MHz, CD₃OD) δ: 7.97 (d, J=8 Hz, 1H), 7.65-7.68 (m, 1H), 7.39-7.43 (m, 1H), 7.30-7.35 (m, 1H), 6.74 (s, 1H), 6.53-6.57 (m, 2H), 3.96-4.06 (m, 4H), 3.54 (s, 2H), 3.34 (t, J=12 Hz, 2H), 2.50 (s, 3H), 1.95-1.98 (m, 2H), 1.79-1.82 (m, 2H).

### Example 27

### Preparation of Compound 134—5,7-dichloro-2-(2-(2-methylpyridin-4-yl)-2,6-diazaspiro[3.5]nonan-6-yl)quinoline

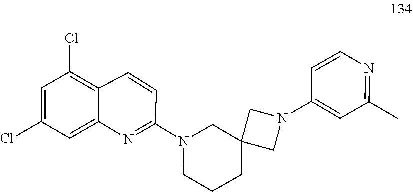 [SMILES: Cc1cc(N2CC3(CCCN(c4ccc5c(Cl)cc(Cl)cc5n4)C3)C2)ccn1]

A mixture of 2-bromo-5,7-dichloroquinoline (30 mg, 0.108 mmol), 2-(2-methylpyridin-4-yl)-2,6-diazaspiro[3.5]nonane (23 mg, 0.108 mmol), and K₂CO₃ (30 mg, 0.216 mmol) in DMF (0.6 mL) was allowed to stir at 140° C. for 4 hours. 5,7-dichloro-2-(2-(2-methylpyridin-4-yl)-2,6-diazaspiro[3.5]nonan-6-yl)quinolone was obtained using prep-TLC. ¹H NMR (400 MHz, CD₃OD) δ: 8.42 (d, 1H), 7.97 (s, 1H), 7.89 (s, 1H), 7.44 (d, 1H), 7.32 (d, 1H), 6.25 (d, 1H), 4.23 (s, 2H), 4.07 (d, 2H), 3.84 (d, 2H), 2.46 (s, 3H), 2.06 (t, 2H), 1.88 (t, 3H).

### Example 28

### Preparation of Compound 135—6-(3-(4-fluoro-3-(trifluoromethyl)phenyl)-1H-pyrazol-5-yl)-2-(2-methylpyridin-4-yl)-2,6-diazaspiro[3.5]nonane

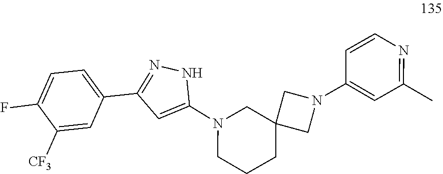 [SMILES: Cc1cc(N2CC3(CCCN(c4cc(-c5ccc(F)c(C(F)(F)F)c5)n[nH]4)C3)C2)ccn1]

### Step 1:

To a solution of 1-(4-fluoro-3-(trifluoromethyl)phenyl)ethan-1-one (0.1 g, 0.5 mmol) in anhydrous DMF (2 mL) was added 30% NaOH (40 mg, 1 mmol) under N₂ atmosphere, then the resulting solution was allowed to stir for 10 minutes. CS₂ (0.04 g, 0.5 mmol), and Me₂SO₄ (0.19 g, 1.5. mmol) were added, and the resulting reaction was allowed to stir overnight. The solution was diluted with 10 mL Et₂O and 10 mL water then separated. The water layer was extracted with Et₂O (10 mL×3). The combined organic layers were washed with brine (10 mL), dried over anhydrous Na₂SO₄, filtered, concentrated and purified using flash chromatography on silica gel (PE/EtOAc: 20/1) to provide 1-(4-fluoro-3-(trifluoromethyl)phenyl)-3,3-bis(methylthio)prop-2-en-1-one, which was used without further purification.

### Step 2:

The solution of 1-(4-fluoro-3-(trifluoromethyl)phenyl)-3,3-bis(methylthio)prop-2-en-1-one (100 mg, 0.36 mmol), and 2-(2-methylpyridin-4-yl)-2,6-diazaspiro[3.5]nonane (80 mg, 0.36 mmol) in anhydrous MeCN (2 mL) was allowed to stir under microwave irradiation for 1 hour at 110° C. Significant product was observed by LCMS, and the solution was used without further purification.

### Step 3:

The solution of 1-(4-fluoro-3-(trifluoromethyl)phenyl)-3-(2-(2-methylpyridin-4-yl)-2,6-diazaspiro[3.5]nonan-6-yl)-3-(methylthio)prop-2-en-1-one (56 mg, 0.1 mmol) from step 2 and NH₂NH₂.H₂O (10 mg, 0.2 mmol) in EtOH (2 mL) was heated at reflux for 3 hours. The reaction mixture was diluted with CH₃OH (2 mL), filtered, and purified using reversed phase PREP-HPLC to provide 6-(3-(4-fluoro-3-(trifluoromethyl)phenyl)-1H-pyrazol-5-yl)-2-(2-methylpyridin-4-yl)-2,6-diazaspiro[3.5]nonane. MS ESI calc'd. for C₂₃H₂₄F₄N₅ [M+H]⁺ 446.1, found 446. ¹H NMR (400 MHz, CD₃OD) δ: 7.90-8.00 (m, 3H), 7.38 (t, J=9.4, 1H), 6.49-6.55 (m, 211), 3.9-4.0 (m, 4H), 3.37 (s, 2H), 3.17-3.19 (m, 2H), 2.5 (s, 3H), 1.89-1.92 (m, 2H), 1.77-1.80 (m, 2H).

### Example 29

### Preparation of Compound 136—6-(3-(3-chloro-5-fluorophenyl)-1H-pyrazol-5-yl)-2-(2-methylpyridin-4-yl)-2,6-diazaspiro[3.5]nonane

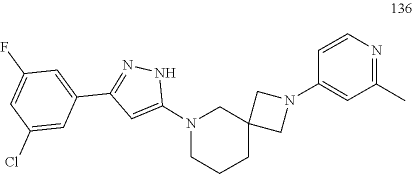 [SMILES: Cc1cc(N2CC3(CCCN(c4cc(-c5cc(F)cc(Cl)c5)n[nH]4)C3)C2)ccn1]

### Step 1:

To a solution of 1-(3-chloro-5-fluorophenyl)ethan-1-one (0.35 g, 2 mmol) in anhydrous DMF (5 mL) was added 30% NaOH (160 mg, 4 mmol) under N₂ atmosphere, and the resulting reaction was allowed to stir at room temperature for 10 minutes. CS₂ (0.15 g, 2 mmol), and Me₂SO₄ (0.76 g, 6. mmol) were added, then the reaction mixture was allowed to stir overnight. The solution was diluted with 30 mL Et₂O and 10 mL water then separated. The water layer was extracted with Et₂O (20 mL×3). The combined organic layers were washed by brine (50 mL), dried over anhydrous Na₂SO₄, filtered, concentrated, and purified using flash chromatography on silica gel (petrol ether/ethyl acetate: 20/1) to provide 1-(3-chloro-5-fluorophenyl)-3,3-bis(methylthio)prop-2-en-1-one, which was used without further purification.

### Step 2:

The solution of 1-(3-chloro-5-fluorophenyl)-3,3-bis(methylthio)prop-2-en-1-one (100 mg, 0.36 mmol), and 2-(2-methylpyridin-4-yl)-2,6-diazaspiro[3.5]nonane (80 mg, 0.36 mmol) in anhydrous MeCN (2 mL) was allowed to stir under microwave irradiation for 1 hour at 110° C. The reaction mixture was confirmed by HPLC to contain 1-(3-chloro-5-fluorophenyl)-3-(2-(2-methylpyridin-4-yl)-2,6-diazaspiro[3.5]nonan-6-yl)-3-(methylthio)prop-2-en-1-one, and the reaction mixture was used without further purification.

### Step 3:

The solution of 1-(3-chloro-5-fluorophenyl)-3-(2-(2-methylpyridin-4-yl)-2,6-diazaspiro[3.5]nonan-6-yl)-3-(methylthio)prop-2-en-1-one (56 mg, 0.1 mmol) from step 2 and NH₂NH₂H₂O (10 mg, 0.2 mmol) in EtOH (2 mL) was heated at reflux for 3 hours. The reaction mixture was diluted with CH₃OH (2 mL), filtered, and purified using reversed phase PREP-HPLC to provide 6-(3-(3-chloro-5-fluorophenyl)-1H-pyrazol-5-yl)-2-(2-methylpyridin-4-yl)-2,6-diazaspiro[3.5]nonane. MS ESI calc'd. for C₂₂H₂₄ClFN₅ [M+H]⁺ 412.1, found 412. ¹H NMR (400 MHz, CD₃OD) δ: 7.96 (d, J=7.0 Hz, 1H), 7.6 (s, 1H), 7.40-7.43 (m, 1H), 7.17-7.19 (m, 1H), 6.53-6.57 (m, 2H), 3.9-4.0 (m, 4H), 3.37 (s, 2H), 3.17-3.19 (m, 2H), 2.5 (s, 3H), 1.89-1.92 (m, 2H), 1.77-1.80 (m, 2H).

### Example 30

### Preparation of Compound 137—6-(4-(3,5-dichlorophenyl)pyridin-2-yl)-2-(2-methylpyridin-4-yl)-2,6-diazaspiro[3.5]nonane

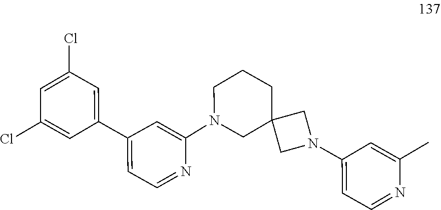 [SMILES: Cc1cc(N2CC3(CCCN(c4cc(-c5cc(Cl)cc(Cl)c5)ccn4)C3)C2)ccn1]

To a stirred solution of 2-chloro-4-(3,5-dichlorophenyl)pyridine (100.0 mg, 0.38 mmol), 2-(2-methylpyridin-4-yl)-2,6-diazaspiro[3.5]nonane (84 mg, 0.38 mmol), t-BuONa (148.2 mg, 1.544 mmol), BINAP (24.0 mg, 0.038 mmol) in toluene (5 mL) was added Pd₂(dba)₃ (35.0 mg, 0.038 mmol) under N₂, and the reaction was heated to 90° C. for 5 hours under N₂ atmosphere. The organic solvent was removed in vacuo and the resulting residue was purified using reversed phase preparative HPLC to provide 6-(4-(3,5-dichlorophenyl)pyridin-2-yl)-2-(2-methylpyridin-4-yl)-2,6-diazaspiro[3.5]nonane. MS ESI calc'd. for C₂₄H₂₅Cl₂N₄ [M+H]⁺ 439.1, found 439. ¹H NMR (400 MHz, CD₃OD) δ: 8.04 (s, 1H), 8.02 (s, 1H), 7.93 (s, 3H), 7.62 (d, 2H), 7.22 (s, 1H), 6.50 (m, 2H), 3.98-4.02 (m, 6H), 3.64-3.70 (s, 2H), 2.45 (s, 3H), 2.04 (s, 2H), 1.85 (s, 2H).

### Example 31

### Preparation of Compound 138—5-(2-(2-methylpyridin-4-yl)-2,6-diazaspiro[3.5]nonan-6-yl)-3-phenyl-1,2,4-oxadiazole

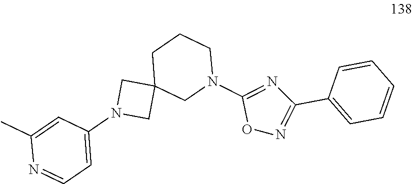 [SMILES: Cc1cc(N2CC3(CCCN(c4nc(-c5ccccc5)no4)C3)C2)ccn1]

### Step 1:

A vial was charged with tert-butyl 2,6-diazaspiro[3.5]nonane-6-carboxylate, Tosic acid (250 mg, 0.627 mmol), 4-bromo-2-methylpyridine (119.1, 1.004 mmol), and 3rd Gen RuPhos precatalyst (52 mg, 0.062 mmol). The vial was sealed and its contents were placed under an inert atmosphere by performing 3 vacuum/nitrogen cycles. THF (4000 μl), and sodium tert-butoxide (627 μl, 1.255 mmol) were added through the septum, and the resulting mixture was allowed to stir overnight at 60° C. The reaction mixture was diluted with Chloroform:Isopropanol and washed twice with Saturated ammonium chloride and once with brine. The combined organic extracts were dried over anhydrous sodium sulfate, filtered, and concentrated in vacuo. The residue was purified using column chromatography on silica (0-10% MeOH:DCM). The desired fractions were concentrated in vacuo to provide tert-butyl 2-(2-methylpyridin-4-yl)-2,6-diazaspiro[3.5]nonane-6-carboxylate. MS ESI calc'd. for C₁₈H₂₇N₃O₂ [M+H]⁺ 318, found 318. 1H NMR (600 MHz, cd3od): δ 7.93 (d, J=7.0 Hz, 1H); 6.49-6.52 (m, 2H); 3.85-3.91 (m, 4H); 3.57 (s, 2H); 3.38 (s, 2H); 2.47 (s, 3H); 1.87 (t, J=5.8 Hz, 2H); 1.55-1.56 (m, 2H); 1.44 (s, 9H).

### Step 2:

4M hydrogen chloride in dioxane (1 ml, 4.00 mmol) was added to a solution of tert-butyl 2-(2-methylpyridin-4-yl)-2,6-diazaspiro[3.5]nonane-6-carboxylate (67 mg, 0.211 mmol) in MeOH (1 mL). The resulting mixture was allowed to stir for 3 hours at 23° C. The reaction mixture was concentrated in vacuo to provide 2-(2-methylpyridin-4-yl)-2,6-diazaspiro[3.5]nonane, 2HCl salt. MS ESI calc'd. for C₁₃H₁₉N₃ [M+H]⁺ 218, found 218. 1H NMR (600 MHz, dmso): δ 13.45 (s, 1H); 9.14 (br d, J=32.6 Hz, 2H); 8.06 (t, J=6.0 Hz, 1H); 6.47-6.48 (m, 2H); 4.06 (t, J=10.0 Hz, 2H); 3.86 (dd, J=19.5, 9.9 Hz, 2H); 3.26 (s, 2H); 2.93 (s, 2H); 2.42 (s, 3H); 1.84 (d, J=6.2 Hz, 2H); 1.68 (s, 2H).

### Step 3:

A solution of 2-(2-methylpyridin-4-yl)-2,6-diazaspiro[3.5]nonane, 2HCl (25 mg, 0.086 mmol) in DMF (500 μl) was added to a vial containing 5-bromo-3-phenyl-1,2,4-oxadiazole (29.1 mg, 0.129 mmol), and potassium carbonate (23.81 mg, 0.172 mmol). The resulting mixture was allowed to stir overnight at 23° C. The reaction mixture was filtered, and purified using mass triggered reverse phase HPLC (ACN/water with 0.1% TFA modifier). The purified fractions were lyophilized via reduced pressure to provide 5-(2-(2-methylpyridin-4-yl)-2,6-diazaspiro[3.5]nonan-6-yl)-3-phenyl-1,2,4-oxadiazole, TFA. MS ESI calc'd. for C₁₃H₁₉N₃ [M+H]⁺ 362, found 362. 1H NMR (600 MHz, dmso): δ 13.18 (s, 1H); 8.08 (s, 1H); 7.86 (d, J=7.5 Hz, 2H); 7.46-7.52 (m, 3H); 6.51-6.52 (m, 2H); 3.85-3.96 (m, 4H); 3.80 (s, 2H); 3.55-3.56 (m, 2H); 2.38 (s, 3H); 1.87 (d, J=6.0 Hz, 2H); 1.63 (s, 2H).

The following compounds were prepared according to the method described in Example 31.

| No. | | [M + H]+ Obs'd |
|---|---|---|
| 139 | 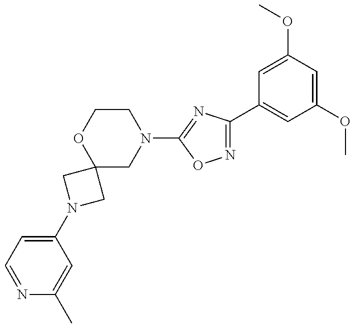 [SMILES: COc1cc(OC)cc(-c2noc(N3CCOC4(CN(c5ccnc(C)c5)C4)C3)n2)c1] | 424 |
| 140 | 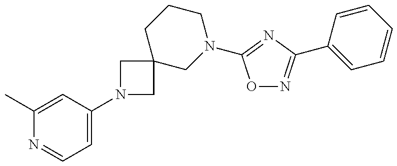 [SMILES: Cc1cc(N2CC3(CCCN(c4nc(-c5ccccc5)no4)C3)C2)ccn1] | 362 |
| 141 | 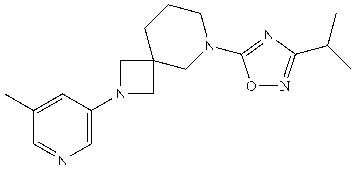 [SMILES: Cc1cncc(N2CC3(CCCN(c4nc(C(C)C)no4)C3)C2)c1] | 328 |
| 142 | 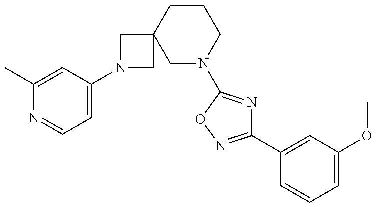 | 392 |
| 143 | 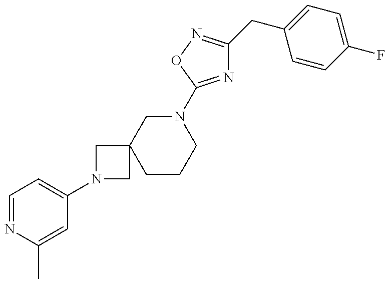 [SMILES: Cc1cc(N2CC3(CCCN(c4nc(Cc5ccc(F)cc5)no4)C3)C2)ccn1] | 394 |
| 144 | 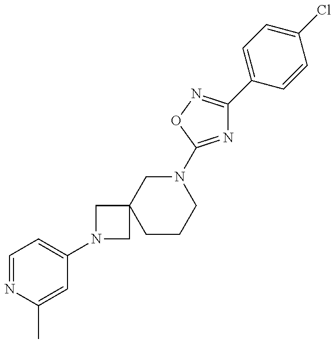 [SMILES: Cc1cc(N2CC3(CCCN(c4nc(-c5ccc(Cl)cc5)no4)C3)C2)ccn1] | 396 |
| 145 | 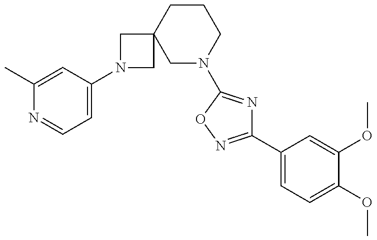 | 422 |
| 146 | 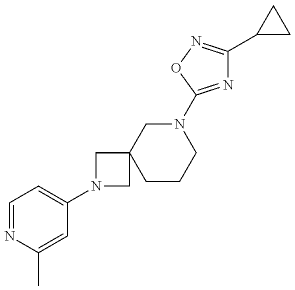 [SMILES: Cc1cc(N2CC3(CCCN(c4nc(C5CC5)no4)C3)C2)ccn1] | 326 |
| 147 | 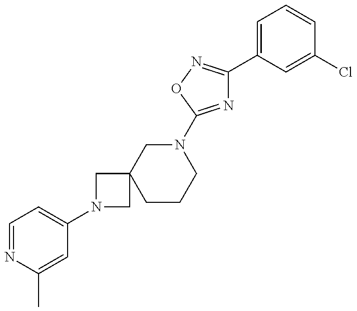 [SMILES: Cc1cc(N2CC3(CCCN(c4nc(-c5cccc(Cl)c5)no4)C3)C2)ccn1] | 396 |
| 148 | 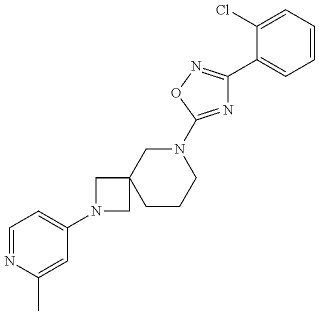 [SMILES: Cc1cc(N2CC3(CCCN(c4nc(-c5ccccc5Cl)no4)C3)C2)ccn1] | 396 |
| 149 | 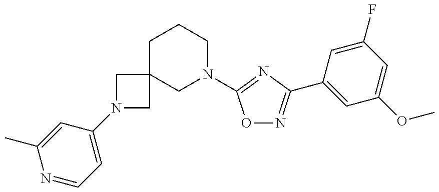 | 410 |
| 150 | 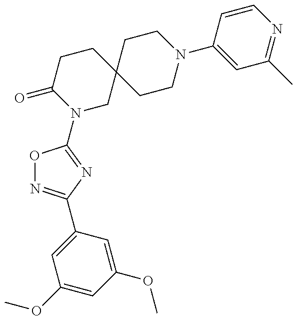 | 464 |
| 151 | 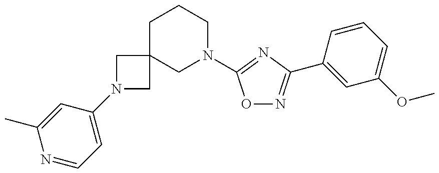 | 392 |
| 152 | 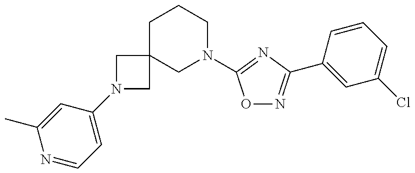 [SMILES: Cc1cc(N2CC3(CCCN(c4nc(-c5cccc(Cl)c5)no4)C3)C2)ccn1] | 396 |

### Example 32

### Preparation of Compound 153—2-(3-(3-methoxyphenyl)-1,2,4-oxadiazol-5-yl)-9-(2-methylpyridin-4-yl)-2,9-diazaspiro[5.5]undecan-3-one, TFA salt

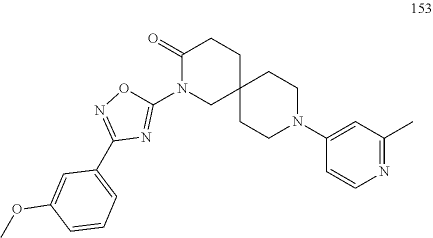

### Step 1:

A solution of 4-fluoro-2-methylpyridine (64.1 μL, 0.622 mmol) in DMSO (250 μL) was added to a vial containing 2,9-diazaspiro[5.5]undecan-3-one, 2HCl (75 mg, 0.311 mmol). DIEA (250 μL, 1.431 mmol) was added, and the resulting mixture was allowed to stir for 1.5 hours at 160° C. The reaction mixture was diluted with EtOAc and washed twice with water and once with brine. The combined organic extracts were dried over anhydrous sodium sulfate, filtered, and concentrated in vacuo to provide 9-(2-methylpyridin-4-yl)-2,9-diazaspiro[5.5]undecan-3-one. MS ESI calc'd. for C₁₅H₂₁N₃₀ [M+H]⁺ 260, found 260.

### Step 2:

A vial was charged with 9-(2-methylpyridin-4-yl)-2,9-diazaspiro[5.5]undecan-3-one (45 mg, 0.174 mmol), 5-chloro-3-(3-methoxyphenyl)-1,2,4-oxadiazole (45 mg, 0.214 mmol), 3rd Gen BrettPhos Pre-Catalyst (31.4 mg, 0.035 mmol), and cesium carbonate (113 mg, 0.347 mmol). The vial was sealed and its contents were placed under an inert atmosphere by performing 3 vacuum/nitrogen purge cycles. THF (1000 μl) was added through the septum and the resulting mixture was allowed to stir overnight at 80° C. The reaction mixture was filtered, and submitted directly for HPLC purification (using HPLC, eluting acetonitrile/water gradient with 0.1% TFA modifier, linear gradient), and lyophilized to provide 2-(3-(3-methoxyphenyl)-1,2,4-oxadiazol-5-yl)-9-(2-methylpyridin-4-yl)-2,9-diazaspiro[5.5]undecan-3-one, TFA. MS ESI calc'd. for C₂₄H₂₇N₅O₃ TFA [M+H]⁺ 434, found 434.

### Example 33

### Preparation of Compound 154—6-(3-(3,5-dichlorophenyl)-1,2,4-oxadiazol-5-yl)-N-(2-methylpyridin-4-yl)-6-azaspiro[3.4]octan-2-amine, TFA salt

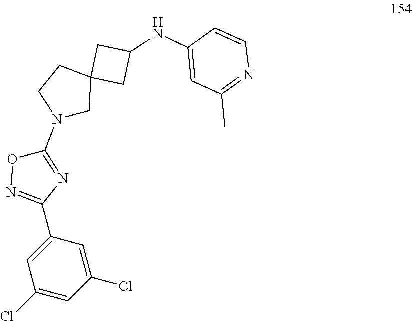 [SMILES: Cc1cc(NC2CC3(CCN(c4nc(-c5cc(Cl)cc(Cl)c5)no4)C3)C2)ccn1]

### Step 1:

A solution of 5-chloro-3-(3,5-dichlorophenyl)-1,2,4-oxadiazole (40 mg, 0.160 mmol) in THF (1 mL) was added to a vial containing tert-butyl 6-azaspiro[3.4]octan-2-ylcarbamate (43.5 mg, 0.192 mmol). DIEA (100 μl, 0.573 mmol) was added, and the resulting mixture was allowed to stir overnight at room temperature. MS ESI calc'd. for C₂₀H₂₄Cl₂N₄O₃ [M+H]⁺ 439, found 439.

### Step 2:

TFA (1 ml, 12.98 mmol) was added to tert-butyl (6-(3-(3,5-dichlorophenyl)-1,2,4-oxadiazol-5-yl)-6-azaspiro[3.4]octan-2-yl)carbamate (0.070 g, 0.16 mmol). The resulting mixture was allowed to stir for 1 hour at room temperature. The reaction mixture was concentrated in vacuo. MS ESI calc'd. for C₁₅H₁₆Cl₂N₄O [M+H]⁺ 339, found 339.

### Step 3:

A solution of 4-fluoro-2-methylpyridine (35.6 mg, 0.320 mmol) in NMP (1 mL) was added to a vial containing 6-(3-(3,5-dichlorophenyl)-1,2,4-oxadiazol-5-yl)-6-azaspiro[3.4]octan-2-amine (54.3 mg, 0.16 mmol). DIEA (0.2 ml, 1.145 mmol) was added, and the resulting mixture was allowed to stir for 2 hours at 160° C. The reaction mixture was filtered, and submitted directly for HPLC purification to the HTP group (purified using HPLC, eluting acetonitrile/water gradient with 0.1% TFA modifier, linear gradient), and lyophilized to provide 6-(3-(3,5-dichlorophenyl)-1,2,4-oxadiazol-5-yl)-N-(2-methylpyridin-4-yl)-6-azaspiro[3.4]octan-2-amine, TFA. MS ESI calc'd. for C₁₅H₁₆Cl₂N₄O [M+H]⁺ 430, found 430. 1H NMR (600 MHz, DMSO-d6) δ 13.24-13.05 (m, 1H), 8.74-8.69 (m, 1H), 7.84-7.78 (m, 3H), 6.72-6.63 (m, 2H), 4.26-4.07 (m, 1H), 3.67 (s, 1H), 3.62-3.58 (m, 1H), 3.56-3.51 (m, 2H), 2.55-2.49 (m, 2H), 2.43-2.40 (m, 2H), 2.37 (s, 1H), 2.10 (t, J=6.2 Hz, 1H), 2.08-2.03 (m, 1H), 2.03-1.96 (m, 3H).

The following compounds were prepared according to the method described in Example 33.

| | | [M + H] + |
|---|---|---|
| No. | | Obs′d |
| 155 | 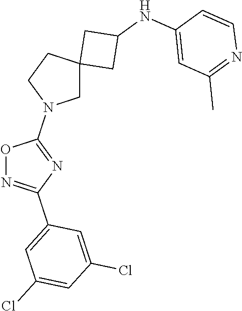 [SMILES: Cc1cc(NC2CC3(CCN(c4nc(-c5cc(Cl)cc(Cl)c5)no4)C3)C2)ccn1] | 430 |
| 156 | 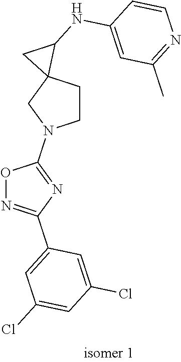 [SMILES: Cc1cc(NC2CC23CCN(c2nc(-c4cc(Cl)cc(Cl)c4)no2)C3)ccn1] | 416 |
| 157 | 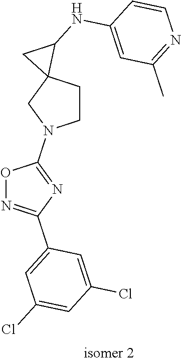 [SMILES: Cc1cc(NC2CC23CCN(c2nc(-c4cc(Cl)cc(Cl)c4)no2)C3)ccn1] | 416 |
| 158 | 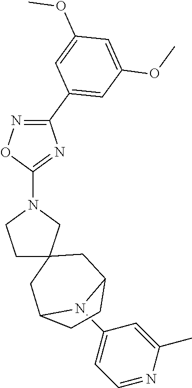 [SMILES: COc1cc(OC)cc(-c2noc(N3CCC4(CC5CCC(C4)N5c4ccnc(C)c4)C3)n2)c1] | 462 |
| 159 | 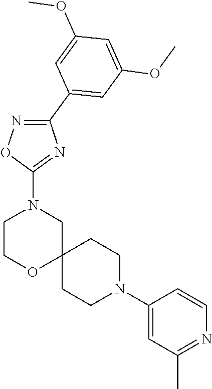 [SMILES: COc1cc(OC)cc(-c2noc(N3CCOC4(CCN(c5ccnc(C)c5)CC4)C3)n2)c1] | 451 |
| 160 | 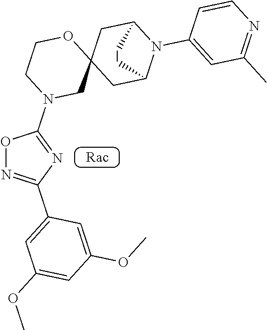 [SMILES: COc1cc(OC)cc(-c2noc(N3CCO[C@]4(C[C@H]5CC[C@@H](C4)N5c4ccnc(C)c4)C3)n2)c1] | 478 |
| 161 | 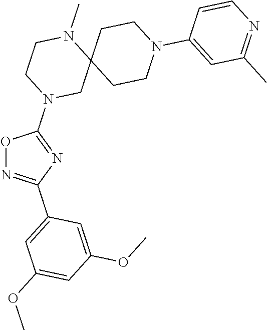 [SMILES: COc1cc(OC)cc(-c2noc(N3CCN(C)C4(CCN(c5ccnc(C)c5)CC4)C3)n2)c1] | 465 |
| 162 | 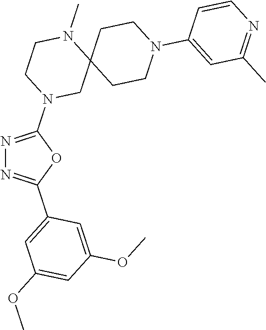 [SMILES: COc1cc(OC)cc(-c2nnc(N3CCN(C)C4(CCN(c5ccnc(C)c5)CC4)C3)o2)c1] | 465 |
| 163 | 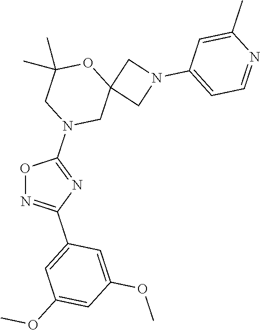 [SMILES: COc1cc(OC)cc(-c2noc(N3CC(C)(C)OC4(CN(c5ccnc(C)c5)C4)C3)n2)c1] | 452 |
| 164 | 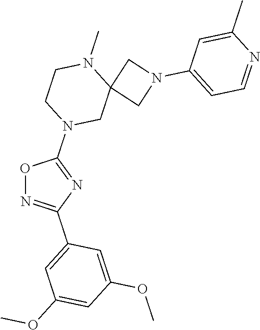 [SMILES: COc1cc(OC)cc(-c2noc(N3CCN(C)C4(CN(c5ccnc(C)c5)C4)C3)n2)c1] | 437 |
| 165 | 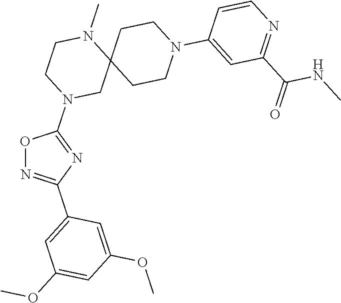 | 508 |
| 166 | 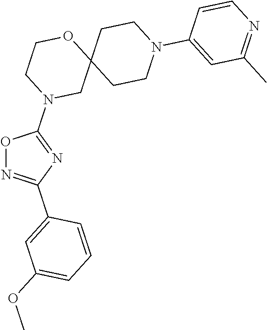 [SMILES: COc1cccc(-c2noc(N3CCOC4(CCN(c5ccnc(C)c5)CC4)C3)n2)c1] | 422 |
| 167 | 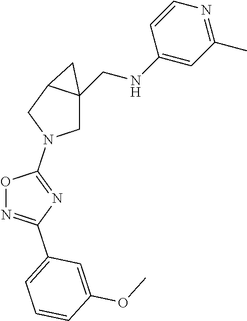 [SMILES: COc1cccc(-c2noc(N3CC4CC4(CNc4ccnc(C)c4)C3)n2)c1] | 378 |
| 168 | 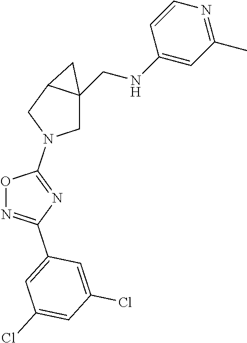 [SMILES: Cc1cc(NCC23CC2CN(c2nc(-c4cc(Cl)cc(Cl)c4)no2)C3)ccn1] | 416 |
| 169 | 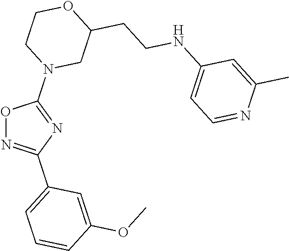 [SMILES: COc1cccc(-c2noc(N3CCOC(CCNc4ccnc(C)c4)C3)n2)c1] | 396 |
| 170 | 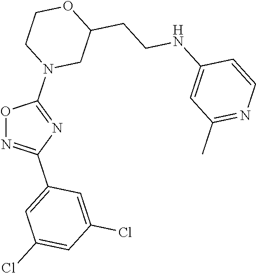 [SMILES: Cc1cc(NCCC2CN(c3nc(-c4cc(Cl)cc(Cl)c4)no3)CCO2)ccn1] | 434 |
| 171 | 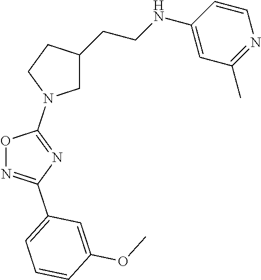 [SMILES: COc1cccc(-c2noc(N3CCC(CCNc4ccnc(C)c4)C3)n2)c1] | 380 |
| 172 | 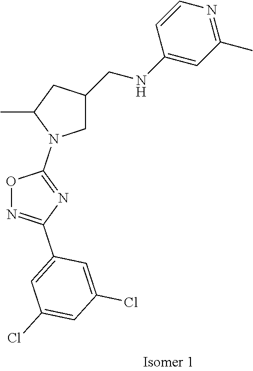 | 418 |
| 173 | 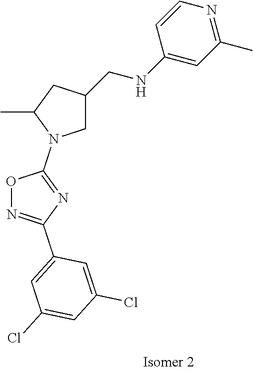 | 418 |
| 174 | 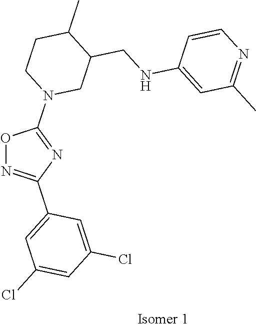 [SMILES: Cc1cc(NCC2CN(c3nc(-c4cc(Cl)cc(Cl)c4)no3)CCC2C)ccn1] | 432 |
| 175 | 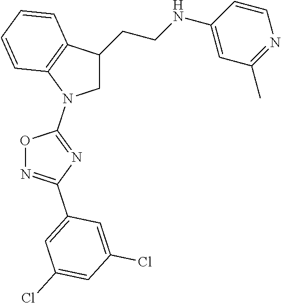 [SMILES: Cc1cc(NCCC2CN(c3nc(-c4cc(Cl)cc(Cl)c4)no3)c3ccccc32)ccn1] | 466 |
| 176 | 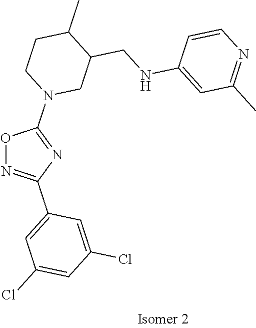 [SMILES: Cc1cc(NCC2CN(c3nc(-c4cc(Cl)cc(Cl)c4)no3)CCC2C)ccn1] | 432 |
| 177 | 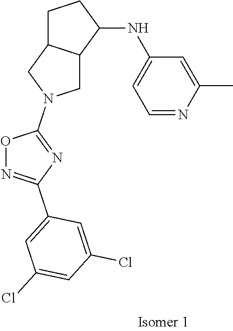 [SMILES: Cc1cc(NC2CCC3CN(c4nc(-c5cc(Cl)cc(Cl)c5)no4)CC32)ccn1] | 430 |
| 178 | 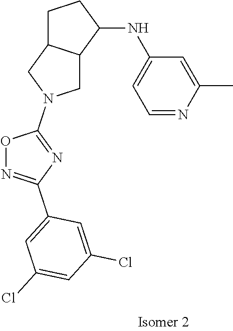 [SMILES: Cc1cc(NC2CCC3CN(c4nc(-c5cc(Cl)cc(Cl)c5)no4)CC32)ccn1] | 430 |
| 179 | 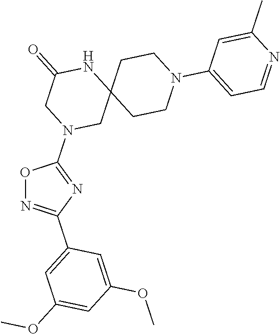 [SMILES: COc1cc(OC)cc(-c2noc(N3CC(=O)NC4(CCN(c5ccnc(C)c5)CC4)C3)n2)c1] | 465 |
| 180 | 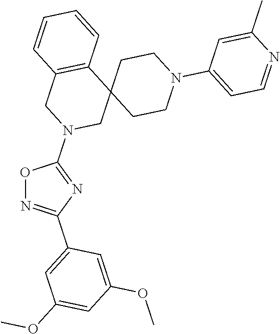 [SMILES: COc1cc(OC)cc(-c2noc(N3Cc4ccccc4C4(CCN(c5ccnc(C)c5)CC4)C3)n2)c1] | 498 |
| 181 | 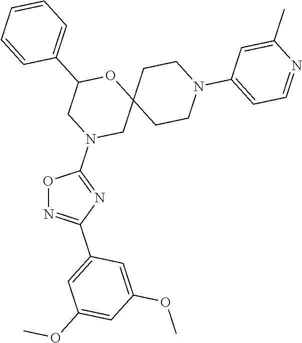 [SMILES: COc1cc(OC)cc(-c2noc(N3CC(c4ccccc4)OC4(CCN(c5ccnc(C)c5)CC4)C3)n2)c1] | 528 |
| 182 | 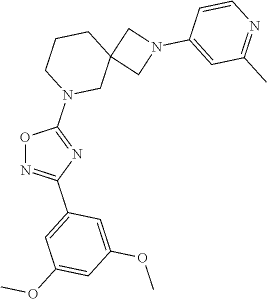 | 422 |
| 183 | 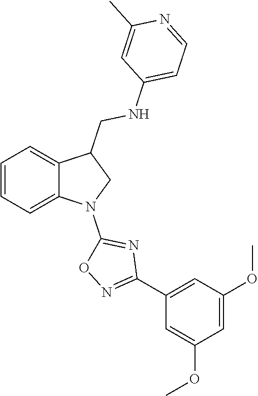 [SMILES: COc1cc(OC)cc(-c2noc(N3CC(CNc4ccnc(C)c4)c4ccccc43)n2)c1] | 444 |
| 184 | 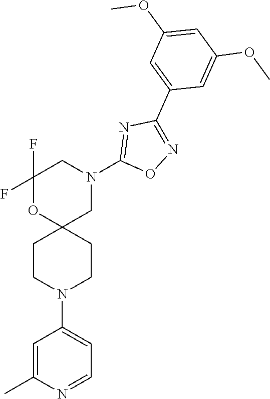 | 488 |
| 185 | 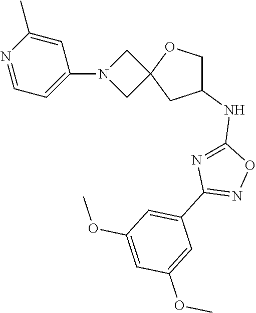 [SMILES: COc1cc(OC)cc(-c2noc(NC3COC4(C3)CN(c3ccnc(C)c3)C4)n2)c1] | 424 |
| 186 | 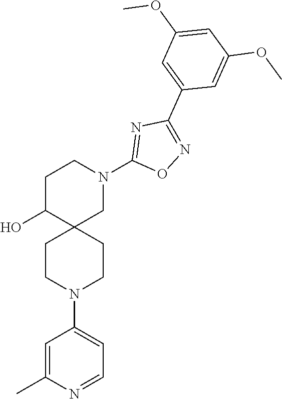 | 466 |
| 187 | 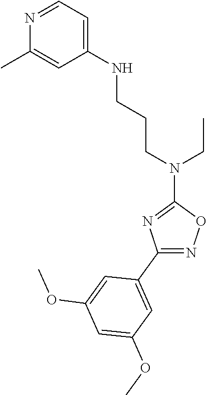 [SMILES: CCN(CCCNc1ccnc(C)c1)c1nc(-c2cc(OC)cc(OC)c2)no1] | 398 |
| 188 | 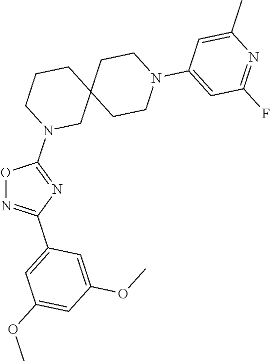 [SMILES: COc1cc(OC)cc(-c2noc(N3CCCC4(CCN(c5cc(C)nc(F)c5)CC4)C3)n2)c1] | 468 |

### Example 34

### Preparation of Compound 189—3-(3,5-dichlorophenyl)-5-(2-(2-methylpyridin-4-yl)-2,6-diazaspiro[3.5]nonan-6-yl)-1,2,4-oxadiazole

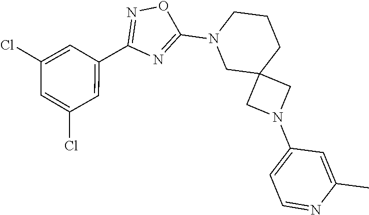 [SMILES: Cc1cc(N2CC3(CCCN(c4nc(-c5cc(Cl)cc(Cl)c5)no4)C3)C2)ccn1]

### Step 1:

A solution of tert-butyl 2,6-diazaspiro[3.5]nonane-2-carboxylate, oxalic acid (114 mg, 0.360 mmol) in DMF (3000 μl) was added to a vial containing potassium carbonate (83 mg, 0.600 mmol), and 5-chloro-3-(3,5-dichlorophenyl)-1,2,4-oxadiazole (74.8 mg, 0.3 mmol). The resulting mixture was allowed to stir for 8 hours at 23° C. at which point a precipitate had formed. Water was added to the precipitate and the solids were collected by vacuum filtration and dried to provide tert-butyl 6-(3-(3,5-dichlorophenyl)-1,2,4-oxadiazol-5-yl)-2,6-diazaspiro[3.5]nonane-2-carboxylate. MS ESI calc'd. for C₂₀H₂₄Cl₂N₄O₃ [M+H]⁺ 439, found 383. ¹H NMR (600 MHz, dmso): δ 7.81-7.81 (m, 3H); 3.68 (br s, 2H); 3.52 (br s, 6H); 1.75 (s, 2H); 1.57 (s, 2H); 1.34 (s, 9H).

### Step 2:

4M HCl in dioxane (1.5 ml, 6.00 mmol) was added to a solution of tert-butyl 6-(3-(3,5-dichlorophenyl)-1,2,4-oxadiazol-5-yl)-2,6-diazaspiro[3.5]nonane-2-carboxylate (135 mg, 0.307 mmol) in MeOH (4 mL). The resulting mixture was allowed to stir for 3 hours at 23° C. The reaction mixture was concentrated in vacuo to provide 3-(3,5-dichlorophenyl)-5-(2,6-diazaspiro[3.5]nonan-6-yl)-1,2,4-oxadiazole, 2HCl salt. MS ESI calc'd. for C₁₅H₁₆Cl₂N₄O [M+H]⁺ 339, found 339. ¹H NMR (600 MHz, dmso): δ 9.00 (br s, 1H); 8.85 (br s, 1H); 7.82 (s, 3H); 3.80 (s, 2H); 3.63-3.68 (br m, 4H); 3.49 (t, J=5.6 Hz, 2H); 1.82 (t, J=5.7 Hz, 2H); 1.58 (d, J=6.6 Hz, 2H).

### Step 3:

A microwave vial was charged with 3-(3,5-dichlorophenyl)-5-(2,6-diazaspiro[3.5]nonan-6-yl)-1,2,4-oxadiazole, 2HCl (41.2 mg, 0.1 mmol), and 3rd Gen RuPhos Precatalyst (12.55 mg, 0.015 mmol). The vial was sealed and its contents were placed under an inert atmosphere by performing 3 vacuum/nitrogen cycles. A solution of 4-bromo-2-methylpyridine (0.018 ml, 0.150 mmol) in THF (500 mL), and sodium tert-butoxide (0.200 ml, 0.200 mmol) was added through the septum. The resulting mixture was allowed to stir overnight at 60° C. The reaction mixture was dissolved with MeOH, and concentrated in vacuo. The crude product was dissolved in DMSO, filtered, and purified using mass triggered reverse phase HPLC (ACN/water with 0.1% TFA modifier). The purified fractions were lyophilized via reduced pressure to provide 3-(3,5-dichlorophenyl)-5-(2-(2-methylpyridin-4-yl)-2,6-diazaspiro[3.5]nonan-6-yl)-1,2,4-oxadiazole, TFA. MS ESI calc'd. for C₂₁H₂₁Cl₂N₅O [M+H]⁺ 430, found 430. ¹H NMR (600 MHz, dmso): δ 13.13 (s, 1H); 8.07-8.08 (m, 1H); 7.80-7.81 (m, 3H); 6.51-6.52 (m, 2H); 3.94 (dd, J=16.7, 9.8 Hz, 2H); 3.87 (dd, J=17.2, 9.8 Hz, 2H); 3.81 (s, 2H); 3.55-3.56 (m, 2H); 2.38 (s, 3H); 1.87-1.88 (m, 2H); 1.63 (s, 2H).

The following compounds were prepared according to the method described in Example 34.

| No. | | [M + 11] + Obs′d |
|---|---|---|
| 190 | 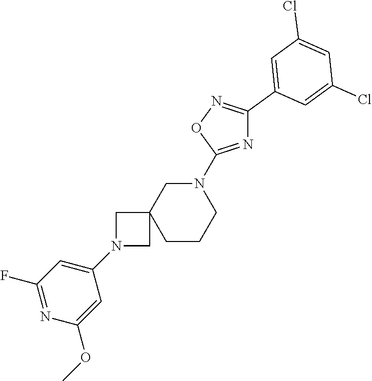 [SMILES: COc1cc(N2CC3(CCCN(c4nc(-c5cc(Cl)cc(Cl)c5)no4)C3)C2)cc(F)n1] | 464 |
| 191 | 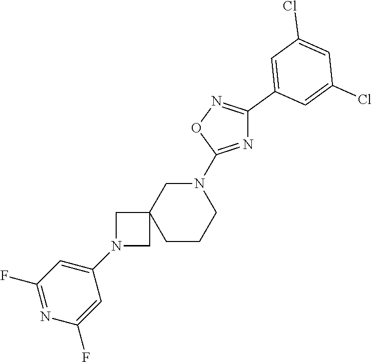 [SMILES: Fc1cc(N2CC3(CCCN(c4nc(-c5cc(Cl)cc(Cl)c5)no4)C3)C2)cc(F)n1] | 452 |
| 192 | 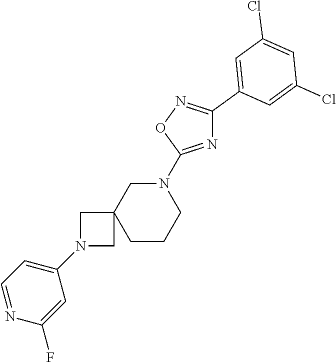 [SMILES: Fc1cc(N2CC3(CCCN(c4nc(-c5cc(Cl)cc(Cl)c5)no4)C3)C2)ccn1] | 434 |
| 193 | 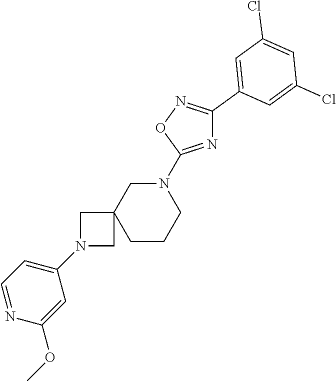 [SMILES: COc1cc(N2CC3(CCCN(c4nc(-c5cc(Cl)cc(Cl)c5)no4)C3)C2)ccn1] | 446 |
| 194 | 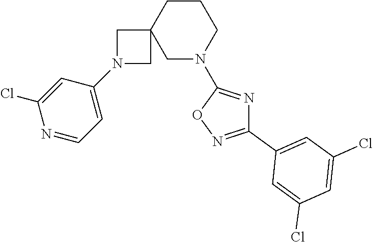 [SMILES: Clc1cc(Cl)cc(-c2noc(N3CCCC4(CN(c5ccnc(Cl)c5)C4)C3)n2)c1] | 450 |
| 195 | 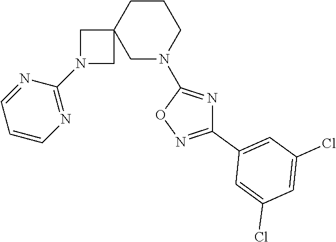 [SMILES: Clc1cc(Cl)cc(-c2noc(N3CCCC4(CN(c5ncccn5)C4)C3)n2)c1] | 417 |
| 196 | 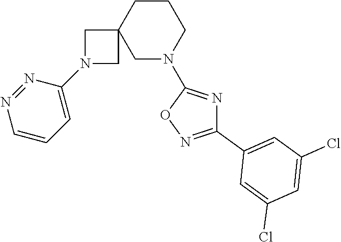 [SMILES: Clc1cc(Cl)cc(-c2noc(N3CCCC4(CN(c5cccnn5)C4)C3)n2)c1] | 417 |
| 197 | 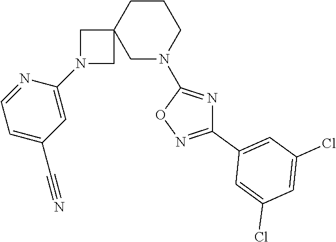 [SMILES: N#Cc1ccnc(N2CC3(CCCN(c4nc(-c5cc(Cl)cc(Cl)c5)no4)C3)C2)c1] | 441 |
| 198 | 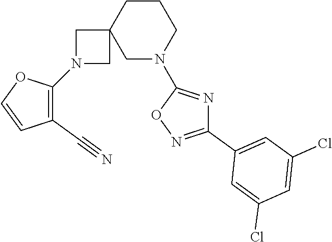 [SMILES: N#Cc1ccoc1N1CC2(CCCN(c3nc(-c4cc(Cl)cc(Cl)c4)no3)C2)C1] | 430 |
| 199 | 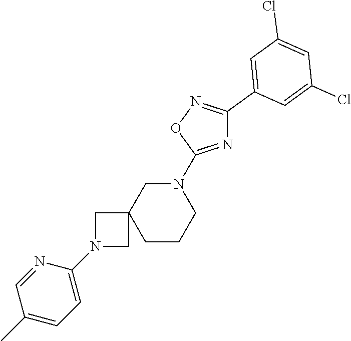 [SMILES: Cc1ccc(N2CC3(CCCN(c4nc(-c5cc(Cl)cc(Cl)c5)no4)C3)C2)nc1] | 430 |
| 200 | 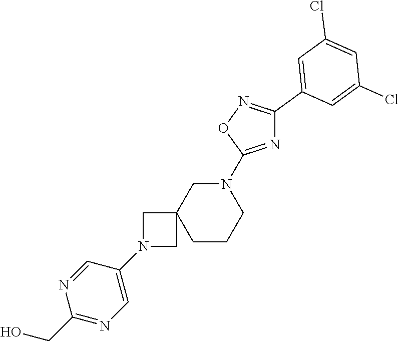 [SMILES: OCc1ncc(N2CC3(CCCN(c4nc(-c5cc(Cl)cc(Cl)c5)no4)C3)C2)cn1] | 447 |
| 201 | 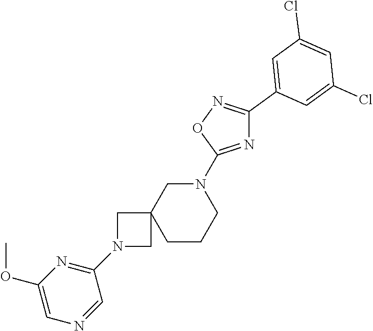 [SMILES: COc1cncc(N2CC3(CCCN(c4nc(-c5cc(Cl)cc(Cl)c5)no4)C3)C2)n1] | 447 |
| 202 | 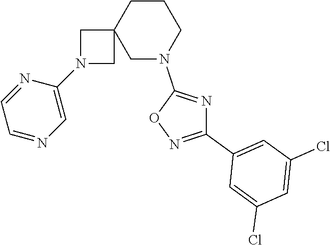 [SMILES: Clc1cc(Cl)cc(-c2noc(N3CCCC4(CN(c5cnccn5)C4)C3)n2)c1] | 417 |
| 203 | 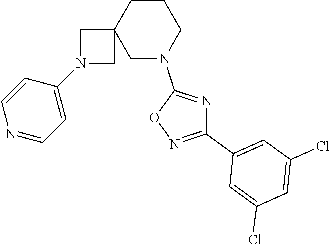 [SMILES: Clc1cc(Cl)cc(-c2noc(N3CCCC4(CN(c5ccncc5)C4)C3)n2)c1] | 416 |
| 204 | 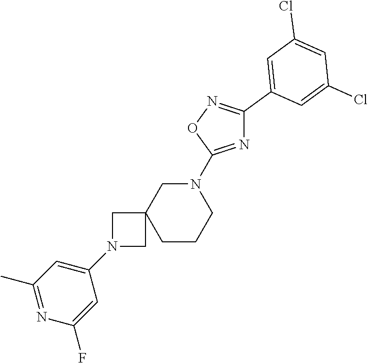 [SMILES: Cc1cc(N2CC3(CCCN(c4nc(-c5cc(Cl)cc(Cl)c5)no4)C3)C2)cc(F)n1] | 448 |
| 205 | 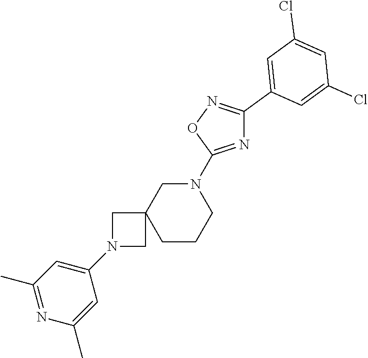 [SMILES: Cc1cc(N2CC3(CCCN(c4nc(-c5cc(Cl)cc(Cl)c5)no4)C3)C2)cc(C)n1] | 444 |
| 206 | 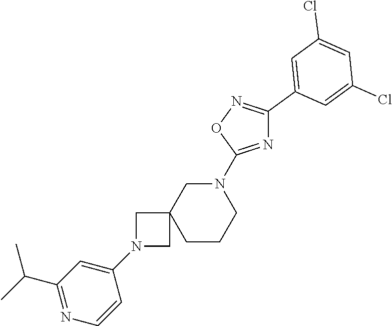 [SMILES: CC(C)c1cc(N2CC3(CCCN(c4nc(-c5cc(Cl)cc(Cl)c5)no4)C3)C2)ccn1] | 458 |
| 207 | 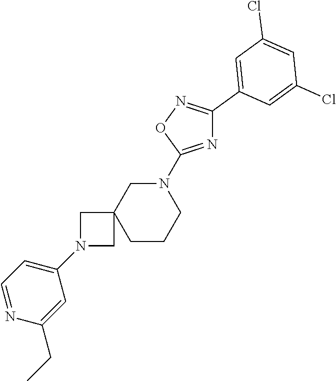 [SMILES: CCc1cc(N2CC3(CCCN(c4nc(-c5cc(Cl)cc(Cl)c5)no4)C3)C2)ccn1] | 444 |
| 208 | 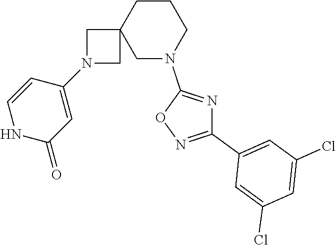 [SMILES: O=c1cc(N2CC3(CCCN(c4nc(-c5cc(Cl)cc(Cl)c5)no4)C3)C2)cc[nH]1] | 432 |
| 209 | 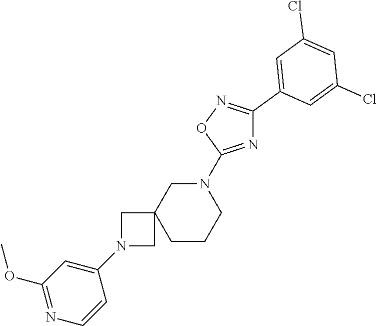 [SMILES: COc1cc(N2CC3(CCCN(c4nc(-c5cc(Cl)cc(Cl)c5)no4)C3)C2)ccn1] | 446 |
| 210 | 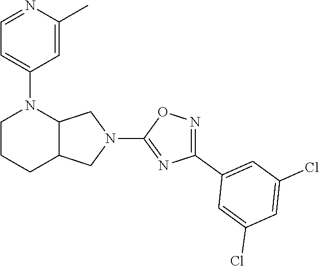 [SMILES: Cc1cc(N2CCCC3CN(c4nc(-c5cc(Cl)cc(Cl)c5)no4)CC32)ccn1] | 430 |
| 211 | 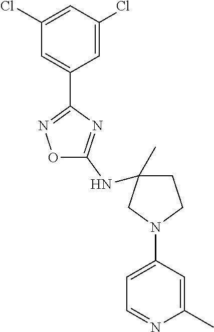 [SMILES: Cc1cc(N2CCC(C)(Nc3nc(-c4cc(Cl)cc(Cl)c4)no3)C2)ccn1] | 404 |
| 212 | 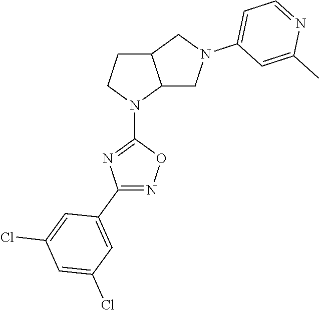 [SMILES: Cc1cc(N2CC3CCN(c4nc(-c5cc(Cl)cc(Cl)c5)no4)C3C2)ccn1] | 416 |
| 213 | 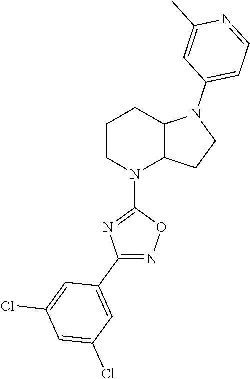 [SMILES: Cc1cc(N2CCC3C2CCCN3c2nc(-c3cc(Cl)cc(Cl)c3)no2)ccn1] | 430 |
| 214 | 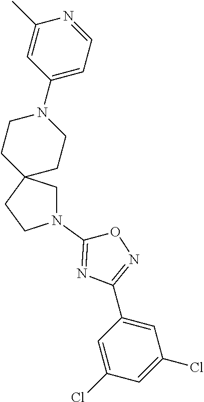 [SMILES: Cc1cc(N2CCC3(CC2)CCN(c2nc(-c4cc(Cl)cc(Cl)c4)no2)C3)ccn1] | 444 |
| 215 | 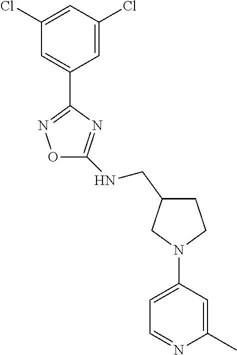 [SMILES: Cc1cc(N2CCC(CNc3nc(-c4cc(Cl)cc(Cl)c4)no3)C2)ccn1] | 404 |
| 216 | 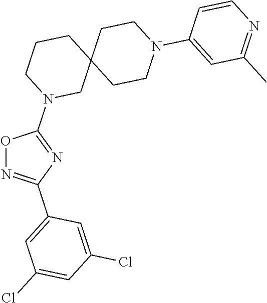 [SMILES: Cc1cc(N2CCC3(CCCN(c4nc(-c5cc(Cl)cc(Cl)c5)no4)C3)CC2)ccn1] | 458 |
| 217 | 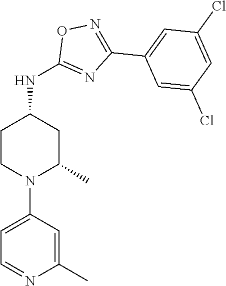 [SMILES: Cc1cc(N2CC[C@H](Nc3nc(-c4cc(Cl)cc(Cl)c4)no3)C[C@@H]2C)ccn1] | 418 |
| 218 | 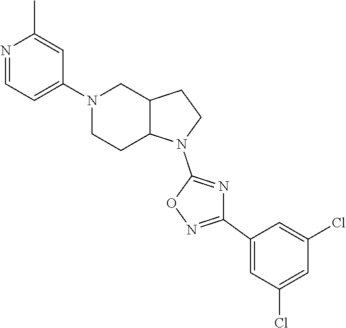 [SMILES: Cc1cc(N2CCC3C(CCN3c3nc(-c4cc(Cl)cc(Cl)c4)no3)C2)ccn1] | 430 |
| 219 | 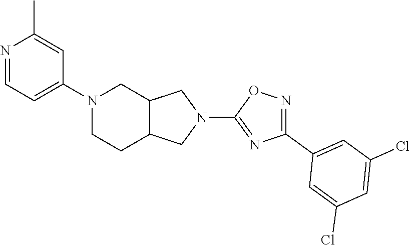 [SMILES: Cc1cc(N2CCC3CN(c4nc(-c5cc(Cl)cc(Cl)c5)no4)CC3C2)ccn1] | 430 |
| 220 | 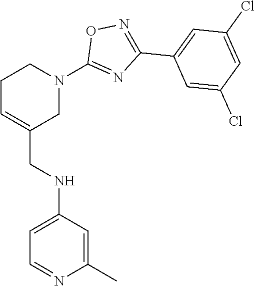 [SMILES: Cc1cc(NCC2=CCCN(c3nc(-c4cc(Cl)cc(Cl)c4)no3)C2)ccn1] | 416 |
| 221 | 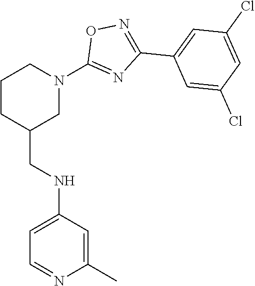 [SMILES: Cc1cc(NCC2CCCN(c3nc(-c4cc(Cl)cc(Cl)c4)no3)C2)ccn1] | 418 |
| 222 | 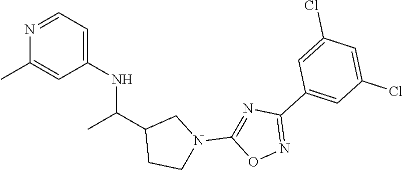 [SMILES: Cc1cc(NC(C)C2CCN(c3nc(-c4cc(Cl)cc(Cl)c4)no3)C2)ccn1] | 418 |
| 223 | 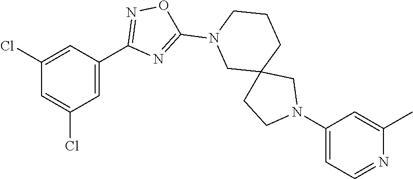 [SMILES: Cc1cc(N2CCC3(CCCN(c4nc(-c5cc(Cl)cc(Cl)c5)no4)C3)C2)ccn1] | 444 |
| 224 | 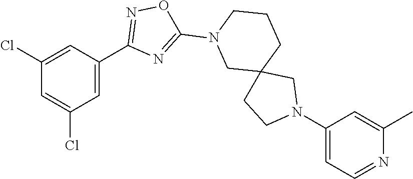 [SMILES: Cc1cc(N2CCC3(CCCN(c4nc(-c5cc(Cl)cc(Cl)c5)no4)C3)C2)ccn1] | 444 |
| 225 | 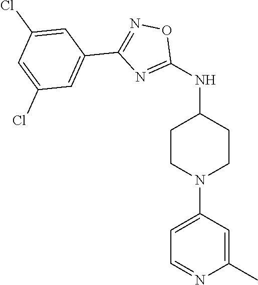 [SMILES: Cc1cc(N2CCC(Nc3nc(-c4cc(Cl)cc(Cl)c4)no3)CC2)ccn1] | 404 |
| 226 | 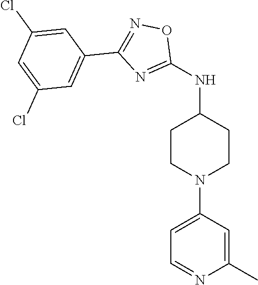 [SMILES: Cc1cc(N2CCC(Nc3nc(-c4cc(Cl)cc(Cl)c4)no3)CC2)ccn1] | 404 |
| 227 | 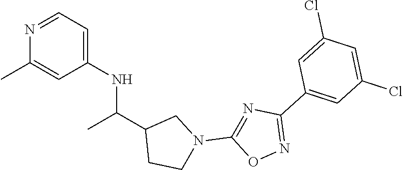 [SMILES: Cc1cc(NC(C)C2CCN(c3nc(-c4cc(Cl)cc(Cl)c4)no3)C2)ccn1] | 418 |
| 228 | 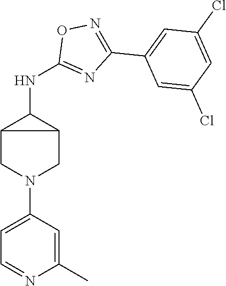 [SMILES: Cc1cc(N2CC3C(C2)C3Nc2nc(-c3cc(Cl)cc(Cl)c3)no2)ccn1] | 402 |
| 229 | 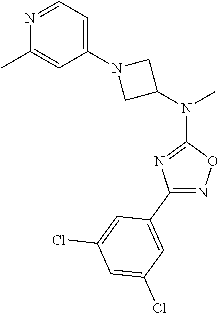 | 390 |
| 230 | 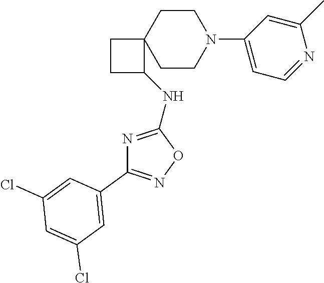 [SMILES: Cc1cc(N2CCC3(CCC3Nc3nc(-c4cc(Cl)cc(Cl)c4)no3)CC2)ccn1] | 444 |
| 231 | 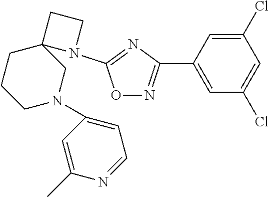 [SMILES: Cc1cc(N2CCCC3(CCN3c3nc(-c4cc(Cl)cc(Cl)c4)no3)C2)ccn1] | 430 |
| 232 | 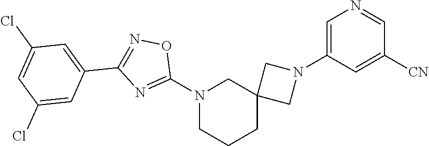 [SMILES: N#Cc1cncc(N2CC3(CCCN(c4nc(-c5cc(Cl)cc(Cl)c5)no4)C3)C2)c1] | 441 |
| 233 | 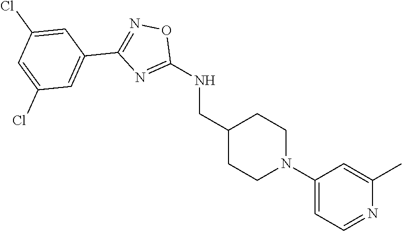 [SMILES: Cc1cc(N2CCC(CNc3nc(-c4cc(Cl)cc(Cl)c4)no3)CC2)ccn1] | 418 |
| 234 | 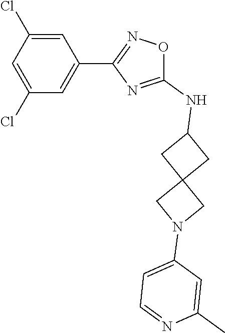 [SMILES: Cc1cc(N2CC3(CC(Nc4nc(-c5cc(Cl)cc(Cl)c5)no4)C3)C2)ccn1] | 416 |
| 235 | 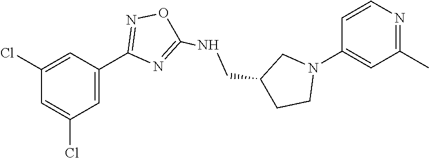 [SMILES: Cc1cc(N2CC[C@H](CNc3nc(-c4cc(Cl)cc(Cl)c4)no3)C2)ccn1] | 404 |
| 236 | 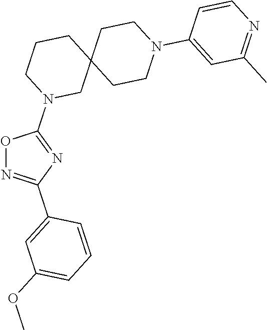 [SMILES: COc1cccc(-c2noc(N3CCCC4(CCN(c5ccnc(C)c5)CC4)C3)n2)c1] | 420 |
| 237 | 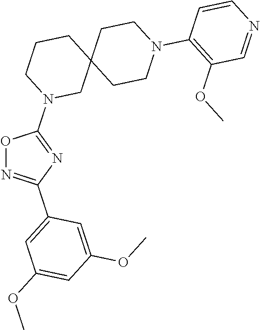 [SMILES: COc1cc(OC)cc(-c2noc(N3CCCC4(CCN(c5ccncc5OC)CC4)C3)n2)c1] | 466 |

### Example 35

### Preparation of Compound 238—1-(3-(3,5-dichlorophenyl)-1,2,4-oxadiazol-5-yl)-N-(2-methylpyridin-4-yl)piperidine-4-carboxamide

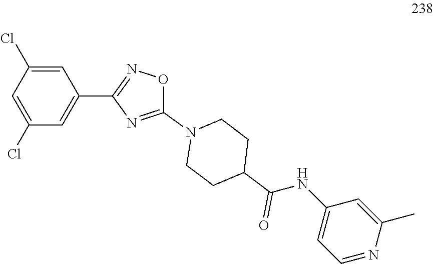 [SMILES: Cc1cc(NC(=O)C2CCN(c3nc(-c4cc(Cl)cc(Cl)c4)no3)CC2)ccn1]

### Step 1:

A solution of 5-chloro-3-(3,5-dichlorophenyl)-1,2,4-oxadiazole (0.15 g, 0.601 mmol) in DMF (3.01 mL) was added to a vial containing piperidine-4-carboxylic acid (0.100 g, 0.774 mmol). DIEA (0.500 ml, 2.86 mmol) was added, and the resulting mixture was allowed to stir over the weekend at room temperature. The reaction mixture was concentrated in vacuo. MS ESI calc'd. for C₁₄H₁₃Cl₂N₃O₃ [M+H]⁺ 342, found 342.

### Step 2:

A solution of 1-(3-(3,5-dichlorophenyl)-1,2,4-oxadiazol-5-yl)piperidine-4-carboxylic acid (50 mg, 0.146 mmol) in DMF (1 mL) was added to vial containing 2-methylpyridin-4-amine (23.70 mg, 0.219 mmol), and HATU (83 mg, 0.219 mmol). DIEA (0.051 ml, 0.292 mmol) was added, and the resulting mixture was allowed to stir overnight at room temperature. The reaction mixture was filtered, and purified using mass triggered reverse phase HPLC (ACN/water with 0.1% TFA modifier). The purified fraction was lyophilized via reduced pressure to provide 1-(3-(3,5-dichlorophenyl)-1,2,4-oxadiazol-5-yl)-N-(2-methylpyridin-4-yl)piperidine-4-carboxamide. MS ESI calc'd. for C₂₀H₁₉Cl₂N₅O₂ [M+H]⁺ 432, found 432. 1H NMR (600 MHz, dmso): δ 10.41 (s, 1H); 8.29 (d, J=5.8 Hz, 1H); 7.81 (s, 3H); 7.51 (s, 1H); 7.41 (d, J=5.7 Hz, 1H); 4.09 (d, J=13.1 Hz, 2H); 3.23-3.25 (m, 2H); 2.65 (t, J=11.2 Hz, 1H); 2.40 (s, 3H); 1.93 (d, J=13.2 Hz, 2H); 1.66 (qd, J=12.3, 4.1 Hz, 2H).

### Example 36

### Preparation of Compound 239—1-(3,5-dichlorophenyl)-3-(2-(2-methylpyridin-4-yl)-2-azaspiro[3.3]heptan-6-yl)urea, TFA

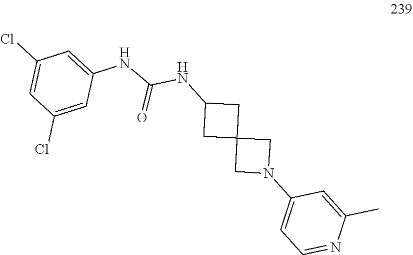 [SMILES: Cc1cc(N2CC3(CC(NC(=O)Nc4cc(Cl)cc(Cl)c4)C3)C2)ccn1]

### Step 1:

A solution of 1,3-dichloro-5-isocyanatobenzene (0.019 g, 0.1 mmol) in DCM (1.000 mL) was added to a vial containing tert-butyl 6-amino-2-azaspiro[3.3]heptane-2-carboxylate (0.025 g, 0.12 mmol). DIEA (0.050 ml, 0.286 mmol) was added, and the resulting mixture was allowed to stir at room temperature for 1.5 hours. The reaction mixture was concentrated in vacuo. MS ESI calc'd. for C₁₈H₂₃Cl₂N₃O₃ [M+H]⁺ 400, found 344.

### Step 2:

Tert-butyl 6-(3-(3,5-dichlorophenyl)ureido)-2-azaspiro[3.3]heptane-2-carboxylate (40 mg, 0.100 mmol) was dissolved in MeOH (1 mL). HCl (4M in Dioxane) (1 ml, 4.00 mmol) was added, and the resulting mixture was allowed to stir for 1 hour at room temperature. The reaction mixture was concentrated in vacuo. MS ESI calc'd. for C₁₃H₁₅Cl₂N₃₀ [M+H]⁺ 300, found 300.

### Step 3:

A solution of 4-bromo-2-methylpyridine (15.86 μl, 0.134 mmol) in THF (446 μl) was added to a vial containing 1-(3,5-dichlorophenyl)-3-(2-azaspiro[3.3]heptan-6-yl)urea, HCl (30 mg, 0.089 mmol), and 3rd Gen RuPhos Precatalyst (11.18 mg, 0.013 mmol). The vial was sealed and its contents were placed under an inert atmosphere by performing 3 vacuum/nitrogen cycles. Sodium tert-butoxide (446 μl, 0.891 mmol) was added through the septum and the resulting mixture was allowed to stir at 60° C. for 1 hour. The reaction mixture was quenched with methanol and concentrated in vacuo. The reaction mixture was filtered, and purified using mass triggered reverse phase HPLC (ACN/water with 0.1% TFA modifier). The purified fractions were lyophilized via reduced pressure to provide 1-(3,5-dichlorophenyl)-3-(2-(2-methylpyridin-4-yl)-2-azaspiro[3.3]heptan-6-yl)urea, TFA salt. MS ESI calc'd. for C₁₉H₂₀Cl₂N₄O [M+H]⁺ 391, found 391. ¹H NMR (600 MHz, dmso): δ 13.14 (s, 1H); 9.00 (s, 1H); 8.04 (s, 1H); 7.43 (s, 2H); 7.03 (s, 1H); 6.89 (d, J=7.3 Hz, 1H); 6.45 (d, J=4.6 Hz, 2H); 4.21 (d, J=15.3 Hz, 2H); 4.10 (d, J=15.3 Hz, 2H); 4.00 (br s, 1H); 2.53 (t, J=9.2 Hz, 2H); 2.38 (d, J=3.2 Hz, 3H); 2.17 (t, J=9.3 Hz, 2H).

The following compound was prepared according to the method described in Example 36

| No. | | [M + H] + Obs′d |
|---|---|---|
| 240 | 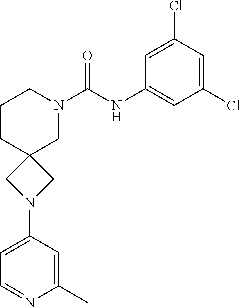 [SMILES: Cc1cc(N2CC3(CCCN(C(=O)Nc4cc(Cl)cc(Cl)c4)C3)C2)ccn1] | 405 |

### Example 37

### Preparation of Compound 241—3-(3,5-dichlorophenyl)-5-(2-(tetrahydro-2H-pyran-4-yl)-2,6-diazaspiro[3.5]nonan-6-yl)-1,2,4-oxadiazole

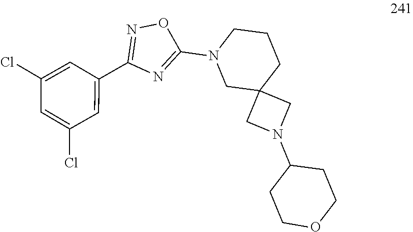 [SMILES: Clc1cc(Cl)cc(-c2noc(N3CCCC4(C3)CN(C3CCOCC3)C4)n2)c1]

### Step 1:

A solution of tert-butyl 2,6-diazaspiro[3.5]nonane-2-carboxylate, oxalic acid (114 mg, 0.360 mmol) in DMF (3000 μl) was added to a vial containing potassium carbonate (83 mg, 0.600 mmol), and 5-chloro-3-(3,5-dichlorophenyl)-1,2,4-oxadiazole (74.8 mg, 0.3 mmol). The resulting mixture was allowed to stir for 8 hours at 23° C. at which point a precipitate had formed. Water was added to the precipitate and the solids were collected by vacuum filtration and dried to provide tert-butyl 6-(3-(3,5-dichlorophenyl)-1,2,4-oxadiazol-5-yl)-2,6-diazaspiro[3.5]nonane-2-carboxylate. MS ESI calc'd. for C₂₀H₂₄Cl₂N₄O₃ [M+H]⁺ 439, found 383. ¹H NMR (600 MHz, dmso): δ 7.81-7.81 (m, 3H); 3.68 (br s, 2H); 3.52 (br s, 6H); 1.75 (s, 2H); 1.57 (s, 2H); 1.34 (s, 9H).

### Step 2:

4M HCl in dioxane (1.5 ml, 6.00 mmol) was added to a solution of tert-butyl 6-(3-(3,5-dichlorophenyl)-1,2,4-oxadiazol-5-yl)-2,6-diazaspiro[3.5]nonane-2-carboxylate (135 mg, 0.307 mmol) in MeOH (4 mL). The resulting mixture was allowed to stir for 3 hours at 23° C. The reaction mixture was concentrated in vacuo to provide 3-(3,5-dichlorophenyl)-5-(2,6-diazaspiro[3.5]nonan-6-yl)-1,2,4-oxadiazole, 2HCl salt. MS ESI calc'd. for C15H16Cl2N4O.2HCl [M+H]⁺ 339, found 339. ¹H NMR (600 MHz, dmso): δ 9.00 (br s, 1H); 8.85 (br s, 1H); 7.82 (s, 3H); 3.80 (s, 2H); 3.63-3.68 (br m, 4H); 3.49 (t, J=5.6 Hz, 2H); 1.82 (t, J=5.7 Hz, 2H); 1.58 (d, J=6.6 Hz, 2H).

### Step 3:

A vial was charged with molecular sieves, 3-(3,5-dichlorophenyl)-5-(2,6-diazaspiro[3.5]nonan-6-yl)-1,2,4-oxadiazole, 2HCl (41.2 mg, 0.1 mmol), tetrahydro-4 h-pyran-4-one (0.014 ml, 0.150 mmol), DCE (1 mL), and acetic acid (0.050 mL). The resulting mixture (as a white slurry) was allowed to stir for 3 hours at 50° C. before sodium triacetoxyborohydride (31.8 mg, 0.150 mmol) was added. Upon addition of the reductant, the reaction mixture all went into solution. The reaction mixture was allowed to stir overnight at 50° C. The reaction mixture was quenched with MeOH, and concentrated in vacuo. The crude product was dissolved in DMSO, filtered, and purified using mass triggered reverse phase HPLC (MeCN/water with 0.1% TFA modifier). The purified fractions were lyophilized via reduced pressure to provide 3-(3,5-dichlorophenyl)-5-(2-(tetrahydro-2 h-pyran-4-yl)-2,6-diazaspiro[3.5]nonan-6-yl)-1,2,4-oxadiazole, TFA. MS ESI calc'd. for C₂₀H₂₄Cl₂N₄O₂ 423, found 423. 1H NMR (600 MHz, DMSO-d6) δ 9.96-9.81 (m, 2H), 7.86-7.80 (m, 2H), 4.02-3.98 (m, 1H), 3.97-3.89 (m, 3H), 3.89-3.84 (m, 1H), 3.82 (s, 1H), 3.75 (s, 1H), 3.56-3.48 (m, 3H), 3.21 (dt, J=18.7, 9.4 Hz, 3H), 1.92-1.87 (m, 1H), 1.86-1.78 (m, 3H), 1.71-1.63 (m, 1H), 1.59-1.51 (m, 1H), 1.33-1.23 (m, 2H).

### Example 38

### Preparation of Compound 242—3-(3,5-dimethoxyphenyl)-5-(1-ethyl-9-(2-methylpyridin-4-yl)-1,4,9-triazaspiro[5.5]undecan-4-yl)-1,2,4-oxadiazole, TFA salt

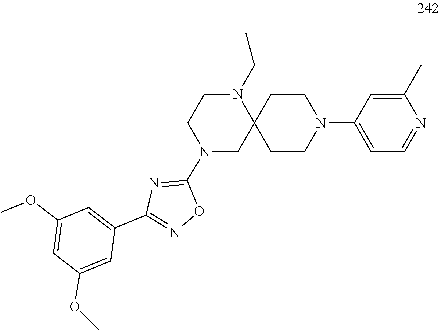

### Step 1:

A solution of 5-chloro-3-(3,5-dimethoxyphenyl)-1,2,4-oxadiazole in THF (2 mL) was added to a vial containing 1-benzyl 9-tert-butyl 1,4,9-triazaspiro[5.5]undecane-1,9-dicarboxylate and DIEA. The resulting mixture was allowed to stir overnight at 23° C. The reaction mixture was concentrated in vacuo to provide 1-benzyl 9-tert-butyl 4-(3-(3,5-dimethoxyphenyl)-1,2,4-oxadiazol-5-yl)-1,4,9-triazaspiro[5.5]undecane-1,9-dicarboxylate. MS ESI calc'd. for C₃₁H₃₉N₅O₇ [M+H]⁺ 594, found 594.

### Step 2:

TFA (1.5 ml, 19.47 mmol) was added to a solution of 1-benzyl 9-tert-butyl 4-(3-(3,5-dimethoxyphenyl)-1,2,4-oxadiazol-5-yl)-1,4,9-triazaspiro[5.5]undecane-1,9-dicarboxylate (250 mg, 0.421 mmol) in DCM (2 mL). The resulting mixture was allowed to stir for 2 hours at room temperature. The reaction mixture was concentrated in vacuo. The product was reconstituted in DCM and washed with saturated sodium bicarbonate (3×), and brine (lx) to provide benzyl 4-(3-(3,5-dimethoxyphenyl)-1,2,4-oxadiazol-5-yl)-1,4,9-triazaspiro[5.5]undecane-1-carboxylate. MS ESI calc'd. for C₃₁H₃₉N₅O₇ [M+H]⁺ 494, found 494.

### Step 3:

Benzyl 4-(3-(3,5-dimethoxyphenyl)-1,2,4-oxadiazol-5-yl)-1,4,9-triazaspiro[5.5]undecane-1-carboxylate (200 mg, 0.405 mmol), 4-bromo-2-methylpyridine (139 mg, 0.810 mmol), and 3rd Gen RuPhos Precatalyst (67.8 mg, 0.081 mmol) were added to a vial. The vial was sealed and its contents were placed under an inert atmosphere by performing 3 vacuum/nitrogen cycles. Sodium tert-butoxide (2M in THF) (405 μl, 0.810 mmol), and THF (2000 μl) were added through the septum and the resulting mixture was allowed to stir overnight at 80° C. The residue was purified using column chromatography on silica (0-100% 3:1 Ethyl Acetate:Ethanol/Hexane). The desired fractions were pooled and concentrated in vacuo to provide benzyl 4-(3-(3,5-dimethoxyphenyl)-1,2,4-oxadiazol-5-yl)-9-(2-methylpyridin-4-yl)-1,4,9-triazaspiro[5.5]undecane-1-carboxylate. MS ESI calc'd. for C₃₂H₃₆N₆O₅ [M+H]⁺ 585, found 585.

### Step 4:

Benzyl 4-(3-(3,5-dimethoxyphenyl)-1,2,4-oxadiazol-5-yl)-9-(2-methylpyridin-4-yl)-1,4,9-triazaspiro[5.5]undecane-1-carboxylate (95 mg, 0.162 mmol) was dissolved in Ethanol (2 mL). Pd—C (10%) (17.29 mg, 0.016 mmol) was added the flask was sealed and its contents were placed under an atmosphere of hydrogen by performing 3 vacuum/hydrogen balloon cycles. The contents of the flask were allowed to stir for 30 minutes at room temperature. The reaction was degassed with nitrogen, filtered, and concentrated. The residue was purified using column chromatography on silica (0-100% 3:1 Ethyl Acetate:Ethanol/Hexane with 10% DIEA). The desired fractions were pooled and concentrated in vacuo to provide 3-(3,5-dimethoxyphenyl)-5-(9-(2-methylpyridin-4-yl)-1,4,9-triazaspiro[5.5]undecan-4-yl)-1,2,4-oxadiazole. MS ESI calc'd. for C₂₄H₃₀N₆O₃ [M+H]⁺ 451, found 451.

Step 5: 3-(3,5-dimethoxyphenyl)-5-(9-(2-methylpyridin-4-yl)-1,4,9-triazaspiro[5.5]undecan-4-yl)-1,2,4-oxadiazole (20 mg, 0.044 mmol), acetaldehyde (9.78 mg, 0.222 mmol), 3 A molecular sieves, DCE (500 μL), and Acetic Acid (25.00 μL) were added to a vial. The contents of the vial were allowed to stir at 50° C. for 30 minutes, sodium triacetoxyborohydride (18.82 mg, 0.089 mmol) was added, and the resulting mixture was allowed to stir for an additional 3 hours at 50° C. The reaction mixture was filtered, and submitted directly for HPLC purification to the HTP group (purified using HPLC, eluting acetonitrile/water gradient with 0.1% TFA modifier, linear gradient), and lyophilized to provide 3-(3,5-dimethoxyphenyl)-5-(1-ethyl-9-(2-methylpyridin-4-yl)-1,4,9-triazaspiro[5.5]undecan-4-yl)-1,2,4-oxadiazole, TFA. MS ESI calc'd. for C₂₆H₃₄N₆O₃ [M+H]⁺ 479, found 479. 1H NMR (499 MHz, DMSO-d6) δ 13.53 (s, 1H), 10.18 (s, 1H), 8.21 (s, 1H), 7.13 (s, 1H), 7.11 (d, J=7.0 Hz, 1H), 7.04 (d, J=1.8 Hz, 2H), 6.71 (s, 1H), 4.37 (s, 2H), 4.13 (s, 4H), 3.80 (s, 6H), 3.61-3.37 (m, 4H), 3.06 (s, 1H), 2.47 (s, 3H), 2.31-1.88 (m, 4H), 1.27 (s, 3H).

### Example 39

### Preparation of Compound 243—(3-methoxy-5-(5-(1-methyl-9-(2-methylpyridin-4-yl)-1,4,9-triazaspiro[5.5]undecan-4-yl)-1,2,4-oxadiazol-3-yl)phenyl)methanamine

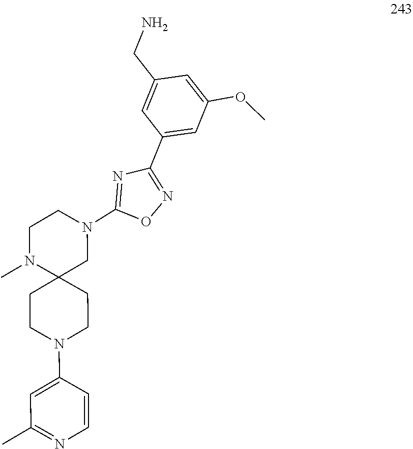

### Step 1:

BH₃THF (2.83 mL, 2.83 mmol)(1 M in THF) was added to a stirred mixture of 3-bromo-5-methoxybenzonitrile (200 mg, 0.943 mmol) in THF (5 mL), and the reaction was allowed to stir at 15° C. for 8 hours. The mixture was cooled to 0° C., MeOH (5 mL) was added, and stirred at room temperature for 10 minutes to quench the BH₃, then Boc₂O (0.263 mL, 1.132 mmol) and TEA (0.394 mL, 2.83 mmol) were added, and the reaction was allowed to stir 15° C. for 2 hours. The reaction mixture was concentrated in vacuo to provide a crude residue, which was purified using flash silica gel chromatography (ISCO; 4 g Agela Silica Flash Column, Eluent of 0-10% EA/PE gradient @ 30 mL/min) to provide tert-butyl 3-bromo-5-methoxybenzylcarbamate.

### Step 2:

Tetrakis(triphenylphosphine)palladium(0) (0.987 g, 0.854 mmol) was added to a stirred mixture of tert-butyl 3-bromo-5-methoxybenzylcarbamate (2.7 g, 8.54 mmol) and Zn(CN)₂ (3.01 g, 25.6 mmol) in DMF (30 mL). The resulting suspension was degassed and backfilled with Argon three times, and the resulting reaction was heated with stirring to 90° C., and allowed to stir at this temperature for 8 hours. The mixture was cooled to room temperature, water (150 mL) was added, and the reaction was extracted with ethyl acetate (2×100 mL). The combined organic extracts were washed with brine (300 mL), dried (Na₂SO₄), filtered and the solvent was removed in vacuo to provide a crude residue which was purified using flash silica gel chromatography (ISCO; 12 g Agela Silica Flash Column, Eluent of 0-18% EA/PE gradient @ 30 mL/min) to provide tert-butyl 3-cyano-5-methoxybenzylcarbamate.

### Step 3:

Hydroxylamine hydrochloride (0.344 g, 4.96 mmol) was added to a mixture of tert-butyl 3-cyano-5-methoxybenzylcarbamate (1 g, 3.81 mmol) and triethylamine (1.594 mL, 11.44 mmol) in MeOH (10 mL) at 50° C., and the reaction was allowed to stir at 50° C. for 8 hours. The mixture was cooled to room temperature then concentrated in vacuo to provide a residue. Water (50 mL) and EtOAc (50 mL) were added, the reaction was filtered, and the filter cake was washed with water (60 mL), followed by drying under vacuum to provide (Z)-tert-butyl 3-(N-hydroxycarbamimidoyl)-5-methoxybenzylcarbamate, which was used without further purification.

### Step 4:

Di(1H-imidazol-1-yl)methanethione (710 mg, 3.98 mmol) was added to a stirred mixture of (Z)-tert-butyl 3-(N′-hydroxycarbamimidoyl)-5-methoxybenzylcarbamate (980 mg, 3.32 mmol) in dioxane (10 mL), and the reaction was heated with stirring at 100° C. for 2 hours. The mixture was cooled to room temperature, then MeI (1.660 mL, 26.5 mmol) was added, and the reaction was allowed to stir at 25° C. for 8 hours. The reaction mixture was concentrated in vacuo to provide a crude residue, which was purified using flash silica gel chromatography (ISCO; 4 g Agela Silica Flash Column, Eluent of 0-10% EA/PE gradient @ 30 mL/min) to provide tert-butyl 3-methoxy-5-(5-(methylthio)-1,2,4-oxadiazol-3-yl)benzylcarbamate.

### Step 5:

mCPBA (552 mg, 2.56 mmol)(80%) was added to a stirred mixture of tert-butyl 3-methoxy-5-(5-(methylthio)-1,2,4-oxadiazol-3-yl)benzylcarbamate (300 mg, 0.854 mmol) in dichloromethane (10 mL), and the reaction was allowed to stir at 25° C. for 8 hours. Aqueous NaHCO₃ (saturated, 20 mL) was added, and the reaction was extracted with dichloromethane (2×10 mL). The combined organic extracts were washed with brine (saturated, 20 mL), dried (Na₂SO₄), filtered, and the solvent was removed in vacuo to provide tert-butyl 3-methoxy-5-(5-(methylsulfonyl)-1,2,4-oxadiazol-3-yl)benzylcarbamate, which was used without further purification.

### Step 6:

Triethylamine (0.161 mL, 1.152 mmol) was added to a stirred mixture of l-methyl-9-(2-methylpyridin-4-yl)-1,4,9-triazaspiro[5.5]undecane (50 mg, 0.192 mmol) and tert-butyl 3-methoxy-5-(5-(methylsulfonyl)-1,2,4-oxadiazol-3-yl)benzylcarbamate (73.6 mg, 0.192 mmol) in DMF (4 mL), and the resulting reaction was allowed to stir at room temperature for 30 minutes. The reaction mixture was purified using prep-HPLC (Column Waters XSELECT C18 150×30 mm×5 m, water (0.1% TFA)-MeCN) to provide tert-butyl (3-methoxy-5-(5-(1-methyl-9-(2-methylpyridin-4-yl)-1,4,9-triazaspiro[5.5]undecan-4-yl)-1,2,4-oxadiazol-3-yl)phenyl)carbamate.

### Step 7:

HCl (1 mL, 4.00 mmol)(4 M in dioxane) was added to a mixture of tert-butyl 3-methoxy-5-(5-(1-methyl-9-(2-methylpyridin-4-yl)-1,4,9-triazaspiro[5.5]undecan-4-yl)-1,2,4-oxadiazol-3-yl)benzylcarbamate (50 mg, 0.089 mmol) in MeOH (2 mL). The resulting suspension was allowed to stir at 40° C. for 1 hour, then the reaction mixture was removed in vacuo to provide compound 243. 1H NMR (400 MHz, MeOD) δ 8.08 (br d, J=6.1 Hz, 1H), 7.71 (s, 1H), 7.55 (s, 1H), 7.24 (s, 1H), 7.15-7.05 (m, 4H), 4.34-3.94 (m, 8H), 3.89 (s, 3H), 3.70-3.55 (m, 4H), 3.03 (s, 3H), 2.56 (s, 3H), 2.46-2.10 (m, 4H).

### Example 40

### Preparation of Compound 244—1-(3-methoxy-5-(5-(1-methyl-9-(2-methylpyridin-4-yl)-1,4,9-triazaspiro[5.5]undecan-4-yl)-1,2,4-oxadiazol-3-yl)phenyl)-N,N-dimethylmethanamine

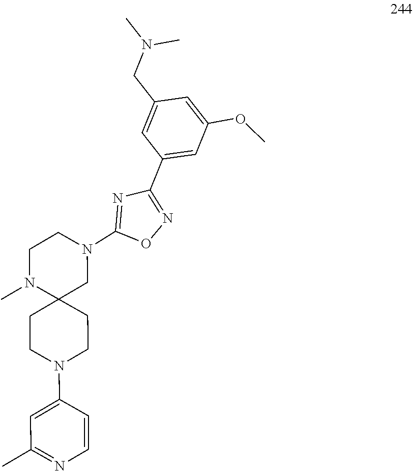

To a solution of (3-methoxy-5-(5-(1-methyl-9-(2-methylpyridin-4-yl)-1,4,9-triazaspiro[5.5]undecan-4-yl)-1,2,4-oxadiazol-3-yl)phenyl)methanamine (20 mg, 0.043 mmol) and paraformaldehyde (19.43 mg, 0.216 mmol) in MeOH (1.5 mL) was added acetic acid (0.020 mL, 0.345 mmol). The reaction mixture was allowed to stir at 25° C. for 20 minutes, then sodium triacetoxyhydroborate (73.1 mg, 0.345 mmol) was added, and the reaction was allowed to stir for another 1 hour. Additional triacetoxyhydroborate (45.7 mg, 0.216 mmol) was added, and the reaction mixture was allowed to stir for an additional 1 hour. The reaction mixture was purified using prep-HPLC(Column Waters XSELECT C18 150×30 mm×5 m, water (0.1% TFA)-MeCN) to provide compound 244. LCMS (ESI) calc'd for C₂₇H₃₇N₇O₂ [M+H]⁺: 492.3, found: 492.3. ¹H NMR (400 MHz, MeOD) δ 8.06 (d, J=7.5 Hz, 1H), 7.71 (s, 1H), 7.63 (s, 1H), 7.26 (s, 1H), 7.09-7.04 (m, 2H), 4.36 (s, 2H), 4.19-4.01 (m, 6H), 3.90 (s, 3H), 3.66-3.53 (m, 4H), 2.98 (s, 3H), 2.88 (s, 6H), 2.54 (s, 3H), 2.30-2.16 (m, 4H).

### Example 41

### Preparation of Compound 245—1-(3-methoxy-5-(5-(1-methyl-9-(2-methylpyridin-4-yl)-1,4,9-triazaspiro[5.5]undecan-4-yl)-1,2,4-oxadiazol-3-yl)phenyl)-N-methylmethanamine, HCl

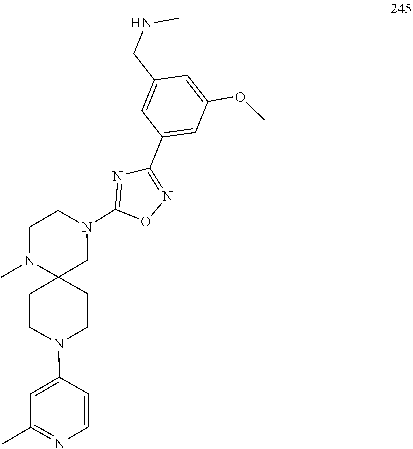

### Step 1:

NaH (29.6 mg, 0.740 mmol)(60% mineral oil dispersion) was added to a stirred mixture of tert-butyl 3-methoxy-5-(5-(methylthio)-1,2,4-oxadiazol-3-yl)benzylcarbamate (130 mg, 0.370 mmol) in DMF (2 mL), and the reaction was allowed to stir at −50° C. for 30 minutes. MeI (0.093 mL, 1.480 mmol) was added, and the reaction was allowed to stir at −50° C. for 6 hours. Water (20 mL) was then added, and the resulting solution was extracted with ethyl acetate (2×15 mL). The combined organic extracts were washed with brine (saturated, 30 mL), dried (Na₂SO₄), filtered, and concentrated in vacuo. The residue obtained was purified using preparative TLC on silica gel, eluting with petroleum ether/EtOAc=3:1(v/v) to provide tert-butyl 3-methoxy-5-(5-(methylthio)-1,2,4-oxadiazol-3-yl)benzyl(methyl)carbamate.

### Step 2:

mCPBA (124 mg, 0.575 mmol)(80%) was added to a stirred mixture of tert-butyl 3-methoxy-5-(5-(methylthio)-1,2,4-oxadiazol-3-yl)benzyl(methyl)carbamate (70 mg, 0.192 mmol) in dichloromethane (3 mL), and the reaction was allowed to stir at 25° C. for 8 hours. The reaction mixture was concentrated in vacuo to provide tert-butyl 3-methoxy-5-(5-(methylsulfonyl)-1,2,4-oxadiazol-3-yl)benzyl(methyl)carbamate, which was used without further purification.

### Step 3:

Triethylamine (0.161 mL, 1.152 mmol) was added to a stirred mixture of 1-methyl-9-(2-methylpyridin-4-yl)-1,4,9-triazaspiro[5.5]undecane (30 mg, 0.115 mmol) and tert-butyl 3-methoxy-5-(5-(methylsulfonyl)-1,2,4-oxadiazol-3-yl)benzyl(methyl)carbamate (68.7 mg, 0.173 mmol) in DMF (3 mL), and the resulting reaction was allowed to stir at room temperature for 30 minutes. The reaction mixture was then purified using prep-HPLC(Column Waters XSELECT C18 150×30 mm×5 μm, water (0.1% TFA)-MeCN) to provide tert-butyl (3-methoxy-5-(5-(1-methyl-9-(2-methylpyridin-4-yl)-1,4,9-triazaspiro[5.5]undecan-4-yl)-1,2,4-oxadiazol-3-yl)phenyl)(methyl)carbamate.

### Step 4:

HCl (1 mL, 4.00 mmol)(4 M in dioxane) was added to a mixture of tert-butyl 3-methoxy-5-(5-(1-methyl-9-(2-methylpyridin-4-yl)-1,4,9-triazaspiro[5.5]undecan-4-yl)-1,2,4-oxadiazol-3-yl)benzyl(methyl)carbamate (20 mg, 0.035 mmol) in MeOH (2 mL). The resulting suspension was allowed to stir at 40° C. for 1 hour. The reaction mixture was concentrated in vacuo to provide compound 245 which was used without further purification. LCMS (ESI) calc'd for C₂₆H₃₅N₇O₂ [M+H]⁺: 478.3, found: 478.3. ¹H NMR (400 MHz, MeOD) δ 8.07 (br d, J=7.5 Hz, 1H), 7.74 (s, 1H), 7.58 (s, 1H), 7.27 (s, 1H), 7.14-7.07 (m, 2H), 4.46-3.97 (m, 8H), 3.90 (s, 3H), 3.60-3.57 (m, 4H), 3.03 (s, 3H), 2.75 (s, 3H), 2.56 (s, 3H), 2.49-2.14 (m, 4H).

### Example 42

### Preparation of Compound 246—3-methoxy-5-(5-(1-methyl-9-(2-methylpyridin-4-yl)-1,4,9-triazaspiro[5.5]undecan-4-yl)-1,2,4-oxadiazol-3-yl)benzamide

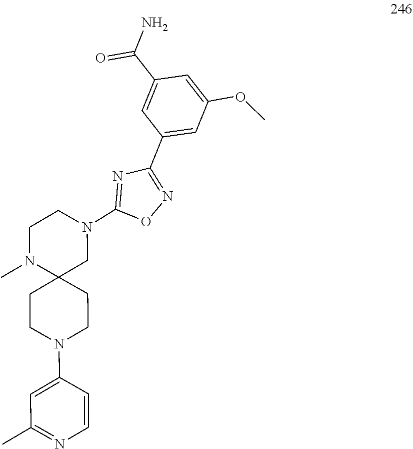

### Step 1:

A solution of 3-bromo-5-methoxybenzonitrile (3 g, 14.15 mmol), potassium acetate (4.17 g, 42.4 mmol) and PdCl₂(dppf) (0.518 g, 0.707 mmol) in EtOH (100 mL) was evacuated three times (backfilled with CO), and the resulting reaction was allowed to stir for 8 hours at 80° C. under 50 psi of CO. The reaction mixture was then cooled to room temperature and concentrated in vacuo to provide a crude residue, which was diluted with water (100 mL), and the resulting solution was extracted with ethyl acetate (2×50 mL). The combined organic extracts were washed with brine (saturated, 100 mL), dried (Na₂SO₄), filtered, and concentrated in vacuo. The resulting residue was washed with MeOH (10 mL), then dried under vacuum to provide a crude residue which was purified using flash silica gel chromatography (ISCO; 12 g Agela Silica Flash Column, Eluent of 0-50% EA/PE gradient @ 30 mL/min) to provide ethyl 3-cyano-5-methoxybenzoate.

### Step 2:

A mixture of ethyl 3-cyano-5-methoxybenzoate (600 mg, 2.92 mmol) in ammonium hydroxide solution (20.3 mL, 146 mmol)(28% in water) was allowed to stir at 45° C. for 8 hours. The reaction mixture was then filtered, and the filter cake was washed with water (30 mL) and dried under vacuum to provide 3-cyano-5-methoxybenzamide which was used without further purification.

### Step 3:

Hydroxylamine hydrochloride (149 mg, 2.140 mmol) was added to a mixture of 3-cyano-5-methoxybenzamide (290 mg, 1.646 mmol) and triethylamine (0.688 mL, 4.94 mmol) in MeOH (10 mL) at 50° C., and the resulting reaction was allowed to stir at 50° C. for 8 hours. The reaction mixture was cooled to room temperature, then concentrated in vacuo to provide a crude residue. Water (30 mL) and EtOAc (30 mL) were added to the crude residue, and the resulting solution was filtered. The filter cake was washed with water (30 mL), then dried under vacuum to provide (Z)-3-(N-hydroxycarbamimidoyl)-5-methoxybenzamide which was used without further purification.

### Step 4:

CDI (196 mg, 1.209 mmol) was added to a stirred mixture of (Z)-3-(N-hydroxycarbamimidoyl)-5-methoxybenzamide (230 mg, 1.099 mmol) in 1,4-dioxane (10 mL) and the resulting reaction was heated with stirring at 100° C. for 1 hour. The reaction mixture was then cooled to room temperature, filtered, and the filter cake was washed with 1,4-dioxane (10 mL). The filtrate was concentrated in vacuo to provide 3-(5-hydroxy-1,2,4-oxadiazol-3-yl)-5-methoxybenzamide as a solid, which was used without further purification.

### Step 5:

POCl₃ (0.040 mL, 0.425 mmol) was added dropwise to a stirred mixture of 3-methoxy-5-(5-oxo-4,5-dihydro-1,2,4-oxadiazol-3-yl)benzamide (50 mg, 0.213 mmol)) and pyridine (0.034 mL, 0.425 mmol) in toluene (3 mL)) at 0-5° C., and the resulting reaction was heated to 120° C., and allowed to stir at this temperature for 4 hours. The reaction mixture was cooled to room temperature, then the solvent was removed in vacuo to provide 3-(5-chloro-1,2,4-oxadiazol-3-yl)-5-methoxybenzonitrile, which was used without further purification.

### Step 6:

Triethylamine (0.032 mL, 0.230 mmol) was added to a stirred mixture of 1-methyl-9-(2-methylpyridin-4-yl)-1,4,9-triazaspiro[5.5]undecane (10 mg, 0.038 mmol) and 3-(5-chloro-1,2,4-oxadiazol-3-yl)-5-methoxybenzonitrile (18.1 mg, 0.077 mmol) in DMF (1 mL), and the reaction was allowed to stir at room temperature for 30 minutes. The reaction mixture was then purified using prep-HPLC(Column Boston Green ODS 150×30 5 g, water (0.1% TFA)-MeCN) to provide 3-methoxy-5-(5-(1-methyl-9-(2-methylpyridin-4-yl)-1,4,9-triazaspiro[5.5]undecan-4-yl)-1,2,4-oxadiazol-3-yl)benzonitrile.

### Step 7:

H₂O₂(0.017 mL, 0.196 mmol)(30% in water) was added to a stirred mixture of 3-methoxy-5-(5-(1-methyl-9-(2-methylpyridin-4-yl)-1,4,9-triazaspiro[5.5]undecan-4-yl)-1,2,4-oxadiazol-3-yl)benzonitrile (15 mg, 0.033 mmol) and K₂CO₃ (27.1 mg, 0.196 mmol) in DMSO (1 mL), and the reaction was allowed to stir at room temperature for 30 minutes. The reaction mixture was then purified using prep-HPLC(Column Waters XSELECT C18 150×30 mm×5 μm, water (0.1% TFA)-MeCN) to provide compound 246. LCMS (ESI) calc'd for C₂₅H₃₁N₇₀₃ [M+H]⁺: 478.2, found: 478.2. ¹H NMR (400 MHz, MeOD) δ 8.09-8.05 (m, 2H), 7.66 (s, 1H), 7.58 (s, 1H), 7.09-7.05 (m, 2H), 4.17-4.00 (m, 6H), 3.91 (s, 3H), 3.64 (br t, J=11.4 Hz, 2H), 3.57-3.47 (m, 2H), 3.00-2.91 (m, 3H), 2.54 (s, 3H), 2.30-2.11 (m, 4H).

### Example 43

### Preparation of Compound 247—3-(((3-(3,5-dimethoxyphenyl)-1,2,4-oxadiazol-5-yl)amino)methyl)-1-(2-methylpyridin-4-yl)azetidin-3-ol

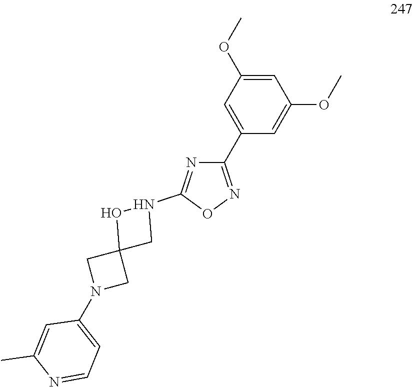

### Step 1:

A mixture of tert-butyl 3-(aminomethyl)-3-hydroxyazetidine-1-carboxylate (100 mg, 0.494 mmol), 5-chloro-3-(3,5-dimethoxyphenyl)-1,2,4-oxadiazole (119 mg, 0.494 mmol) and triethylamine (0.207 ml, 1.483 mmol) in DMF (4 mL) was allowed to stir at 30° C. for 2 hours. The reaction mixture was then concentrated in vacuo, and the residue obtained was purified using preparative TLC (SiO₂, Petroleum ether/EtOAc=1:1) to provide tert-butyl 3-(((3-(3,5-dimethoxyphenyl)-1,2,4-oxadiazol-5-yl)amino)methyl)-3-hydroxyazetidine-1-carboxylate.

### Step 2:

A mixture of tert-butyl 3-(((3-(3,5-dimethoxyphenyl)-1,2,4-oxadiazol-5-yl)amino)methyl)-3-hydroxyazetidine-1-carboxylate (50 mg, 0.123 mmol) in HCl/1,4-dioxane (4M) (4 ml, 4 mmol) was allowed to stir at 30° C. for 2 hours. The reaction mixture was then concentrated in vacuo to provide 3-(((3-(3,5-dimethoxyphenyl)-1,2,4-oxadiazol-5-yl)amino)methyl)azetidin-3-ol, which was used without purification.

### Step 3:

A 40-mL vial was charged with 3-(((3-(3,5-dimethoxyphenyl)-1,2,4-oxadiazol-5-yl)amino)methyl)azetidin-3-ol (30 mg, 0.098 mmol), 4-fluoro-2-methylpyridine (21.8 mg, 0.196 mmol), DIPEA (0.103 ml, 0.588 mmol) and DMA (1 mL). The reaction mixture was allowed to stir at 85° C. for 15 hours, then cooled to room temperature. The reaction was diluted with water (10 mL), then extracted with EtOAc (10 mL×3). The combined organic layers were concentrated and further purified using prep-HPLC (Column Boston Green ODS 150×30×5 μm, water (0.1% TFA)-MeCN) to provide compound 247. LCMS (ESI) calc'd for C₂₀H₂₄N₅O₄ [M+H]⁺: 398.2 found, 398.2 required. ¹H NMR (500 MHz, CDCl₃) δ 14.49 (br s, 1H), 7.86 (br s, 1H), 7.19 (br s, 1H), 7.07 (d, J=1.68 Hz, 2H), 6.55 (s, 1H), 6.24 (br d, J=5.80 Hz, 1H), 6.13 (s, 1H), 4.22 (br s, 2H), 4.14 (br d, J=10.22 Hz, 2H), 3.87 (br d, J=4.43 Hz, 2H), 3.81 (s, 5H), 3.83 (br s, 1H), 2.48 (s, 3H).

### Example 44

### PRDM9 Biochemical Assay

The PRDM9 biochemical assay was carried out using a radiometric format involving the transfer of ³[H] methyl group from S-adenosyl methionine (SAM) to a biotinylated histone H3 peptide (H3(1-25)K4). 10 mM compounds in DMSO were diluted 20 fold in assay buffer (10 mM Tris-HCl, pH 8.5, 10 mM DTT, and 0.01% Triton X-100) before adding 11l to a well in a 384 well polypropylene microplate plate and adding 8 μl of reaction mix (1.25 nM hPRDM9 (amino acids 195-385), 1.25 μM biotinylated H3 peptide in assay buffer). The ³[H]-SAM and cold SAM stock solutions were diluted to 50 μM and 550 μM respectively in assay buffer before aliquoting 1 μl of 50 μM ³[H]-SAM, 550 μM SAM into each well to initiate the reaction. The assay was performed at 25° C. at the final concentrations of 1 nM hPRDM9, 1 μM peptide, 5 μM ³[H]-SAM, and 55 μM un-labeled SAM in assay buffer. The reaction was quenched after 30 minutes by adding 25 μl 7.5 M Guanidine-HCl. Quenched reactions were transferred to streptavidin coated Flashplates, diluted two fold in assay buffer, incubated for 1 hour, and read on a Perkin Elmer Topcount instrument. Compound activity was expressed relative to activity of 0.5% (v/v) DMSO treated samples and fit to a four parameter logistic equation to determine IC₅₀ values.

Illustrative compounds of the present invention were tested using this assay protocol and results are presented in the Table below.

| Compound No. | PRDM9 IC₅₀ (nM) |
|---|---|
| 1 | 5160 |
| 2 | 2400 |
| 3 | 3390 |
| 4 | 360 |
| 5 | 16200 |
| 6 | 11300 |
| 7 | 4850 |
| 8 | 804 |
| 9 | 42600 |
| 10 | 50000 |
| 11 | 19700 |
| 12 | 41500 |
| 13 | 5160 |
| 14 | 297 |
| 15 | 31600 |
| 16 | 11500 |
| 17 | 27900 |
| 18 | 10600 |
| 19 | 25700 |
| 20 | 7790 |
| 21 | 2750 |
| 22 | 18600 |
| 23 | 17800 |
| 24 | 5340 |
| 25 | 13900 |
| 26 | 370 |
| 27 | 36400 |
| 28 | 22.5 |
| 29 | 13300 |
| 30 | 28200 |
| 31 | 11300 |
| 32 | 2210 |
| 33 | 1520 |
| 34 | 2200 |
| 35 | 5580 |
| 36 | 27800 |
| 37 | 15500 |
| 38 | 160 |
| 39 | 12300 |
| 40 | 80.3 |
| 41 | 3920 |
| 42 | 35800 |
| 43 | 13300 |
| 44 | 500 |
| 45 | 340 |
| 46 | 350 |
| 47 | 11300 |
| 48 | 1560 |
| 49 | 40200 |
| 50 | 16400 |
| 51 | 7290 |
| 52 | 29400 |
| 53 | 3390 |
| 54 | 26100 |
| 55 | 10700 |
| 56 | 28000 |
| 57 | 17000 |
| 58 | 24000 |
| 59 | 21100 |
| 60 | 170 |
| 61 | 8000 |
| 62 | 22400 |
| 63 | 44100 |
| 64 | 3580 |
| 65 | 6200 |
| 66 | 470 |
| 67 | 4440 |
| 68 | 2800 |
| 69 | 7040 |
| 70 | 6900 |
| 71 | 27700 |
| 72 | 441 |
| 73 | 36900 |
| 74 | 1130 |
| 75 | 18400 |
| 76 | 4330 |
| 77 | 2630 |
| 78 | 17800 |
| 79 | 19100 |
| 80 | 2830 |
| 81 | 13300 |
| 82 | 37300 |
| 83 | 50000 |
| 85 | 80 |
| 86 | 280 |
| 87 | 470 |
| 88 | 60 |
| 89 | 80 |
| 90 | 118 |
| 91 | 150 |
| 92 | 69 |
| 93 | 60 |
| 94 | 109 |
| 95 | 44 |
| 96 | 103 |
| 97 | 1370 |
| 98 | 95 |
| 99 | 70 |
| 100 | 140 |
| 101 | 450 |
| 102 | 4400 |
| 103 | 2630 |
| 104 | NA |
| 105 | NA |
| 106 | NA |
| 107 | NA |
| 108 | NA |
| 109 | NA |
| 110 | NA |
| 111 | NA |
| 112 | NA |
| 113 | NA |
| 114 | NA |
| 115 | NA |
| 116 | 72 |
| 117 | 2000 |
| 118 | 16630 |
| 119 | 300 |
| 120 | 12240 |
| 121 | 13700 |
| 122 | 30600 |
| 123 | 6670 |
| 124 | 17600 |
| 125 | 25100 |
| 126 | 11100 |
| 127 | 26900 |
| 128 | 23700 |
| 129 | 19100 |
| 130 | 24300 |
| 131 | 23100 |
| 132 | 5460 |
| 133 | 17000 |
| 134 | 34600 |
| 135 | 21100 |
| 136 | 16500 |
| 137 | 21500 |
| 138 | 4800 |
| 139 | 393 |
| 140 | 4800 |
| 142 | 203 |
| 143 | 50000 |
| 144 | 42300 |
| 145 | 4700 |
| 146 | 50000 |
| 147 | 263 |
| 148 | 50000 |
| 150 | 66 |
| 151 | 203 |
| 152 | 263 |
| 153 | 44800 |
| 154 | 3450 |
| 155 | 3460 |
| 156 | 16000 |
| 157 | 31000 |
| 158 | 3810 |
| 159 | 296 |
| 160 | 350 |
| 161 | 49 |
| 162 | 470 |
| 163 | 3075 |
| 164 | 315 |
| 166 | 800 |
| 167 | 23000 |
| 168 | 9000 |
| 169 | 2000 |
| 170 | 400 |
| 171 | 4000 |
| 172 | 5290 |
| 173 | 5500 |
| 174 | 1580 |
| 175 | 3810 |
| 176 | 13600 |
| 177 | 8640 |
| 178 | 1150 |
| 180 | 80 |
| 181 | 2140 |
| 182 | 163 |
| 184 | 814 |
| 186 | 217 |
| 188 | 45000 |
| 189 | 72 |
| 193 | 2000 |
| 196 | 19000 |
| 197 | 2000 |
| 200 | 19000 |
| 202 | 2000 |
| 203 | 1000 |
| 205 | 160 |
| 206 | 1600 |
| 207 | 400 |
| 208 | 5000 |
| 209 | 2000 |
| 210 | 35000 |
| 211 | 24000 |
| 212 | 24000 |
| 213 | 1000 |
| 214 | 464 |
| 215 | 11000 |
| 216 | 246 |
| 217 | 29200 |
| 218 | 35500 |
| 219 | 4300 |
| 220 | 9200 |
| 221 | 5380 |
| 222 | 13800 |
| 223 | 12100 |
| 224 | 12100 |
| 225 | 50000 |
| 227 | 35500 |
| 228 | 34300 |
| 230 | 11500 |
| 231 | 37300 |
| 232 | 2000 |
| 233 | 2750 |
| 234 | 8030 |
| 235 | 29500 |
| 236 | 400 |
| 237 | 7300 |
| 238 | 50000 |
| 239 | 50000 |
| 240 | 50000 |
| 242 | 155 |
| 243 | 103 |
| 244 | 131 |
| 245 | 83 |
| 246 | 83 |
| 247 | 650 |

### Treatment or Prevention of PRDM9-Mediated Disorders

The Substituted Heterocyclic Compounds are useful in the inhibition of PRDM9 activity, and the treatment or prevention of disorders related to the activity of PDRM9.

In one embodiment, the Substituted Heterocyclic Compounds are useful for treating cancer.

In another embodiment, the Substituted Heterocyclic Compounds are useful for male contraception.

Accordingly, in one embodiment, the invention provides methods for treating or preventing a disorder related to the activity of PDRM9 in a subject, the methods comprising administering to the subject an effective amount of at least one Substituted Heterocyclic Compound or a pharmaceutically acceptable salt or prodrug thereof. In one embodiment, the amount administered is effective to inhibit PRDM9 activity in said subject. In another embodiment, the amount administered is effective to treat cancer in a subject. In another embodiment, the amount administered is effective to cause contraception in a male subject.

The compositions and combinations of the present invention can be useful for treating a subject suffering from any disorder related to the activity of PRDM9.

### Methods For Treating or Preventing Cancer

The Substituted Heterocycle Compounds are useful for treating or preventing cancer in a patient.

Accordingly, in one embodiment, the present invention provides a method for treating cancer in a patient, comprising administering to the patient an effective amount of one or more Substituted Heterocycle Compounds.

Non-limiting examples of cancers treatable or preventable using the present methods include the following cancers and metastases thereof: bladder cancer, breast cancer, colorectal cancer, kidney cancer, liver cancer, non-small cell lung cancer, small cell lung cancer, non-small cell lung cancer, head and neck cancer, esophageal cancer, gall bladder cancer, ovarian cancer, pancreatic cancer, stomach cancer, cervical cancer, thyroid cancer, prostate cancer, skin cancer; hematopoietic tumors of lymphoid lineage, including leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including brain tumors (such as an astrocytoma, a neuroblastoma, a glioma or a schwannoma); and other tumors, including melanoma, seminoma, teratocarcinoma, osteosarcoma, xenoderoma pigmentosum, keratoctanthoma, thyroid follicular cancer and Kaposi's sarcoma. The Substituted Heterocycle Compounds are useful for treating primary tumors, metastatic tumors and tumors of unknown origin.

In one embodiment, the cancer treated is lung cancer.

In another embodiment, the cancer treated is breast cancer.

In another embodiment, the cancer treated is colorectal cancer.

In still another embodiment, the cancer treated is prostate cancer.

In another embodiment, the cancer treated is a leukemia.

In yet another embodiment, the cancer treated is a lymphoma.

In a further embodiment, the cancer treated is a metastatic tumor.

In one embodiment, the Substituted Heterocycle Compounds can be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

In another embodiment, the Substituted Heterocycle Compounds can be useful in inhibiting tumor angiogenesis and metastasis.

### Combination Therapy

In another embodiment, the present methods for treating or preventing cancer can further comprise the administration of one or more additional therapeutic agents which are not Substituted Heterocyclic Compounds.

Accordingly, in one embodiment, the present invention provides methods for treating cancer in a subject, the method comprising administering to the subject: (i) at least one Substituted Heterocyclic Compound (which may include two or more different Substituted Heterocyclic Compounds), or a pharmaceutically acceptable salt or prodrug thereof, and (ii) at least one additional therapeutic agent that is other than a Substituted Heterocyclic Compound, wherein the amounts administered are together effective to treat cancer.

When administering a combination therapy of the invention to a subject, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Substituted Heterocyclic Compound and an additional therapeutic agent may be present in fixed amounts (dosage amounts) in a single dosage unit (e.g., a capsule, a tablet and the like).

In one embodiment, at least one Substituted Heterocyclic Compound is administered during a time when the additional therapeutic agent(s) exert their prophylactic or therapeutic effect, or vice versa.

In another embodiment, at least one Substituted Heterocyclic Compound and the additional therapeutic agent(s) are administered in doses commonly employed when such agents are used as monotherapy for treating a viral infection.

In another embodiment, at least one Substituted Heterocyclic Compound and the additional therapeutic agent(s) are administered in doses lower than the doses commonly employed when such agents are used as monotherapy for treating a viral infection.

In still another embodiment, at least one Substituted Heterocyclic Compound and the additional therapeutic agent(s) act synergistically and are administered in doses lower than the doses commonly employed when such agents are used as monotherapy for treating a viral infection.

In one embodiment, at least one Substituted Heterocyclic Compound and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Cancers that can be treated using the combination therapy methods of the present invention include, but are not limited to, those listed above.

The at least one Substituted Heterocyclic Compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy. In one embodiment, the administration of at least one Substituted Heterocyclic Compound and the additional therapeutic agent(s) may inhibit the resistance of cancer to these agents.

Non-limiting examples of additional anticancer agents suitable for use in the present methods for treating cancer include cytostatic agents, cytotoxic agents (such as for example, but not limited to, DNA interactive agents (such as cisplatin or doxorubicin)); taxanes (e.g. taxotere, taxol); topoisomerase II inhibitors (such as etoposide or teniposide); topoisomerase I inhibitors (such as irinotecan (or CPT-11), camptostar, or topotecan); tubulin interacting agents (such as paclitaxel, docetaxel or the epothilones); hormonal agents (such as tamoxifen); thymidilate synthase inhibitors (such as 5-fluorouracil); anti-metabolites (such as methoxtrexate); alkylating agents (such as temozolomide (TEMODAR™ from Schering-Plough Corporation, Kenilworth, N.J.), cyclophosphamide); Farnesyl protein transferase inhibitors (such as, SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoethyl]-1-piperidinecarboxamide, tipifarnib (Zarnestra® or R115777 from Janssen Pharmaceuticals), L778,123 (a farnesyl protein transferase inhibitor from Merck & Co., Inc.), BMS 214662 (a farnesyl protein transferase inhibitor from Bristol-Myers Squibb Pharmaceuticals, Princeton, N.J.); signal transduction inhibitors (such as, Iressa (from Astra Zeneca Pharmaceuticals, England), Tarceva (EGFR kinase inhibitors), antibodies to EGFR (e.g., C225), GLEEVEC™ (C-abl kinase inhibitor from Novartis Pharmaceuticals, East Hanover, N.J.); interferons such as, for example, intron (Merck & Co., Inc.), Peg-Intron (Merck & Co., Inc.); hormonal therapy combinations; aromatase combinations; ara-C, adriamycin, cytoxan, and gemcitabine.

Other useful additional anticancer agents include but are not limited to Uracil mustard, Chlormethine, Ifosfamide, Melphalan, Chlorambucil, Pipobroman, Triethylenemelamine, ara-C, adriamycin, cytoxan, Clofarabine (Clolar® from Genzyme Oncology, Cambridge, Mass.), cladribine (Leustat® from Janssen-Cilag Ltd.), aphidicolon, rituxan (from Genentech/Biogen Idec), sunitinib (Sutent® from Pfizer), dasatinib (or BMS-354825 from Bristol-Myers Squibb), tezacitabine (from Aventis Pharma), Smll, fludarabine (from Trigan Oncology Associates), pentostatin (from BC Cancer Agency), triapine (from Vion Pharmaceuticals), didox (from Bioseeker Group), trimidox (from ALS Therapy Development Foundation), amidox, 3-AP (3-aminopyridine-2-carboxaldehyde thiosemicarbazone), MDL-101,731 ((E)-2′-deoxy-2′-(fluoromethylene)cytidine), and gemcitabine.

Other useful additional anticancer agents include but are not limited to Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaceuticals, France), LYNPARZA® (Astra Zeneca), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide, 17a-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Oxaliplatin, Aroplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux, Liposomal, Thiotepa, Altretamine, Melphalan, Trastuzumab, Lerozole, Fulvestrant, Exemestane, Fulvestrant, Ifosfomide, Rituximab, C225 and Campath.

Additional useful additional anticancer agents include but are not limited to immune-oncology agents, including, but are not limited to pembrolizumab, nivolumab, ipilimumab, durvalumab, atezolizumab, avelumab, epacadostat MK-4166, MK-7684 and MK-4280.

If formulated as a fixed dose, such combination products employ the compounds of this invention within the dosage range described herein and the additional anticancer agent(s) or treatment within its dosage range. For example, the CDC2 inhibitor olomucine has been found to act synergistically with known cytotoxic agents in inducing apoptosis (J Cell Sci., (1995) 108, 2897. Heterocyclic Compounds may also be administered sequentially with known anticancer or cytotoxic agents when a combination formulation is inappropriate. The invention is not limited in the sequence of administration; Heterocyclic Compounds may be administered either prior to or after administration of the known anticancer or cytotoxic agent. For example, the cytotoxic activity of the cyclin-dependent kinase inhibitor flavopiridol is affected by the sequence of administration with anticancer agents. Cancer Research, (1997) 57, 3375. Such techniques are within the skills of persons skilled in the art as well as attending physicians.

Accordingly, in an aspect, this invention includes methods for treating cancer in a patient, comprising administering to the patient an amount of at least one Heterocyclic Compound, or a pharmaceutically acceptable salt, solvate, ester, prodrug or stereoisomer thereof, and one or more other anticancer treatment modalities, wherein the amounts of the Heterocyclic Compound(s)/other treatment modality result in the desired therapeutic effect. In one embodiment, the at least one Heterocyclic Compound and the one or more other treatment modalities act synergistically. In one embodiment, the at least one Heterocyclic Compound and the one or more other treatment modalities act additively.

In one embodiment, the other treatment modality is surgery.

In another embodiment, the other treatment modality is radiation therapy.

In another embodiment, the other treatment modality is biological therapy, such as hormonal therapy or anticancer vaccine therapy.

In one embodiment, the present invention provides pharmaceutical compositions comprising (i) a compound of formula (I) or a pharmaceutically acceptable salt or prodrug thereof; (ii) a pharmaceutically acceptable carrier; and (iii) one or more additional anti-cancer agents selected from those listed above, or a pharmaceutically acceptable salt or prodrug thereof, wherein the amounts present of components (i), and (iii) are together effective for the treatment of cancer in the subject in need thereof.

It is understood that the scope of combinations of the compounds of this invention with anti-cancer agents is not limited to anti-cancer agents listed above herein, but includes in principle any combination with any pharmaceutical composition useful for the treatment of cancer. The anti-cancer agents will typically be employed in these combinations in their conventional dosage ranges and regimens as reported in the art, including, for example, the dosages described in the Physicians' Desk Reference, Thomson PDR, 69th edition (2016), 70th edition (2018), 71st edition (2017), and the like. The dosage ranges for a compound of the invention in these combinations are the same as those set forth above.

The doses and dosage regimen of the other agents used in the combination therapies of the present invention for the treatment of cancer can be determined by the attending clinician, taking into consideration the approved doses and dosage regimen in the package insert; the age, sex and general health of the subject; and the type and severity of the viral infection or related disease or disorder. When administered in combination, the Substituted Heterocyclic Compound(s), and the other agent(s) can be administered simultaneously (i.e., in the same composition or in separate compositions one right after the other) or sequentially. This is particularly useful when the components of the combination are given on different dosing schedules, e.g., one component is administered once daily and another component is administered every six hours, or when the pharmaceutical compositions are different, e.g., one is a tablet and one is a capsule. A kit comprising the separate dosage forms is therefore advantageous.

### COMPOSITIONS AND ADMINISTRATION

When administered to a subject, the Substituted Heterocyclic Compounds can be administered as a component of a composition that comprises a pharmaceutically acceptable carrier or vehicle. The present invention provides pharmaceutical compositions comprising an effective amount of at least one Substituted Heterocyclic Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms), and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., anti-cancer activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Substituted Heterocyclic Compounds are administered orally.

In another embodiment, the one or more Substituted Heterocyclic Compounds are administered intravenously.

In one embodiment, a pharmaceutical preparation comprising at least one

Substituted Heterocyclic Compound is in unit dosage form. In such form, the preparation is subdivided into unit doses containing effective amounts of the active components. Compositions can be prepared according to conventional mixing, granulating or coating methods, respectively, and the present compositions can contain, in one embodiment, from about 0.1% to about 99% of the Substituted Heterocyclic Compound(s) by weight or volume. In various embodiments, the present compositions can contain, in one embodiment, from about 1% to about 70% or from about 5% to about 60% of the Substituted Heterocyclic Compound(s) by weight or volume.

The Substituted Heterocyclic Compounds can be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the subject to be treated. The specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The unit dosages of the Substituted Heterocyclic Compounds may be administered at varying frequencies. In one embodiment, a unit dosage of a Substituted Heterocyclic Compound can be administered once daily. In another embodiment, a unit dosage of a Substituted Heterocyclic Compound can be administered twice weekly. In another embodiment, a unit dosage of a Substituted Heterocyclic Compound can be administered once weekly. In still another embodiment, a unit dosage of a Substituted Heterocyclic Compound can be administered once biweekly. In another embodiment, a unit dosage of a Substituted Heterocyclic Compound can be administered once monthly. In yet another embodiment, a unit dosage of a Substituted Heterocyclic Compound can be administered once bimonthly. In another embodiment, a unit dosage of a Substituted Heterocyclic Compound can be administered once every 3 months. In a further embodiment, a unit dosage of a Substituted Heterocyclic Compound can be administered once every 6 months. In another embodiment, a unit dosage of a Substituted Heterocyclic Compound can be administered once yearly.

The amount and frequency of administration of the Substituted Heterocyclic Compounds will be regulated according to the judgment of the attending clinician considering such factors as age, condition and size of the subject as well as severity of the symptoms being treated. The compositions of the invention can further comprise one or more additional therapeutic agents, selected from those listed above herein.

### Kits

In one aspect, the present invention provides a kit comprising a therapeutically effective amount of at least one Substituted Heterocyclic Compound, or a pharmaceutically acceptable salt or prodrug of said compound and a pharmaceutically acceptable carrier, vehicle or diluent.

In another aspect the present invention provides a kit comprising an amount of at least one Substituted Heterocyclic Compound, or a pharmaceutically acceptable salt or prodrug of said compound and an amount of at least one additional therapeutic agent listed above, wherein the amounts of the two or more active ingredients result in a desired therapeutic effect. In one embodiment, the one or more Substituted Heterocyclic Compounds and the one or more additional therapeutic agents are provided in the same container. In one embodiment, the one or more Substituted Heterocyclic Compounds and the one or more additional therapeutic agents are provided in separate containers.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

What is claimed is:

1. A compound having the formula:
R¹—R²—R³—R⁴ (I)
or a pharmaceutically acceptable salt thereof, wherein:
R¹ is selected from C₁-C₆ alkyl, C₁-C₆ hydroxyalkyl, C₆-C₁₀ aryl and C₃-C₆ cycloalkyl, wherein said C₁-C₆ alkyl group and C₆-C₁₀ aryl group is unsubstituted or substituted with up to three R^{A} groups;
R² is selected from 5 or 6-membered monocyclic heteroaryl, 5 or 6-membered cycloalkenyl, heterocycloalkyl, and —CH₂SO₂—, wherein said 5 or 6-membered monocyclic heteroaryl group, said 5 or 6-membered cycloalkenyl group, and said heterocycloalkyl group is unsubstituted or substituted with up to three R^{B} groups;
R³ is selected from —NR⁵—(C₁-C₆ alkylene)-NR⁵—, —(NR⁵)ᵣ—(C₁-C₃ alkylene)ₛ-(C₃-C₇ cycloalkyl)-(5 to 16-membered heterocycloalkyl)-(C₁-C₃ alkylene)ₛ-(NR⁵)ᵣ—, and —(NR⁵)ᵣ—(C₁-C₃ alkylene)ₛ-(5 to 16-membered heterocycloalkyl)-(C₁-C₃ alkylene)ₛ-(NR⁵)ᵣ, wherein said 5 to 16-membered heterocycloalkyl group must have at least one ring nitrogen atom and wherein said 5 to 16-membered heterocycloalkyl group, said C₁-C₆ alkylene group, and said C₃-C₇ cycloalkyl group is unsubstituted or substituted with up to three R^{C} groups;
R⁴ is selected from C₆-C₁₀ aryl, 5 or 6-membered monocyclic heteroaryl, 5 or 6-membered monocyclic heterocycloalkyl, and 9 or 10-membered bicyclic heteroaryl, wherein C₆-C₁₀ aryl group, said 5 or 6-membered monocyclic heteroaryl group, said 5 or 6-membered monocyclic heterocycloalkyl group, and said 9 or 10-membered bicyclic heteroaryl group is unsubstituted or substituted with up to three R^{D} groups;
each occurrence of R⁵ is independently selected from H, C₁-C₆ alkyl, and C₃-C₆ cycloalkyl;
each occurrence of R^{A} is independently selected from C₁-C₆ alkyl, C₁-C₆ alkylamino, —O—(C₁-C₆ alkyl), —O—(C₃-C₆ cycloalkyl), —O—(C₃-C₆ monocyclic cycloalkyl), halo, —OH, C₁-C₆ haloalkyl, C₁-C₆ hydroxyalkyl, —CN, —O—(C₁-C₆ haloalkyl), —C(O)O—(C₁-C₆ alkyl), —C(O)N(R⁵)₂, —(C₁-C₆ alkyl)-N(R⁵)₂, and —N(R⁵)₂;
each occurrence of R^{B} is independently selected from C₁-C₆ alkyl, halo, phenyl, and 5 or 6-membered monocyclic heteroaryl, wherein said phenyl group is unsubsituted or subtituted with up to three groups, each independently selected from C₁-C₆ alkyl, —O—(C₁-C₆ alkyl), halo, and —N(R⁵)₂;
each occurrence of R^{C} is independently selected from C₁-C₆ alkyl, —O—(C₁-C₆ alkyl), —(C₁-C₃ alkylene)-O—(C₁-C₆ alkyl), C₁-C₆ haloalkyl, C₁-C₆ hydroxyalkyl, —SO₂—(C₁-C₆ alkyl), phenyl, halo, —CN, —OH and —C(O)N(R⁵)₂;
each occurrence of R^{D} is independently selected from C₁-C₆ alkyl, —O—(C₁-C₆ alkyl), —O—(C₃-C₆ cycloalkyl), —O—(C₃-C₆ monocyclic cycloalkyl), halo, —OH, C₁-C₆ haloalkyl, C₁-C₆ hydroxyalkyl, —CN, —O—(C₁-C₆ haloalkyl), —C(O)O—(C₁-C₆ alkyl), —C(O)N(R⁵)₂ and —N(R⁵)₂;
each occurrence of r is independently 0 or 1; and
each occurrence of s is independently 0 or 1.

2. The compound of claim 1, wherein R¹ is phenyl, which is substituted with 1 or 2 groups, each independently selected from —(C₁-C₆ alkyl)-N(R⁵)₂, halo, —O—(C₁-C₆ alkyl), and —C(O)O—(C₁-C₆ alkyl), or a pharmaceutically acceptable salt thereof.

3. The compound of claim 1, wherein R² is selected from oxadiazolyl, isoxazolyl, pyrazolyl, triazolyl, and dihydropyrrolyl, or a pharmaceutically acceptable salt thereof, any of which can be optionally substituted with up to three R^{B} groups.

4. The compound of claim 1, wherein R³ is 8 to 16-membered bicyclic or tricyclic heterocycloalkyl, or a pharmaceutically acceptable salt thereof, either of which can be optionally substituted with up to three R^{C} groups.

5. The compound of claim 4, wherein R³ comprises a spirocyclic bicyclic heterocycloalkyl group, or a pharmaceutically acceptable salt thereof, which can be optionally substituted with up to three R^{C} groups.

6. The compound of claim 1, having the formula (Ia): 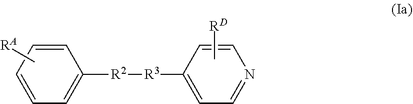
or a pharmaceutically acceptable salt thereof,
wherein:
R² is selected from oxadiazolyl, isoxazolyl, pyrazolyl, and dihydropyrrolyl, which is unsubsituted or subtituted with R^{B};
R³ is —NR⁵—(C₁-C₆ alkylene)-NR⁵—, —(NR⁵)ᵣ—(C₁-C₃ alkylene)ₛ-(5 or 6-membered monocyclic heterocycloalkyl)-(C₁-C₃ alkylene)ₛ-(NR⁵)ᵣ— or —(NR⁵)ᵣ—(C₁-C₃ alkylene)ₛ-(8 to 14-membered multicyclic heterocycloalkyl)-(C₁-C₃ alkylene)ₛ-(NR⁵)ᵣ—, wherein said 5 or 6-membered monocyclic heterocycloalkyl group and said 8 to 14-membered multicyclic heterocycloalkyl group must have at least one ring nitrogen atom and wherein said 5 or 6-membered monocyclic heterocycloalkyl group and said 8 to 14-membered multicyclic heterocycloalkyl group is unsubsituted or subtituted with R^{C};
each occurrence of R⁵ is independently selected from H, methyl and cyclopropyl;
R^{A} represents up to 2 ring substituents, each independently selected from —(C₁-C₆ alkyl)-N(R⁵)₂, Cl, F, methoxy and ethoxy;
R^{B} is independently selected from C₁-C₆ alkyl, halo, phenyl, and 5 or 6-membered monocyclic heteroaryl, wherein said phenyl group is unsubsituted or subtituted with up to three groups, each independently selected from C₁-C₆ alkyl, —O—C₁-C₆ alkyl, halo, and —N(R⁵)₂;
R^{C} represents up to 2 ring substituents, each independently selected from C₁-C₆ alkyl, methoxy, —(C₁-C₃ alkylene)-O—C₁-C₆ alkyl, C₁-C₆ haloalkyl, C₁-C₆ hydroxyalkyl, —SO₂—(C₁-C₆ alkyl), phenyl, halo, —CN, —OH and —C(O)N(R⁵)₂;
R^{D} represents up to 2 ring substituents, each independently selected from C₁-C₆ alkyl;
each occurrence of r is independently 0 or 1; and
each occurrence of s is independently 0 or 1.

7. The compound of claim 1, wherein R³ is: 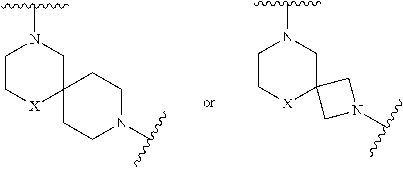
and X is selected from —O—, —NH, —N(CH₃)— and —CH₂—, or a pharmaceutically acceptable salt thereof.

8. The compound of claim 1, wherein R⁴ is: 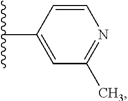 [SMILES: *c1ccnc(C)c1]
or a pharmaceutically acceptable salt thereof.

9. A compound being any of the compounds numbered 1-247 in the above specification, or a pharmaceutically acceptable salt thereof.

10. A pharmaceutical composition comprising an effective amount of a compound of claim 1, or a pharmaceutically acceptable salt thereof, and a pharmaceutically acceptable carrier.

11. A method for the inhibition of PRDM9 activity in a subject in need thereof which comprises administering to the subject an effective amount of the compound of claim 1, or a pharmaceutically acceptable salt thereof.

12. A method for the treatment of cancer in a subject in need thereof, which comprises administering to the subject an effective amount of the compound of claim 1, or a pharmaceutically acceptable salt thereof.

13. A method for contraception in a male subject, which comprises administering to the male subject an effective amount of the compound of claim 1, or a pharmaceutically acceptable salt thereof.

14. The pharmaceutical composition of claim 10, further comprising one or more additional therapeutic agents selected from anti-cancer agents.

15. The method of claim 12, further comprising administering to the subject one or more additional therapeutic agents selected from anti-cancer agents, wherein the amounts administered of the compound of claim 1 or pharmaceutically acceptable salt thereof, and the one or more additional therapeutic agents, are together effective to treat cancer.
